(19) United States
(12) Patent Application Publication
(10) Pub. No.: US 2022/0387567 A1
(43) Pub. Date: Dec. 8, 2022

(54) COMPOSITIONS AND METHODS FOR TREATING DISEASES AND DISORDERS ASSOCIATED WITH ABERRANT REGULATION OF PROTEINS

(71) Applicants: University of Virginia Patent Foundation, Charlottesville, VA (US); The University of Birmingham, Birmingham (GB); The General Hospital Corporation, Boston, MA (US)

(72) Inventors: Donald F. Hunt, Charlottesville (US); Jeffrey Shabanowitz, Charlottesville (US); Keira Mahoney, New Haven, CT (US); Jennifer G. Abelin, Boston, MA (US); Mohammad Ovais Azizzanjani, Palo Alto, CA (US); Paisley Trantham Myers, Irmo (US); Stacy Alyse Malaker, New Haven, CT (US); Andrew Norris, Oilville (US); Jennifer Hitchcock, Midlothian, VA (US); Xi Peng, Charlottesville (US); Negin Ghafourian, Charlottesville (US); Mark Cobbold, Winchester (US); Sarah Penny, Birmingham (GB); Nico Buettner, Birmingham (GB); James M. Heather, Boston, MA (US)

(73) Assignees: University of Virginia Patent Foundation, Charlottesville, VA (US); The University of Birmingham, Birmingham (GB); The General Hospital Corporation, Boston, MA (US)

(21) Appl. No.: 17/441,662

(22) PCT Filed: Mar. 23, 2020
(86) PCT No.: PCT/US2020/024348

§ 371 Date: Sep. 21, 2021

Related U.S. Application Data
(60) Provisional application No. 62/876,700, filed on Jul. 21, 2019.
(60) Provisional application No. 62/821,468, filed on Mar. 21, 2019.

(51) Int. Cl. A61K 39/00

(52) U.S. Cl. CPC A61K 39/0011; A61K 2039/57; A61K 2039/585

(57) ABSTRACT

Compositions that include anti-cancer, anti-tumor, and anti-microbial infection peptides are provided. In some embodiments, the compositions include 1-10 or more synthetic peptides that are between 8 and 50 amino acids long and include an amino acid sequence as disclosed herein. Also provided are in vitro populations of dendritic cells that include the compositions, in vitro populations of T cells capable of being activated uponbeing brought into contact with the populations of dendritic cells, antibodies and antibody-like molecules that specifically bind to complexes of an MHC class I molecule and the peptides, methods for using the disclosed compositions for treating and/or preventing cancer and/or microbial infections, methods for making cancer vaccines and anti-microbial vaccine, methods for screening peptides for inclusion in immunotherapy compositions, methods for determining a prognosis of a patient with a cancer and/or a microbial infection, kits that include the disclosed peptides, and methods for treating and/or preventing diseases, disorders, and/or conditions associated with hyperphosphorylation of MHC I peptides and/or MHC II peptides, inadequate PP2A activity, and/or undesirable CIP2A activity.

### CROSS REFERENCE TO RELATED APPLICATION

The presently disclosed subject matter claims the benefit of U.S. Provisional Patent Application Ser. No. 62/821,468, filed Mar. 21, 2019, the disclosure of which incorporated herein by reference in its entirety.

### GOVERNMENT INTEREST

This invention was made with government support under Grant No. A1033993 awarded by The National Institutes of Health. The government has certain rights in the invention.

### BACKGROUND

Cells in the human body communicate their health status to the immune system by degrading cellular proteins and presenting fragments of each on the cell surface. The major pathway involves the proteosome, a multi-enzyme particle, not unlike a garbage disposal, that converts the linear protein chain into a mixture, dominated by 9-12 residue peptides. These are then transported into the endoplasmic reticulum via transport associated proteins (TAP). There, one or more chaperone proteins load them onto class I MHC molecules, 47 kiloDalton (kDa) glycoproteins coded by genes in the major histocompatibility complex. A third protein, beta-microglobulin (12 kDa), stabilizes the resulting complex and the trimer is then transported to the cell surface. Appropriately educated, cytotoxic T-lymphocytes (CTL; CD8⁺ T-cells) bind to the class I MHC molecules on the cell surface, sample the peptides being presented and lyse those cells that express new peptides, as a result of viral, bacterial or parasitic infection, tissue transplantation or cellular transformation. Evidence that the immune system plays an active role in the surveillance of tumors includes observations that (a) immunosuppressed transplant recipients display higher incidences of non-viral cancers than appropriate control populations; (b) cancer patients can exhibit spontaneous adaptive and innate immune responses to their tumor; (c) the presence of tumor infiltrating lymphocytes can be a good indicator of survival; and (d) many healthy blood donors have central memory T-cells that respond to and kill cells that present the tumor specific class I and class II phosphopeptide antigens.

Identification of cellular antigens is an important goal because these peptides become potential candidates for vaccines and other cancer treatments such as adoptive-cell therapy (ACT). Unfortunately, sequence analysis of antigenic peptides is a daunting task. Each cell expresses several hundred thousand copies of up to six different class I MHC molecules. Three MHC molecules are inherited from the mother and three from the father. More than a hundred different class I MEW molecules exist in the population at large, but more than eighty percent of the population has one of five common alleles. These are termed HLA-A*0201, HLA-A*0101, HLA-A*0301, HLA-B*0702, and HLA-B*4402. Cells synthesize more than ten thousand different proteins each day and it is expected that one or more fragments from most of these will appear on the cell surface in association with an MEW molecule. Using mass spectrometry, the number of different peptides presented by a given type of class I MEW molecule has been estimated to be between 6,000 and 10,000. Since each cell can present up to 6 different class I MHC molecules, 36,000 to 60,000 different peptides can be displayed on the cell surface at any one time.

CTLs lyse infected or diseased cells that display as few as 5-50 copies of a particular peptide antigen. On 10⁸ cells (100 ml of cell culture), this copy number corresponds to 1-10 fmols of an individual peptide. Diseased cells continue to display the usual number of self peptides along with a small number of additional peptide antigens characteristic of the disease state. The analytical challenge is to be able to identify these antigens in a mixture containing as many as 10,000 self peptides and then sequence them at the low attomole-low femtomole level.

At present, there are several very attractive approaches for immunotherapy of cancer. In 2011, a lentiviral vector that expressed a chimeric construct that contained an antibody receptor for the B-cell antigen CD19 coupled to the CD137 (a costimulatory receptor in T-cells) and CD3-zeta (a signal-transduction component of the T-cell antigen receptor) signaling domains was described. When this vector is transfected into CD8⁺ T cells, they recognize and kill cells that express the surface protein antigen CD19. Remarkably, late stage chronic lymphocytic leukemia (CLL) patients were cured of their disease in a matter of weeks by this approach. Unfortunately, the treatment also wiped out normal B-cells and left the patients with compromised immune systems.

To date, the most effective treatment for metastatic melanoma has been adoptive cell therapy (ACT). In this approach, tumor infiltrating lymphocytes (TIL) are isolated from resected tumors and expanded to large numbers (1×10¹⁰ cells) in vitro. After the patient's immune system is ablated by a combination of chemotherapy and total body irradiation, the TIL, plus the cytokine interleukin-2 (IL-2), are re-infused and allowed to search out the tumor in the absence of CD4⁺ Treg cells. Objective (tumor shrinkage) and complete responses for this therapy in a recent clinical trial of 25 late-stage patients with metastatic melanoma were 72% and 16%, respectively. Efforts to improve this technology are in progress and involve transfecting patient CD8⁺ T-cells (prior to expansion) with high affinity receptors for specific melanoma associated Class I MHC peptides (MART 1, etc.).

Striking data on the treatment of cancer with immune-mobilizing monoclonal T cell receptors (ImmTACs) has recently been published. Here, the approach is to use phage display technology to engineer a specific CD8⁺ T cell receptor (extracellular portion) so that it has antibody-like affinity (i.e., picomolar instead of micromolar affinity) and then couple it to a humanized CD3-specific scFv sequence that will trigger killing by any polyclonal T-cell in the vicinity of bound receptor. Outstanding results have been obtained on melanoma in vitro with a receptor that recognizes the class I peptide YLEPGPVTA (SEQ ID NO: 3222) from the protein gp100 on HLA-A*0201. Use of the ImmTAC for YLEPGPVTA (SEQ ID NO: 3222) is presently being evaluated in a phase II clinical trial on melanoma patients.

Another approach to immunotherapy of cancer is based on the finding that human tumors harbor a remarkable number of somatic mutations. Class I MHC peptides that contain these mutations (neoantigens) should be recognized as non-self and trigger T-cells to kill the cells that present them. To find these neoantigens, individual patient tumors are subjected to whole exome sequencing and a combination of prediction algorithms, analysis of eluted MHC peptides by mass spectrometry, and large scale peptide synthesis is employed to define which mutated peptides are presented by specific HLA alleles. The result is a personalized vaccine for each cancer patient. Unfortunately, to date very few of these mutated antigens are shared by multiple tumors.

Also of note are recent cancer therapies based on antibodies that recognize cell surface proteins involved in down regulating the immune response to tumor antigens, thus preventing collateral tissue damage and autoimmune disease. Ipilimumab targets cytotoxic T-lymphocyte associated antigen-4 (CTLA4) and up-regulates the amplitude of the early stages of T cell activation. It received FDA approval for treatment of melanoma in 2010. Another immune-checkpoint receptor programmed cell death protein 1 (PD1) limits the activity of T-cells in the peripheral tissues and is also highly expressed on Treg cells. An antibody directed to this receptor blocks immune suppression. Objective responses were observed in a recent clinical trial with this antibody on patients with melanoma, non-small cell lung cancer, and renal cell cancer. A recent treatment with anti PD1 antibody cured former U.S. President Carter of metastatic melanoma.

There is a long felt need in the art for compositions and methods useful for treating and preventing diseases and disorders associated with aberrant expression and regulation of class I MHC peptides, particularly phosphopeptides, as well as aberrant regulation and post-translational modification of other proteins. The presently disclosed subject matter satisfies these needs.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter discloses in part that loss or dysregulation of PP2A expression or activity is associated with diseases and disorders due to hyperphosphorylation of peptides and that other disease and disorders are associated with aberrant methylation on Arg and Lys or O-GlcNAcylation on Ser and Thr. In some embodiments, the presently disclosed subject matter provides compositions and methods for determining whether a disease, disorder, and/or condition is associated with hyperphosphorylation of MHC I peptides or other peptides or proteins or aberrant methylation on Arg and Lys or O-GlcNAcylation on Ser and Thr. In some embodiments, the presently disclosed subject matter provides targets for treatment and methods for identifying those targets.

The presently disclosed subject matter provides, inter alia, Class I MHC phosphopeptide neoantigens and compositions and methods for identifying such antigens, sequencing the antigens, and treating subjects with aberrant regulation of the antigens. In some embodiments, they are post-translationally modified. In some embodiments, Class II peptides are identified and used.

The presently disclosed subject matter provides compositions and method useful for preventing and treating diseases, disorders, and/or conditions, in some embodiments cancer and in some embodiments and microbial infections, which are associated with aberrant expression, aberrant regulation, and aberrant post-translational modification of peptides or proteins, including class I MHC peptides. In some embodiments, there are two or more problems or defects in aberrant expression, regulation, or post-translational modification in a subject. In some embodiments, the peptides are phosphopeptides. In some embodiments, aberrant expression of a class I MHC peptide is in a cancer cell or a microbial infected cell, including a bacterial infected cell or a virus infected cell. In some embodiments, the subject has been infected with a bacteria or a virus, or more than one bacteria, virus, or a combination thereof.

In some embodiments, the virus is selected from the group consisting of HIV, HPV, HCV, HBV, EBV, MCPyV, and coronavirus, which in some embodiments can be SARS-CoV and/or SARS-CoV-2 and/or MERS-CoV.

In some embodiments, the bacteria is selected from the group consisting of H. pylori, Fusobacterium nucleatum, and other bacteria of the gastrointestinal microbiome. In some embodiments, the aberrant regulation is of a signaling pathway.

In some embodiments, post-translational modification includes, but is not limited to, phosphorylation, methylation on Arg and Lys, and O-GlcNAcylation on Ser and Thr.

In some embodiments, viruses or bacteria cause infected cells to present multiple class I MHC phosphopeptide neoantigens.

In some embodiments, the presently disclosed subject matter provides compositions and methods for detecting and for preventing and treating diseases and disorders where PP2A has been inactivated or has decreased effects or activity. In some embodiments, there is aberrant regulation of PP2A. In some embodiments, the aberrant regulation is inhibition of PP2A activity, expression, or levels. Compositions and methods of the presently disclosed subject matter are useful for reversing or inhibiting diseases and disorders due to hyperphosphorylation of peptides and other disease and disorders that are associated with aberrant methylation on Arg and Lys or O-GlcNAcylation on Ser and Thr.

Many phosphopeptides: (a) are uniquely expressed on tumors and not on normal cells, (b) are found on multiple types of cancer, (c) are recognized by central memory T-cells in PBMC from healthy blood donors, and (d) trigger killing by cytotoxic T-cells.

The presently disclosed subject matter provides compositions and methods for the treatment of disease that targets Class I and/or Class II MHC phosphopeptides that are in some embodiments uniquely presented on the cell surface because one or more phosphatases in the diseased cell are inhibited.

The diseases and disorders that can be prevented or treated by the compositions and methods of the presently disclosed subject matter include, but are not limited to, cancer, Alzheimer's disease, and infections, including, but not limited to, bacterial infections and viral infections. Cancers that can be prevented or treated include, but are not limited to, leukemia (several types, including, for example, AML, ALL, and CLL), melanoma, breast, ovarian, colorectal, esophageal, and hepatocellular cancers.

Many tumors that exhibit aberrant expression of class I MHC phosphopeptides or class I MHC peptides are known in the art. See, for example, PCT International Patent Application Publication Nos. WO 2014/036562, WO 2014/039675, WO 2014/093855, WO 2015/034519, and WO 2015/120036; U.S. Patent Application Publication Nos. 2008/0153112, 2010/0297158, 2013/0259883, 2015/0328297, 2016/0000893, 2017/0029484, 2018/0066017, 2019/0015494, and 2019/0374627, and U.S. Pat. Nos. 8,119,984; 8,211,436, 8,692,187; 9,171,707; 9,279,011; 9,561,266; 10,281,473; each of which is incorporated by reference herein in its entirety, for useful peptides and methods. Other post-translational modifications are also encompassed by the presently disclosed subject matter.

In some embodiments, the presently disclosed subject matter provides compositions and methods for preventing and treating diseases and disorders where PP2A has been inactivated or has decreased effects or activity. In some embodiments, the loss or decreased levels of PP2A or PP2A activity results from loss of decreased levels of RB-1 effects or activity. In some embodiments, the loss or decreased levels of PP2A or PP2A activity results from induction or enhanced levels of CIP2A effects or activity. In some embodiments, the loss or decreased levels of PP2A or PP2A activity results in an increase in phosphorylation of class I MHC peptides and an increase in cell surface expression of the phosphopeptides.

In some embodiments, the loss or decreased levels of PP2A or PP2A activity results in neurodegeneration. In some embodiments, the loss or decreased levels of PP2A or PP2A activity results in hyperphosphorylation of a peptide such as Tau and is associated with Alzheimer's disease. In some embodiments, the presently disclosed subject matter provides compositions and methods to inhibit hyperphosphorylation of Tau or to reverse hyperphosphorylation of Tau that has been hyperphosphorylated.

Based on the discoveries presented herein, several types of treatments can be used where there is a disease, disorder, and/or condition associated with the loss or decreased levels of PP2A or PP2A activity. These include first identifying hyperphosphorylated or abnormally post-translationally modified peptides in a subject. Then, the peptides can be purified and used as immunogens and/or once identified can be synthesized and used as immunogens, and/or cells and/or tissues can be isolated and the peptides at least partially purified and used as immunogens. The presently disclosed subject matter further encompasses methods to restore PP2A levels or activity, to dephosphorylate any hyperphosphorylated peptides that resulted from inhibition of PP2A, etc.

In some embodiments, the treatment of the presently disclosed subject matter is an immunotherapy.

In some embodiments, the presently disclosed subject matter provides compositions and methods useful as a vaccine or as an immunogen for cancer or other diseases, disorders, and/or conditions.

In some embodiments, the presently disclosed subject matter provides compositions and methods useful as a therapeutic for treating cancer or as a vaccine for preventing cancer in a subject in need thereof.

In some embodiments, the presently disclosed subject matter provides compositions and methods useful as a therapeutic for treating a microbial infection or as a vaccine for preventing a microbial infection in a subject in need thereof.

In some embodiments, identified hyperphosphorylated peptides can be isolated or synthesized and administered to a subject as a therapeutic for treating a disease, disorder, and/or condition or as a vaccine for the disease or disorder. In some embodiments, peptides or proteins with other aberrant post-translations modifications associated with a disease, disorder, and/or condition can be isolated or synthesized and administered to a subject as a therapeutic for treating a disease, disorder, and/or condition or as a vaccine for the disease or disorder.

Several in vitro and in vivo assays can be used to demonstrate the effectiveness of the peptides of the presently disclosed subject matter and are disclosed herein or in the references cited herein below.

Various aspects and embodiments of the presently disclosed subject matter are described in further detail herein below.

### BRIEF DESCRIPTION OF THE FIGURES

FIG. 1 is a schematic of an exemplary method for isolating and analyzing modified peptides as per the presently disclosed subject matter.

FIG. 2 is a schematic of an exemplary methods for determining the sequencing of as well as the phosphosite of a phosphopeptide as per the presently disclosed subject matter.

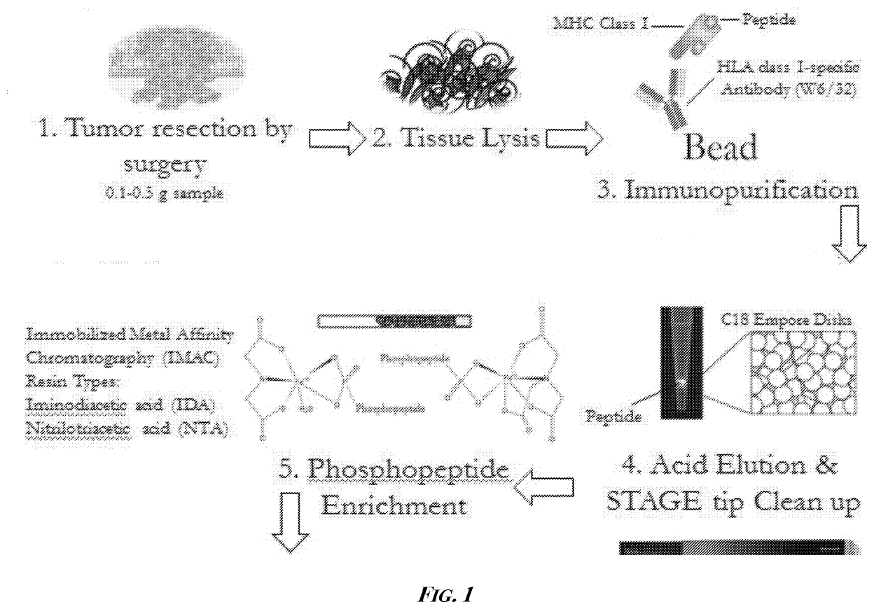

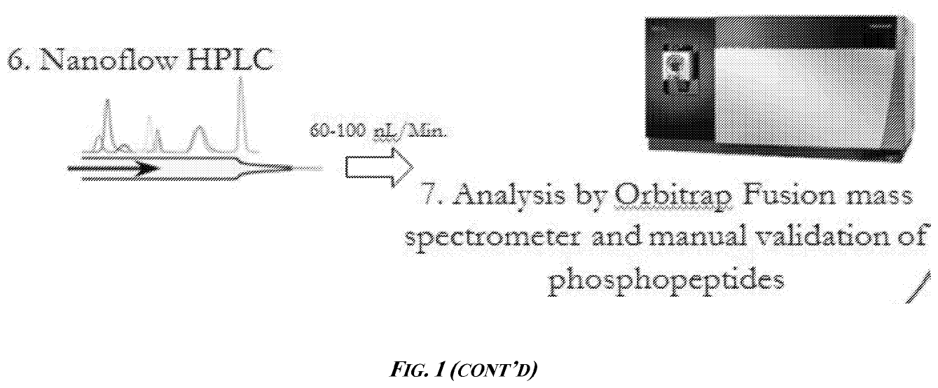

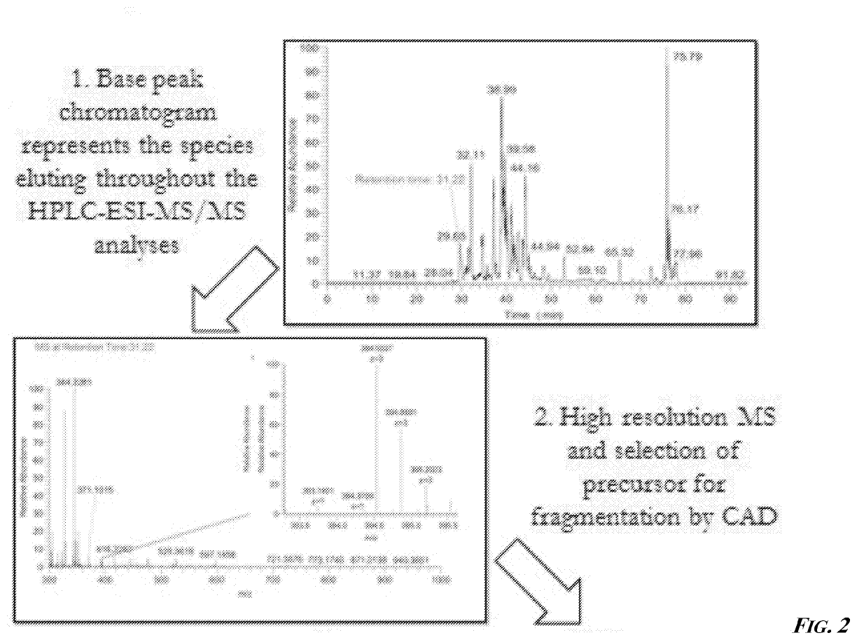

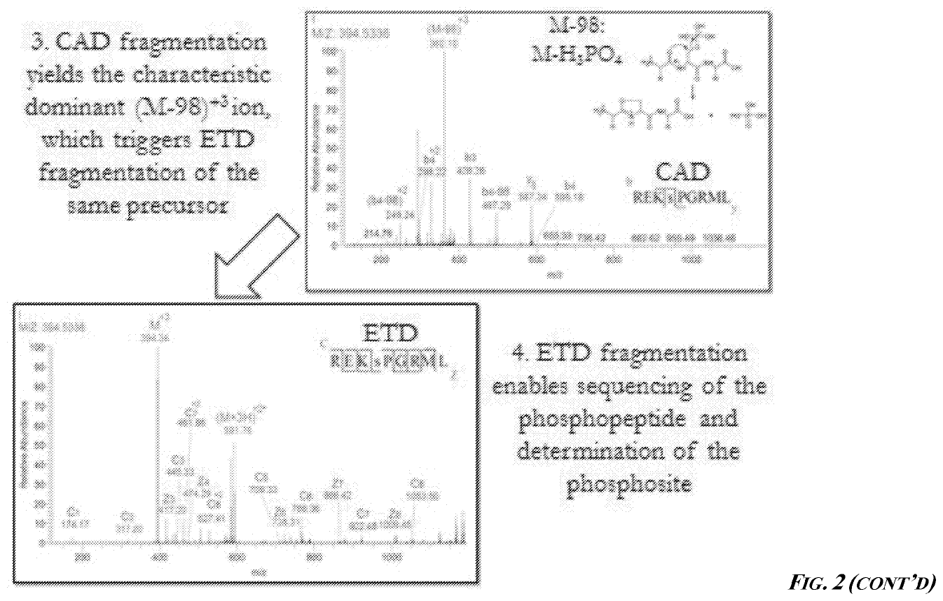

### DETAILED DESCRIPTION

Phosphopeptide antigens are of considerable therapeutic interest because to dysregulation of protein kinase activity, normally tightly controlled, plays a prominent role in the hallmark traits of cancer. These include sustained proliferative signaling, evasion of growth suppressors, resistance to apoptotic signals, unlimited replicative potential, induction of angiogenesis, activation of invasion and metastasis, reprogramming of energy metabolism, and eventual evasion of the immune system. These considerations suggest that alterations in protein phosphorylation (also including O-GlcNAcylation and/or methylation) are likely to occur during malignancy. Without wishing to be bound by any particular theory it is hypothesized herein that Class I and Class II phosphopeptides produced by dysregulated signaling pathways in the tumor should not be found in a normal tissue such as the thymus or lymph nodes. As a consequence, tolerance (deletion of high avidity T-cells) to these antigens is highly unlikely. If the kinase or target protein is required for the transformation process, neoangiogenesis, metastasis, or another critical tumor function, tumor escape by mutation or gene deletion without compromising tumor survival is also unlikely.

### I. Definitions

Headings are included herein for reference and to aid in locating certain sections. These headings are not intended to limit the scope of the concepts described therein under, and these concepts may have applicability in other sections throughout the entire specification. While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, In some embodiments, the term “about” is used herein to modify a numerical value above and below the stated value by a variance of in some embodiments ±10% and in some embodiments ±20%. Therefore, about 50% means in some embodiments in the range of 45%-55% and in some embodiments in the range of 40-60%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease, disorder, and/or condition which may not be responsive to the primary treatment for the injury, disease, disorder, and/or condition being treated.

As used herein, the term “adjuvant” refers to a substance that elicits an enhanced immune response when used in combination with a specific antigen.

As use herein, the terms “administration of” and or “administering” a compound should be understood to mean providing a compound of the presently disclosed subject matter or a prodrug of a compound of the presently disclosed subject matter to a subject in need of treatment.

As used herein, an “agonist” is a composition of matter which, when administered to a mammal such as a human, enhances or extends a biological activity attributable to the level or presence of a target compound or molecule of interest in the mammal.

A disease, disorder, and/or condition is “alleviated” if the severity of a symptom of the disease, condition, or disorder, or the frequency with which such a symptom is experienced by a subject, or both, are reduced.

As used herein, “amino acids” are represented by the full name thereof, by the three letter code corresponding thereto, or by the one-letter code corresponding thereto, as known to those of ordinary skill. The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy-or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter. The term “amino acid” is also interchangeably with “amino acid residue”, and may refer to a free amino acid and to an amino acid residue of a peptide. It will be apparent from the context in which the term is used whether it refers to a free amino acid or a residue of a peptide.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:
Small aliphatic, nonpolar or slightly polar residues: Ala, Ser, Thr, Pro, Gly;
Polar, negatively charged residues and their amides: Asp, Asn, Glu, Gln, cysteic acid and homocysteic acid;
Polar, positively charged residues: His, Arg, Lys; Ornithine (Orn)
Large, aliphatic, nonpolar residues: Met, Leu, Ile, Val, Cys, Norleucine (Nle), homocysteine
Large, aromatic residues: Phe, Tyr, Trp, acetyl phenylalanine

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the mammal.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample”, as used herein, refers to samples obtained from a subject, including, but not limited to, skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate may, for example, be redeposited as bone mineral, or by being otherwise re-utilized within the body, or excreted. “Strongly bioresorbable”, as the term is used herein, means that at least 80% of the total mass of material implanted is resorbed within one year.

The term “cancer”, as used herein, is defined as proliferation of cells whose unique trait—loss of normal growth controls—results in unregulated growth, lack of differentiation, local tissue invasion, and metastasis. Examples include but are not limited to, leukemia, melanoma, breast cancer, prostate cancer, ovarian cancer, uterine cancer, cervical cancer, skin cancer, pancreatic cancer, colorectal cancer, renal cancer, and lung cancer.

As used herein, the term “carrier molecule” refers to any molecule that is chemically conjugated to the antigen of interest that enables an immune response resulting in antibodies specific to the native antigen.

The terms “cell culture” and “culture,” as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium”, “culture medium” (plural “media” in each case) and “medium formulation” refer to a nutritive solution for cultivating cells and may be used interchangeably.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease, disorder, and/or condition for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease, disorder, and/or condition in the animal in which the tissue is located (or from which the tissue was obtained).

A tissue “normally comprises” a cell if one or more of the cell are present in the tissue in an animal not afflicted with a disease or disorder.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990 modified as in Karlin & Altschul, 1993. This algorithm is incorporated into the NBLAST and XBLAST programs of Altschul et al. (1990), and can be accessed, for example at the National Center for Biotechnology Information (NCBI) world wide web site. BLAST nucleotide searches can be performed with the NBLAST program (designated “BLASTN” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “BLASTN” at the NCBI web site) or the NCBI “BLASTP” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit”, as used herein when referring to a function, refers to the ability of a compound of the presently disclosed subject matter to reduce or impede a described function. Preferably, inhibition is by at least 10%, more preferably by at least 25%, even more preferably by at least 50%, and most preferably, the function is inhibited by at least 75%. When the term “inhibit” is used more generally, such as “inhibit Factor I”, it refers to inhibiting expression, levels, and activity of Factor I.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound the presently disclosed subject matter or be shipped together with a container which contains the identified compound. Alternatively, the instructional material may be shipped separately from the container with the intention that the instructional material and the compound be used cooperatively by the recipient.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides. In some embodiments, a peptide of the presently disclosed subject matter includes at least 6 and as many as 50, 75, or 100 amino acids.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human).Those of ordinary skill in the art will understand and appreciate the techniques appropriate for determining whether an active ingredient has a desired efficacious outcome based upon the needs of the artisan.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application.

As used herein, the term “physiologically acceptable” ester or salt means an ester or salt form of the active ingredient which is compatible with any other ingredients of the pharmaceutical composition, which is not deleterious to the subject to which the composition is to be administered.

As used herein, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refer to non-naturally occurring peptides or polypeptides. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups. With respect to a terminal carboxy group, “protecting group” refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides, which in some embodiments are polypeptides of greater than 100 amino acids. Conventional notation is used herein to portray polypeptide sequences: the left-hand end of a polypeptide sequence is the amino-terminus (N-terminus); the right-hand end of a polypeptide sequence is the carboxy- or carboxyl-terminus (C-terminus).

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

A “recombinant polypeptide” is one which is produced upon expression of a recombinant polynucleotide.

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, more preferably by at least 25%, and even more preferably by at least 50%. The term “stimulator” as used herein, refers to any composition, compound or agent, the application of which results in the stimulation of a process or function of interest.

A “subject” of analysis, diagnosis, or treatment is an animal. Such animals include in some embodiments mammals, which in some embodiments can be a human.

As used herein, a “subject in need thereof” is a patient, animal, mammal, or human, who will benefit from the compositions and methods of the presently disclosed subject matter.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine,” as used herein, is meant a composition which when inoculated into a subject has the effect of stimulating an immune response in the subject, which serves to fully or partially protect the subject against a disease, disorder, or condition or at least one of its symptoms. In some embodiments, the disease, disorder, or condition is cancer. In some embodiments, the disease, disorder, or condition is a microbial infect, which in some embodiments can be a bacterial infection and in some embodiments can be a viral infection. The term vaccine encompasses prophylactic as well as therapeutic vaccines. A combination vaccine is one which combines two or more vaccines, or two or more compounds or agents.

### II. Peptides and Modified Peptides

The presently disclosed subject matter relates in some embodiments to immunogenic therapeutic peptides for use in immunotherapy and diagnostic methods of using the peptides, as well as methods of selecting the same to make compositions for immunotherapy, e.g., in vaccines and/or in compositions useful in adaptive cell transfer. In some embodiments, the peptides of the presently disclosed subject matter are post-translationally modified by being provided with a phosphate group, (i.e., “phosphopeptides”). In some embodiments, the peptides of the presently disclosed subject matter are summarized in Table 6 and/or Table 7 herein below.

The peptides of the presently disclosed subject matter are in some embodiments not the entire proteins from which they are derived. They are in some embodiments from 6 to 50 contiguous amino acid residues of the native human protein. They can in some embodiments contain exactly, about, or at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. The peptides of the presently disclosed subject matter can also in some embodiments have a length that falls in the ranges of 6-10, 9-12, 10-13, 11-14, 12-15, 15-20, 20-25, 25-30, 30-35, 35-40, and 45-50 amino acids. Exactly, about, or at least 1, 2, 3, 4, 5, 6, 7, 8, 9, 10, 11, 12, 13, 14, 15, 16, 17, or more of the amino acid residues within the recited sequence of a peptide can phosphorylated. Peptides can be modified and analogs (using for example, beta-amino acids, L-amino acids, N-methylated amino acids, amidated amino acids, non-natural amino acids, retro inverse peptides, peptoids, PNA, halogenated amino acids) can be synthesized that retain their ability to stimulate a particular immune response, but which also gain one or more beneficial features, such as those described below. Thus, particular peptides can, for example, have use for treating and vaccinating against multiple cancer types.

In some embodiments, substitutions can be made in the peptides at residues known to interact with the MHC molecule. Such substitutions can in some embodiments have the effect of increasing the binding affinity of the peptides for the MHC molecule and can also increase the half-life of the peptide-MHC complex, the consequence of which is that the analog is in some embodiments a more potent stimulator of an immune response than is the original peptide.

Additionally, the substitutions can in some embodiments have no effect on the immunogenicity of the peptide per se, but rather can prolong its biological half-life or prevent it from undergoing spontaneous alterations which might otherwise negatively impact on the immunogenicity of the peptide.

The peptides disclosed herein can in some embodiments have differing levels of immunogenicity, MHC binding and ability to elicit CTL responses against cells displaying a native peptide, e.g., on the surface of a tumor cell.

The amino acid sequences of the peptides can in some embodiments be modified such that immunogenicity and/or binding is enhanced. In some embodiments, the modified peptide binds an MHC class I molecule about or at least 10%, 20%, 30%, 40%, 50%, 60%, 70%, 80%, 90%, 95%, 100%, 110%, 125%, 150%, 175%, 200%, 225%, 250%, 275%, 300%, 350%, 375%, 400%, 450%, 500%, 600%, 700%, 800%, 1000%, or more tightly than its native (unmodified) counterpart.

However, given the exquisite sensitivity of the T-cell receptor, it cannot be foreseen whether such enhanced binding and/or immunogenicity will render a modified peptide still capable of inducing an activated CTL that will cross react with the native peptide being displayed on the surface of a tumor. Indeed, it is disclosed herein that the binding affinity of a peptide does not predict its functional ability to elicit a T cell response.

Peptides of the presently disclosed subject matter can in some embodiments be mixed together to form a cocktail. The peptides can in some embodiments be in an admixture, or they can in some embodiments be linked together in a concatemer as a single molecule. Linkers between individual peptides can in some embodiments be used; these can, for example, in some embodiments be formed by any 10 to 20 amino acid residues. The linkers can in some embodiments be random sequences, or they can in some embodiments be optimized for degradation by dendritic cells.

In certain specified positions, a native amino acid residue in a native human protein can in some embodiments be altered to enhance the binding to the MHC class I molecule. These can occur in “anchor” positions of the peptides, often in positions 1, 2, 3, 9, or 10. Valine (V), alanine (A), lysine (K), leucine (L), isoleucine (I), tyrosine (Y), arginine (R), phenylalanine (F), proline (P), glutamic acid (E), glutamine (Q), threonine (T), serine (S), aspartic acid (D), tryptophan (W), and methionine (M) can also be used in some embodiments as improved anchoring residues. Anchor residues for different HLA molecules are listed below. Anchor residues for exemplary HLA molecules are listed in Table 1.

TABLE 1
Anchor Residues for Different HLA Molecules

| | | | | | Residue |
|---|---|---|---|---|---|
| | Residue | Residue | Residue | Residue | 9 or Last |
| | 1 | 2 | 3 | 7 | Residue |
| HLA A*0101 | | T, S | D, E | | Y |
| HLA A*0201 | | L, M | | | V |
| HLA A*0301 | | L, M | | | K |
| HLA A*24 | | Y, W, M | | | L, F, W |
| HLA B*0702 | | P | | | L, M, V, F |
| HLA B*1508 | | P, A | | | Y |
| HLA B*2705 | R | R | | | L, F, K, R, M |
| HLA B*4402 | | E | | | F, Y, W |
| HLA C*0501 | Y | P, A | D | | F, I, L, M, V |
| HLA C*0602 | F, Y | R, Y | A, F, Y | K, Q, R | I, L, M, V |

In some embodiments, the immunogenicity of a peptide is measured using transgenic mice expressing human MHC class I genes. For example, “ADD Tg mice” express an interspecies hybrid class I MHC gene, AAD, which contains the alpha-1 and alpha-2 domains of the human HLA-A2.1 gene and the alpha-3 transmembrane and cytoplasmic domains of the mouse H-2Dd gene, under the direction of the human HLA-A2.1 promoter. Immunodetection of the HLA-A2.1 recombinant transgene established that expression was at equivalent levels to endogenous mouse class I molecules. The mouse alpha-3 domain expression enhances the immune response in this system. Compared to unmodified HLA-A2.1, the chimeric HLA-A2.1/H2-Dd MHC Class I molecule mediates efficient positive selection of mouse T cells to provide a more complete T cell repertoire capable of recognizing peptides presented by HLA-A2.1 Class I molecules. The peptide epitopes presented and recognized by mouse T cells in the context of the HLA-A2.1/H2-Dd class I molecule are the same as those presented in HLA-A2.1⁺ humans. This transgenic strain facilitates the modeling of human T cell immune responses to HLA-A2 presented antigens, and identification of those antigens. This transgenic strain is a preclinical model for design and testing of vaccines for infectious diseases or cancer therapy involving optimal stimulation of CD8⁺ cytolytic T cells.

In some embodiments, the immunogenicity of a modified peptide is determined by the degree of Interferon gamma and/or TNF-α production of T-cells from ADD Tg mice immunized with the peptide, e.g., by immunization with peptide pulsed bone marrow derived dendritic cells.

In some embodiments, the modified peptides are about or at least 10%, 20%, 30%, 40%, 50%, 60%, 70%, 80%, 90%, 95%, 100%, 110%, 125%, 150%, 175%, 200%, 225%, 250%, 275%, 300%, 350%, 375%, 400%, 450%, 500%, 600%, 700%, 800%, 1000%, 1500%, 2000%, 2500%, 3000%, 4000%, 5000%, or more immunogenic, e.g., in terms of numbers of Interferon gamma and/or TNF-alpha positive (i.e., “activated”) T-cells relative to numbers elicited by native peptides in ADD Tg mice immunized with peptides pulsed bone marrow derived dendritic cells. In some embodiments, the modified peptides are able to elicit CD8⁺ T cells which are cross-reactive with the modified and the native peptide in general and when such modified and native peptides are complexed with MHC class I molecules in particular. In some embodiments, the CD8⁺ T cells which are cross-reactive with the modified and the native peptides are able to reduce tumor size by about or at least 10%, 20%, 30%, 40%, 50%, 60%, 70%, 80%, 90%, 95%, 97%, or 99% in a NOD/SCID/IL-2Rγc^{-/-} knock out mouse (which has been provided transgenic T cells specific form an immune competent donor) relative to IL-2 treatment without such cross-reactive CD8⁺ T cells.

The term “capable of inducing a peptide-specific memory T cell response in a patient” as used herein relates to eliciting a response from memory T cells (also referred to as “antigen-experienced T cell”) which are a subset of infection- and cancer-fighting T cells that have previously encountered and responded to their cognate antigen. Such T cells can recognize foreign invaders, such as bacteria or viruses, as well as cancer cells. Memory T cells have become “experienced” by having encountered antigen during a prior infection, encounter with cancer, or previous vaccination. At a second encounter with the cognate antigen, e.g., by way of an initial inoculation with a peptide of the presently disclosed subject matter, memory T cells can reproduce to mount a faster and stronger immune response than the first time the immune system responded to the invader (e.g., through the body's own consciously unperceived recognition of a peptide being associated with diseased tissue). This behavior can be assayed in T lymphocyte proliferation assays, which can reveal exposure to specific antigens. Memory T cells comprise two subtypes: central memory T cells (T_{CM} cells) and effector memory T cells (T_{EM} cells). Memory cells can be either CD4⁺ or CD8⁺. Memory T cells typically express the cell surface protein CD45RO. Central memory T_{CM} cells generally express L-selectin and CCR7, they secrete IL-2, but not IFNγ or IL-4. Effector memory TEM cells, however, generally do not express L-selectin or CCR7 but produce effector cytokines like IFNγ and IL-4.

A memory T cell response generally results in the proliferation of memory T cell and/or the upregulation or increased secretion of the factors such as CD45RO, L-selectin, CCR7, IL-2, IFNγ, CD45RA, CD27, and/or IL-4. In some embodiments, the peptides of the presently disclosed subject matter are capable of inducing a TCM cell response associated with L-selectin, CCR7, IL-2 (but not IFNγ or IL-4) expression and/secretion (see e.g., Hamann et al., 1997). In some embodiments, a TCM cell response is associated with an at least or about 1%, 5%, 10%, 15%, 20%, 25%, 30%, 35%, 45%, 50%, 55%, 60%, 65%, 70%, 75%, 80%, 90%, 95%, 97%, 98%, 99%, 100%, 125%, 150%, 175%, 200%, 250%, 300%, 400%, 500%, 600%, 700%, 800%, 900%, 1000%, 1500%, 2000%, or more increase in T cell CD45RO/RA, L-selectin, CCR7, or IL-2 expression and/secretion.

In some embodiments, the peptides of the presently disclosed subject matter are capable of inducing a CD8⁺ T_{CM} cell response in a patient the first time that patient is provided the composition including the selected peptides. As such, the peptides of the presently disclosed subject matter can in some embodiments be referred to as “neo-antigens”. Although peptides might be considered “self” for being derived from self-tissue, they generally are only found on the surface of cells with a dysregulated metabolism, e.g., aberrant phosphorylation, they are likely never presented to immature T cells in the thymus. As such, these “self” antigens act are neo-antigens because they are nevertheless capable of eliciting an immune response.

In some embodiments, about or at least 1%, 5%, 10%, 15%, 20%, 25%, 30%, 35%, 45%, 50%, 55%, 60%, 65%, 70%, 75%, 80%, 90%, 95%, 97%, 98%, or 99% of T cells activated by particular peptide in a particular patient sample are T_{CM} cells. In some embodiments, a patient sample is taken exactly, about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days after an initial exposure to a particular peptide and then assayed for peptide specific activated T cells and the proportion of T_{CM} cells thereof. In some embodiments, the compositions of the presently disclosed subject matter are able to elicit a CD8⁺ T_{CM} cell response in at least or about 1%, 5%, 10%, 15%, 20%, 25%, 30%, 35%, 45%, 50%, 55%, 60%, 65%, 70%, 75%, 80%, 90%, 95%, 97%, 98%, 99%, or 100% of patients and/or healthy volunteers. In some embodiments, the compositions of the presently disclosed subject matter are able to elicit a CD8⁺ T_{CM} cell response in a patient about or at least 1%, 5%, 10%, 15%, 20%, 25%, 30%, 35%, 45%, 50%, 55%, 60%, 65%, 70%, 75%, 80%, 90%, 95%, 97%, 98%, 99%, or 100% of patients and/or healthy volunteers specific to all or at least or about 1, 2, 3, 4, 5, 6, 7, 8, 9, or 10 peptides in the composition. In some embodiments, the aforementioned T cell activation tests are done by ELISpot assay.

In some embodiments, the peptides of the presently disclosed subject matter are post-translationally-modified by being provided with a phosphate group (referred to herein as “phosphopeptides”). The term “phosphopeptides” includes MHC class I-specific phosphopeptides. Exemplary MEW class I phosphopeptides of the presently disclosed subject matter that are associated in some embodiments with hepatocellular carcinoma are set forth in Tables 6 and 7. In Tables 6 and 7, phosphoserine, phosphothreonine, and phosphotyrosine residues are indicated by “s”, “t”, and “y”, respectively. It is noted, however, that serine, threonine, and tyrosine residues depicted in uppercase “S”, “T”, and “Y” can also be modified, for example by phosphorylation, and further that in peptides with a plurality of serine/threonine/tyrosine residues, each and every combination and subcombination of serine, threonine, and tyrosine residues can be replaced with phosphoserine, phosphothreonine/ore, and phosphotyrosine residues. A lowercase “c” in a peptide sequence indicates that in some embodiments the cysteine is present in a cysteine-cysteine disulfide bond at the surface of a cell and, in some embodiments, is presented to the immune system as such.

In some embodiments, the phosphopeptides of the presently disclosed subject matter comprise the amino acid sequences of at least one of the MEW class I binding peptides set forth in SEQ ID NOs: 1-3921 and 3975-4000. Moreover, in some embodiments about or at least 1, 2, 3, 4, 5, 6, 7, 8, 9, 10, 11, 12, 13, 14, 15, or more of the serine, homo-serine, threonine, or tyrosine residues within the recited sequence is phosphorylated. The phosphorylation can in some embodiments be with a natural phosphorylation (—CH₂—O—PO₃H) or with an enzyme non-degradable, modified phosphorylation, such as (—CH₂—CF₂—PO₃H or —CH₂—CH₂—PO₃H). Some phosphopeptides can contain more than one of the amino acid sequences set forth in SEQ ID NOs: 1-3921 and 3975-4000, for example, if they are overlapping, adjacent, or nearby within the native protein from which they are derived.

In some embodiments, the peptides comprise a phosphopeptide mimetic. In some embodiments, the phosphopeptide mimetic replaces a phosphoserine, phosphothreonine, or phosphotyrosine residue indicated in Tables 6 and 7. The chemical structure of a phosphopeptide mimetic appropriate for use in the presently disclosed subject matter can in some embodiments closely approximate the natural phosphorylated residue which is mimicked, and also can in some embodiments be chemically stable (e.g., resistant to dephosphorylation by phosphatase enzymes). This can be achieved with a synthetic molecule in which the phosphorous atom is linked to the amino acid residue, not through oxygen, but through carbon. In some embodiments, a CF₂ group links the amino acid to the phosphorous atom. Mimetics of several amino acids which are phosphorylated in nature can be generated by this approach. Mimetics of phosphoserine, phosphothreonine, and phosphotyrosine can be generated by placing a CF₂ linkage from the appropriate carbon to the phosphate moiety. The mimetic molecule L-2-amino-4 (diethylphosphono)-4,4-difluorobutanoic acid (F2Pab) can in some embodiments substitute for phosphoserine (Otaka et al., 1995). L-2-amino-4-phosphono-4,4difluoro-3-methylbutanoic acid (F2Pmb) can in some embodiments substitute for phosphothreonine. L-2-amino-4-phosphono (difluoromethyl) phenylalanine (F2Pmp) can in some embodiments substitute for phosphotyrosine (Smyth et al., 1992; Akamatsu et al., 1997). Alternatively, the oxygen bridge of the natural amino acid can in some embodiments be replaced with a methylene group. In some embodiments, serine and threonine residues are substituted with homo-serine and homo-threonine residues, respectively. A phosphomimetic can in some embodiments also include vanadate, pyrophosphate or fluorophosphates.

### III. Immunosuitablity

In some embodiments, the peptides of the presently disclosed subject matter are combined into compositions which can be used in vaccine compositions for eliciting anti-tumor immune responses or in adoptive T-cell therapy of cancer patients and/or patients with microbial infections. Tables 3-7 provide peptides presented on the surface of cancer cells.

The presently disclosed subject matter provides in some embodiments peptides which are immunologically suitable for each of the foregoing HLA alleles and, in particular, HLA-A*0201 molecule, an HLA A*0101 molecule, an HLA A*0301 molecule, an HLA B*4402 molecule, an HLA B*0702 molecule, an HLA B*2705 molecule, an HLA *A1101 molecule, an HLA *A2301 molecule, an HLA *A2402 molecule, an HLA *B0801 molecule, an HLA *B1401 molecule, an HLA *B1402 molecule, an HLA *B1501 molecule, an HLA *B1503 molecule, an HLA *B1510 molecule, an HLA *B1511 molecule, an HLA *B1518 molecule, an HLA *B4001 molecule, an HLA *B4901 molecule, an HLA *C0303 molecule, an HLA *C0304 molecule, an HLA *C0501 molecule, an HLA *0602 molecule, an HLA *0701 molecule, an HLA *0702 molecule, and an HLA *0704 molecule. “Immunologically suitable” means that a peptide will bind at least one allele of an MEW class I molecule and/or an MEW class II molecule in a given patient. Compositions of the presently disclosed subject matter are in some embodiments immunologically suitable for a patient when at least one peptide of the composition will bind at least one allele of an MEW class I molecule and/or an MHC class II moleculein a given patient. Compositions of multiple peptides presented by each of the most prevalent alleles used in a cocktail, ensures coverage of the human population and to minimize the possibility that the tumor will be able to escape immune surveillance by down-regulating expression of any one class I and/or class II peptide.

The compositions of the presently disclosed subject matter can in some embodiments have at least one peptide specific for HLA-A*0201 molecule, an HLA A*0101 molecule, an HLA A*0301 molecule, an HLA B*4402 molecule, an HLA B*0702 molecule, an HLA B*2705 molecule, an HLA *A1101 molecule, an HLA *A2301 molecule, an HLA *A2402 molecule, an HLA *B0801 molecule, an HLA *B1401 molecule, an HLA *B1402 molecule, an HLA *B1501 molecule, an HLA *B1503 molecule, an HLA *B1510 molecule, an HLA *B1511 molecule, an HLA *B1518 molecule, an HLA *B4001 molecule, an HLA *B4901 molecule, an HLA *C0303 molecule, an HLA *C0304 molecule, an HLA *C0501 molecule, an HLA *0602 molecule, an HLA *0701 molecule, an HLA *0702 molecule, and an HLA *0704 molecule. The compositions can in some embodiments have at least one phosphopeptide specific for an HLA allele selected from the group consisting of HLA-A*0201 molecule, an HLA A*0101 molecule, an HLA A*0301 molecule, an HLA B*4402 molecule, an HLA B*0702 molecule, an HLA B*2705 molecule, an HLA *A1101 molecule, an HLA *A2301 molecule, an HLA *A2402 molecule, an HLA *B0801 molecule, an HLA *B1401 molecule, an HLA *B1402 molecule, an HLA *B1501 molecule, an HLA *B1503 molecule, an HLA *B1510 molecule, an HLA *B1511 molecule, an HLA *B1518 molecule, an HLA *B4001 molecule, an HLA *B4901 molecule, an HLA *C0303 molecule, an HLA *C0304 molecule, an HLA *C0501 molecule, an HLA *0602 molecule, an HLA *0701 molecule, an HLA *0702 molecule, and an HLA *0704 molecule. In some embodiments, the compositions can further comprise additional phosphopeptides from other MHC class I and/or class II alleles.

As such, the compositions of the presently disclosed subject matter containing various combinations of peptides will in some embodiments be immunologically suitable for between or about 3-88%, 80-89%, 70-79%, 60-69%, 57-59%, 55-57%, 53-55% or 51-53% or 5-90%, 10-80%, 15-75%, 20-70%, 25-65%, 30-60%, 35-55%, or 40-50% of the population of a particular cancer and/or a microbial infection. In some embodiments, the compositions of the presently disclosed subject matter are able to act as vaccine compositions for eliciting anti-tumor immune responses or in adoptive T-cell therapy of cancer patients and patients with microbial infections, wherein the compositions are immunologically suitable for about or at least 88, 87, 86, 85, 84, 83, 82, 81, 80, 79, 78, 77, 76,75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 20, 19, 18, 17, 16, 15, 14, 13, 12, 11, 10, 9, 8, 7, 6, 5, 4 or 3 percent of cancer patients and/or patients with microbial infections.

### IV. Compositions and Methods of Use

“Peptide compositions” as used herein refers to at least one peptide formulated for example, as a vaccine; or as a preparation for pulsing cells in a manner such that the pulsed cells, e.g., dendritic cells, will display the at least one peptide in the composition on their surface, e.g., to T-cells in the context of adoptive T-cell therapy.

The compositions of the presently disclosed subject matter can include in some embodiments about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50-55, 55-65, 65-80, 80-120, 90-150, 100-175, or 175-250 different peptides.

The compositions of the presently disclosed subject matter generally include MHC class I- and/or class II-specific peptide(s) but in some embodiments can also include one or more peptides specific for MHC class I and/or class II and/or other peptides associated with tumors, e.g., tumor-associated antigen (“TAA”).

Compositions comprising the presently disclosed peptide are typically substantially free of other human proteins or peptides. They can be made synthetically or by purification from a biological source. They can be made recombinantly. In some embodiments, they are at least 90%, 92%, 93%, 94%, at least 95%, or at least 99% pure. For administration to a human body, in some embodiments they do not contain other components that might be harmful to a human recipient. The compositions are typically devoid of cells, both human and recombinant producing cells. However, as noted below, in some cases, it can be desirable to load dendritic cells with a peptide and use those loaded dendritic cells as either an immunotherapy agent themselves, or as a reagent to stimulate a patient's T cells ex vivo. The stimulated T cells can be used as an immunotherapy agent. In some embodiments, it can be desirable to form a complex between a peptide and an HLA molecule of the appropriate type. Such complexes can in some embodiments be formed in vitro or in vivo. Such complexes are typically tetrameric with respect to an HLA-peptide complex. Under certain circumstances it can be desirable to add additional proteins or peptides, for example, to make a cocktail having the ability to stimulate an immune response in a number of different HLA type hosts. Alternatively, additional proteins or peptide can provide an interacting function within a single host, such as an adjuvant function or a stabilizing function. As a non-limiting example, other tumor antigens can be used in admixture with the peptides, such that multiple different immune responses are induced in a single patient.

Administration of peptides to a mammalian recipient can in some embodiments be accomplished using long peptides (e.g., longer than 8, 10, 12, or 15 residues) or using peptide-loaded dendritic cells (see Melief, 2009). The immediate goal is to induce activation of CD8⁺ T cells. Additional components which can be administered to the same patient, either at the same time or close in time (e.g., within 21 days of each other) include TLR-ligand oligonucleotide CpG and related peptides that have overlapping sequences of at least 6 amino acid residues. To ensure efficacy, mammalian recipients should express the appropriate human HLA molecules to bind to the peptides. Transgenic mammals can be used as recipients, for example, if they express appropriate human HLA molecules. If a mammal's own immune system recognizes a similar peptide then it can be used as model system directly, without introducing a transgene. Useful models and recipients can in some embodiments be at increased risk of developing metastatic cancer, such as HCC. Other useful models and recipients can be predisposed, e.g., genetically or environmentally, to develop HCC or other cancer.

IV.A. Selection of Peptides

Disclosed herein is the finding that immune responses can be generated against phosphorylated peptides tested in healthy and diseased individuals. The T-cells associated with these immune responses, when expanded in vitro, are able to recognize and kill malignant tissue (both established cells lines and primary tumor samples). Cold-target inhibition studies reveal that these peptide-specific T-cell lines kill primary tumor tissue in a peptide-specific manner.

When selecting peptides of the presently disclosed subject matter for inclusion in immunotherapy, e.g., in adaptive cell therapy or in the context of a vaccine, one can preferably pick peptides that in some embodiments: 1) are associated with a particular cancer/tumor cell type; 2) are associated with a gene/protein involved in cell proliferation; 3) are specific for an HLA allele carried the group of patients to be treated; and/or 4) are capable of inducing a peptide-specific memory T cell response in the patients to be treated upon a first exposure to a composition including the selected peptides.

IV.B. Peptide Vaccines

The peptides of the presently disclosed subject matter can also in some embodiments be used to vaccinate an individual. The peptides can be injected alone or in some embodiments can be administered in combination with an adjuvant, a pharmaceutically acceptable carrier, or combinations thereof. Vaccines are envisioned to prevent or treat certain diseases, disorders, and/or conditions in general, and cancers and/or microbial infections in particular.

The peptide compositions of the presently disclosed subject matter can in some embodiments be used as a vaccine for cancer, and more specifically for hepatocellular carcinoma (HCC), esophageal cancer, melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, and cervical cancer. The compositions can in some embodiments include peptides. The vaccine compositions can in some embodiments include only the peptides, or peptides disclosed herein, or they can include other cancer antigens that have been identified.

Additionally, compositions of the presently disclosed subject matter can in some embodiments be used as a vaccine for microbial infections.

The vaccine compositions can in some embodiments be used prophylactically for the purposes of preventing, reducing the risk of, and/or delaying initiation of a cancer and/or a microbial infection in an individual that does not currently have cancer. Alternatively, they can be used to treat an individual that already has cancer, so that recurrence or metastasis is delayed and/or prevented. Prevention relates to a process of prophylaxis in which the individual is immunized prior to the induction or onset of cancer. For example, individuals with a history of poor life style choices and at risk for developing HCC can in some embodiments be immunized prior to the onset of the disease.

Alternatively or in addition, individuals that already have cancer can be immunized with the antigens of the presently disclosed subject matter so as to stimulate an immune response that would be reactive against the cancer. A clinically relevant immune response would be one in which the cancer partially or completely regresses and/or is eliminated from the patient, and it would also include those responses in which the progression of the cancer is blocked without being eliminated. Similarly, prevention need not be total, but can in some embodiments result in a reduced risk, delayed onset, and/or delayed progression or metastasis.

The peptide vaccines of the presently disclosed subject matter can in some embodiments be given to patients before, after, or during any of the aforementioned stages of cancer and/or microbial infection. In some embodiments, they are given to patients with malignant HCC and/or malignant esophageal cancer (e.g., squamous cell carcinoma and/or adenocarcinoma).

In some embodiments, the 5-year survival rate of patients treated with the vaccines of the presently disclosed subject matter is increased by a statistically significant amount, e.g., by about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more percent, relative to the average 5-year survival rates described above.

In some embodiments, the peptide vaccine composition of the presently disclosed subject matter will increase survival rates in patients with cancer (e.g., metastatic HCC and/or malignant esophageal cancer) by a statistically significant amount of time, e.g., by about or at least, 0.25, 0.5, 0.75, 1.0, 1.25, 1.5, 1.75, 2.0, 2.25, 2.5, 2.75, 3.0, 3.25, 3.5, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.50, 9.75, 10.0, 10.25, 10.5, 10.75, 11.0, 11.25, 11.5, 11.75, or 12 months or more compared to what could have been expected without vaccine treatment at the time of filing of this disclosure.

In some embodiments, the survival rate, e.g., the 1, 2, 3, 4, or 5-year survival rate, of patients treated with the vaccines of the presently disclosed subject matter is increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, relative to the average 5-year survival rates described above.

The peptide vaccines of the presently disclosed subject matter are in some embodiments envisioned to illicit a T cell associated immune response, e.g., generating activated CD8⁺ T cells specific for native peptide/MHC class I expressing cells, specific for at least or about 1, 2, 3, 4, 5, 6, 7, 8, 9, 10, or more of the peptides in the vaccine in a patient for about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 98, 99, or 100 days after providing the vaccine to the patient.

In some embodiments, the treatment response rates of patients treated with the peptide vaccines of the presently disclosed subject matter are increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 98, 99, 100, 150, 200, 250, 300, 350, 400, 450, 500, or more percent, relative to treatment without the vaccine.

In some embodiments, overall median survival of patients treated with the peptide vaccines of the presently disclosed subject matter is increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, or more percent, relative to treatment without the vaccine. In some embodiments, the overall median survival of cancer patients and/or patients with microbial infections treated the peptide vaccines is envisioned to be about or at least 10.0, 10.25, 10.5, 10.75, 11.0, 11.25, 11.5, 11.75, 12, 12.25, 12.5, 12.75, 13, 13.25, 13.5, 13.75, 14, 14.25, 14.5, 14.75, 15, 16, 17, 18, 19, 20, 21, 22, 23, 24, 25, 26, 27, 28, 29, 30, 31, 32, 33, 34, 35, 36, or more months.

In some embodiments, tumor size of patients treated with the peptide vaccines of the presently disclosed subject matter is decreased by a statistically significant amount, e.g., by about, or by at least, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, or more percent, relative to treatment without the vaccine.

In some embodiments, the compositions of the presently disclosed subject matter provide an clinical tumor regression by a statistically significant amount, e.g., in about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of patients treated with a composition of the presently disclosed subject matter.

In some embodiments, the compositions of the presently disclosed subject matter provide a CTL response specific for the cancer being treated (such as but not limited to HCC and/or malignant esophageal cancer) and/or a microbial infection by a statistically significant amount, e.g., in about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of patients treated with a composition of the presently disclosed subject matter.

In some embodiments, the compositions of the presently disclosed subject matter provide an increase in progression free survival in the cancer being treated (e.g., HCC and/or malignant esophageal cancer), of about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500, or more percent compared to the progression free survival or patients not treated with the composition.

In some embodiments, progression free survival, CTL response rates, clinical tumor regression rates, tumor size, survival rates (including but not limited to overall survival rates), and/or response rates are determined, assessed, calculated, and/or estimated weekly, monthly, bi-monthly, quarterly, semi-annually, annually, and/or bi-annually over a period of about or at least 1, 2, 3, 4, 5, 6, 7, 8, 9, 10, 11, 12, 13, 14, 15 or more years or about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500, or more weeks.

IV.C. Compositions for Priming T cells

Adoptive cell transfer is the passive transfer of cells, in some embodiments immune-derived cells, into a recipient host with the goal of transferring the immunologic functionality and characteristics into the host. Clinically, this approach has been exploited to transfer either immune-promoting or tolerogenic cells (often lymphocytes) to patients to enhance immunity against cancer. The adoptive transfer of autologous tumor infiltrating lymphocytes (TIL) or genetically re-directed peripheral blood mononuclear cells has been used to successfully treat patients with advanced solid tumors, including melanoma and ovarian carcinoma, HCC, and/or malignant esophageal cancer (e.g., squamous cell carcinoma and/or adenocarcinoma), as well as patients with CD19-expressing hematologic malignancies. In some embodiments, adoptive cell transfer (ACT) therapies achieve T-cell stimulation ex vivo by activating and expanding autologous tumor-reactive T-cell populations to large numbers of cells that are then transferred back to the patient (see e.g., Gattinoni et al., 2006).

The peptides of the presently disclosed subject matter can in some embodiments take the form of antigen peptides formulated in a composition added to autologous dendritic cells and used to stimulate a T helper cell or CTL response in vitro. The in vitro generated T helper cells or CTL can then be infused into a patient with cancer (Yee et al., 2002), and specifically a patient with a form of cancer that expresses one or more of antigen peptides.

Alternatively or in addition, the peptides of the presently disclosed subject matter can be added to dendritic cells in vitro, with the loaded dendritic cells being subsequently transferred into an individual with cancer in order to stimulate an immune response. Alternatively or in addition, the loaded dendritic cells can be used to stimulate CD8⁺ T cells ex vivo with subsequent reintroduction of the stimulated T cells to the patient. Although a particular peptide can be identified on a particular cancer cell type, it can be found on other cancer cell types.

The presently disclosed subject matter envisions treating cancer by providing a patient with cells pulsed with a composition of peptides. The use of dendritic cells (“DCs”) pulsed with peptide antigens allows for manipulation of the immunogen in two ways: varying the number of cells injected and varying the density of antigen presented on each cell. Exemplary methods for DC-based based treatments can be found for example in Mackensen et al., 2000.

IV.D. Additional Peptides Present in Peptide Compositions

The peptide compositions (or peptide composition kits) of the presently disclosed subject matter can in some embodiments also include at least one additional peptide derived from tumor-associated antigens. Examples of tumor-associated antigens include MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, prostatic acid phosphatase, and the like. Particular examples of additional peptides derived from tumor-associated antigens that can be employed alone or in combination with the compositions of the presently disclosed subject matter those set forth in Table 2 below.

TABLE 2
Exemplary Peptides Derived from Tumor-associated Antigens

| Polypeptide | Amino Acid Sequence^{b} | GENBANK® |
|---|---|---|
| Name^{a} | (SEQ ID NO:) | Acc. No(s).^{c} |
| CEA₆₁₋₆₉ | HLFGYSWYK (SEQ ID NO: 3924) | NP_001264092.1 |
| | | XP_005278431.1 |
| CEA₆₀₄₋₆₁₂ | YLSGADLNL (SEQ ID NO: 3925) | XP_005278431.1 |
| FBP/FOLR1₁₉₁₋₁₉₉ | EIWTHSYKV (SEQ ID NO: 3926) | NP_000793.1 |
| gp100₁₇₋₂₅ | ALLAVGATK (SEQ ID NO: 3927) | NP_001186982.1 |
| gp100₄₄₋₅₉ | WNRQLYPEWTEAQRLD | NP_008859.1 |
| | (SEQ ID NO: 3928 | |
| gp100₈₇₋₉₅ | ALNFPGSQK (SEQ ID NO: 3929) | NP_008859.1 |
| gp100₈₉₋₉₅ | SQNFPGSQK (SEQ ID NO: 3930) | NP_008859.1 |
| gp100₁₅₄₋₁₆₂ | KTWGQYWQV (SEQ ID NO: 3931_ | NP_008859.1 |
| gp100₂₀₉₋₂₁₇ | ITDQVPFSV (SEQ ID NO: 3932) | NP_008859.1 |
| gp100₂₀₉₋₂₁₇ | IMDQVPFSV (SEQ ID NO: 3933 | NP_008859.1 |
| gp100₂₈₀₋₂₈₈ | YLEPGPVTA (SEQ ID NO: 3934) | NP_008859.1 |
| gp100₄₇₆₋₄₈₅ | VLYRYGSFSV (SEQ ID NO: 3935) | NP_008859.1 |
| gp100₆₁₄₋₆₂₂ | LIYRRRLMK (SEQ ID NO: 3936) | NP_008859.1 |
| Her2/neu₃₆₉₋₃₇₇ | KIFGSLAFL (SEQ ID NO: 3937) | NP_004439.2 |
| Her2/neu₇₅₄₋₇₆₂ | VLRENTSPK (SEQ ID NO: 3938) | NP_004439.2 |
| MAGE-A1₁₁₄₋₁₂₇ | LLKYRAREPVTKAE | NP_004979.3 |
| MAGE-A2, 3, 6₁₂₁₋₁₃₄ | (SEQ ID NO: 3939) | NP_005352.1 |
| | | NP_005353.1 |
| | | NP_005354.1 |
| MAGE-A1₉₆₋₁₀₄ | SLFRAVITK (SEQ ID NO: 3940) | NP_004979.3 |
| MAGE-A1₁₆₁₋₁₆₉ | EADPTGHSY (SEQ ID NO: 3941) | NP_004979.3 |
| MAGE-A3₁₆₈₋₁₇₆ | EVDPIGHLY (SEQ ID NO: 3942) | NP_005353.1 |
| MAGE-A3₂₈₁₋₂₉₅ | TSYVKVLHHMVKISG | NP_005353.1 |
| | (SEQ ID NO: 3943) | |
| MAGE-A10₂₅₄₋₂₆₂ | GLYDGMEHL (SEQ ID NO: 3944) | NP_001011543.2 |
| MART-1/MelanA₂₇₋₃₅ | AAGIGILTV (SEQ ID NO: 3945) | NP_005502.1 |
| MART-1/MelanA₅₁₋₇₃ | RNGYRALMDKSLHVGTQCALTRR | NP_005502.1 |
| | (SEQ ID NO: 3946) | |
| MART-1/MelanA₉₇₋₁₁₆ | VPNAPPAYEKLsAEQSPPPY | NP_005502.1 |
| | (SEQ ID NO: 3947) | |
| MART-1/MelanA₉₈₋₁₀₉ | PNAPPAYEKLsA (SEQ ID NO: 3948) | NP_005502.1 |
| MART-1/MelanA₉₉₋₁₀₀ | NAPPAYEKLsAE (SEQ ID NO: 3949) | NP_005502.1 |
| MART-1/MelanA₁₀₀₋₁₀₈ | APPAYEKLs (SEQ ID NO: 3950) | NP_005502.1 |
| MART-1/MelanA₁₀₀₋₁₁₁ | APPAYEKLsAEQ (SEQ ID NO: 3951) | NP_005502.1 |
| MART-1/MelanA₁₀₀₋₁₁₄ | APPAYEKLsAEQSPP | NP_005502.1 |
| | (SEQ ID NO: 3952) | |
| MART-1/MelanA₁₀₀₋₁₁₅ | APPAYEKLsAEQSPPP | NP_005502.1 |
| | (SEQ ID NO: 3953) | |
| MART-1/MelanA₁₀₀₋₁₁₆ | APPAYEKLsAEQSPPPY | NP_005502.1 |
| | (SEQ ID NO: 3954) | |
| MART-1/MelanA₁₀₁₋₁₀₉ | PPAYEKLsA (SEQ ID NO: 3955) | NP_005502.1 |
| MART-1/MelanA₁₀₁₋₁₁₂ | PPAYEKLsAEQS (SEQ ID NO: 3956) | NP_005502.1 |
| MART-1/MelanA₁₀₂₋₁₁₀ | PAYEKLsAE (SEQ ID NO: 3957) | NP_005502.1 |
| MART-1/MelanA₁₀₂₋₁₁₃ | PAYEKLsAEQSP (SEQ ID NO: 3958) | NP_005502.1 |
| MART-1/MelanA₁₀₃₋₁₁₄ | AYEKLsAEQSPP (SEQ ID NO: 3959) | NP_005502.1 |
| MART-1/MelanA₁₀₄₋₁₁₅ | YEKLsAEQSPPP (SEQ ID NO: 3960) | NP_005502.1 |
| NY-ESO-1 | AAQERRVPR (SEQ ID NO: 3961) | AAD05203.1 |
| | | CAA10193.1 |
| NY-ESO-1 | LLGPGRPYR (SEQ ID NO: 3962) | NP_001913.2 |
| NY-ESO-1₅₃₋₆₂ | ASGPGGGAPR (SEQ ID NO: 3963) | NP_001318.1 |
| p2₈₃₀₋₈₄₄ | AQYIKANSKFIGITEL | NP_783831.1 |
| | (SEQ ID NO: 3964) | |
| TAG-1,2 | RLSNRLLLR (SEQ ID NO: 3965) | |
| Tyr₅₆₋₇₀ | AQNILLSNAPLGPQFP | NP_000363.1 |
| | (SEQ ID NO: 3966) | |
| Tyr₁₄₆₋₁₅₆ | SSDYVIPIGTY (SEQ ID NO: 3967) | NP_000363.1 |
| Tyr₂₄ₒ₋₂₅ᵢ | SDAEKSDICTDEY | NP_000363.1 |
| | (SEQ ID NO: 3968) | |
| Tyr₂₄₃₋₂₅₁ | KCDICTDEY (SEQ ID NO: 3969) | NP_000363.1 |
| Tyr₃₆₉₋₃₇₇ | YMDGTMSQV (SEQ ID NO: 3970) | NP_000363.1 |
| Tyr₃₈₈₋₄₀₆ | FLLHHAFVDSIFEQWLQRHRP | NP_000363.1 |
| | (SEQ ID NO: 3971) | |

^{a}Numbers listed in subscript are the amino acids positions of the listed peptide sequence in the corresponding polypeptide including, but not limited to the amino acid sequences provided in the GENBANK® biosequence database.
^{b}lower case amino acids in this column are optionally phosphorylated.
^{c}GENBANK® biosequence database Accession Numbers listed here are intended to be exemplary only and should not be interpreted to limit the disclosed peptide sequences to only these polypeptides.

Such tumor specific peptides (including the WIC class I phosphopeptides disclosed in Tables 3-7 can be added to the peptide compositions in a manner, number, and/or in an amount as if they were an additional peptide added to the peptide compositions as described herein.

IV.E. Combination Therapies

In some embodiments, the peptide compositions (or peptide composition kits) of the presently disclosed subject matter are administered as a vaccine or in the form of pulsed cells as first, second, third, or fourth line treatment for the cancer and/or microbial infection. In some embodiments, the compositions of the presently disclosed subject matter are administered to a patient in combination with one or more therapeutic agents, e.g., anti-CA125 (or oregovomab Mab B43.13), anti-idiotype Ab (ACA-125), anti-HER-2 (trastuzumab, pertuzumab), anti-MUC-1 idiotypic Ab (HMFG1), HER-2/neu peptide, NY-ESO-1, anti-Programed Death-1 (“PD1”) (or PD1-antagonists such as BMS-936558), anti-CTLA-4 (or CTLA-4 antagonists), vermurafenib, ipilimumab, dacarbazine, IL-2, IFN-α, IFN-γ, temozolomide, receptor tyrosine kinase inhibitors (e.g., imatinib, gefitinib, erlotinib, sunitinib, tyrphostins, telatinib), sipileucel-T, tumor cells transfected with GM-CSF, a platinum-based agent, a taxane, an alkylating agent, an antimetabolite and/or a vinca alkaloid or combinations thereof. In an embodiment, the cancer is sensitive to or refractory, relapsed or resistant to one or more chemotherapeutic agents, e.g., a platinum-based agent, a taxane, an alkylating agent, an anthracycline (e.g., doxorubicin (e.g., liposomal doxorubicin)), an antimetabolite and/or a vinca alkaloid. In some embodiments, the cancer is, e.g., HCC, and the HCC is refractory, relapsed, or resistant to a platinum-based agent (e.g., carboplatin, cisplatin, oxaliplatin), a taxane (e.g., paclitaxel, docetaxel, larotaxel, cabazitaxel) and/or an anthracycline (e.g., doxorubicin (e.g., liposomal doxorubicin)). In some embodiments, the cancer is, e.g., HCC, and the HCC is refractory, relapsed, or resistant to an antimetabolite (e.g., an antifolate (e.g., pemetrexed, floxuridine, raltitrexed) and a pyrimidine analogue (e.g., capecitabine, cytrarabine, gemcitabine, 5FU)) and/or a platinum-based agent (e.g., carboplatin, cisplatin, oxaliplatin). In some embodiments, the cancer is, e.g., lung cancer, and the cancer is refractory, relapsed or resistant to a taxane (e.g., paclitaxel, docetaxel, larotaxel, cabazitaxel), a platinum-based agent (e.g., carboplatin, cisplatin, oxaliplatin), a vinca alkaloid (e.g., vinblastine, vincristine, vindesine, vinorelbine), a vascular endothelial growth factor (VEGF) pathway inhibitor, an epidermal growth factor (EGF) pathway inhibitor) and/or an antimetabolite (e.g., an antifolate (e.g., pemetrexed, floxuridine, raltitrexed) and a pyrimidine analogue (e.g., capecitabine, cytrarabine, gemcitabine, 5FU)). In some embodiments, the cancer is, e.g., breast cancer, and the cancer is refractory, relapsed or resistant to a taxane (e.g., paclitaxel, docetaxel, larotaxel, cabazitaxel), a vascular endothelial growth factor (VEGF) pathway inhibitor, an anthracycline (e.g., daunorubicin, doxorubicin (e.g., liposomal doxorubicin), epirubicin, valrubicin, idarubicin), a platinum-based agent (e.g., carboplatin, cisplatin, oxaliplatin), and/or an antimetabolite (e.g., an antifolate (e.g., pemetrexed, floxuridine, raltitrexed) and a pyrimidine analogue (e.g., capecitabine, cytrarabine, gemcitabine, 5FU)). In some embodiments, the cancer is, e.g., gastric cancer, and the cancer is refractory, relapsed or resistant to an antimetabolite (e.g., an antifolate (e.g., pemetrexed, floxuridine, raltitrexed) and a pyrimidine analogue (e.g., capecitabine, cytrarabine, gemcitabine, 5FU)) and/or a platinum-based agent (e.g., carboplatin, cisplatin, oxaliplatin). In some embodiments, an antimicrobial and/or an antiviral is administered to the patient.

In some embodiments, the peptide compositions (or peptide composition kits) of the presently disclosed subject matter are associated with agents that inhibit T cell apoptosis or anergy thus potentiating a T cell response (“T cell potentiator”). Such agents include B7RP1 agonists, B7-H3 antagonists, B7-H4 antagonists, HVEM antagonists, HVEM antagonists, GALS antagonists or alternatively CD27 agonists, OX40 agonists, CD137 agonists, BTLA agonists, ICOS agonists CD28 agonists, or soluble versions of PDL1, PDL2, CD80, CD96, B7RP1, CD137L, OX40 or CD70. See Pardoll, 2012.

In some embodiments, the T cell potentiator is a PD1 antagonist. Programmed death 1 (PD1) is a key immune checkpoint receptor expressed by activated T cells, and it mediates immunosuppression. PD1 functions primarily in peripheral tissues, where T cells can encounter the immunosuppressive PD1 ligands PD-L1 (B7-H1) and PD-L2 (B7-DC), which are expressed by tumor cells, stromal cells, or both. In some embodiments, the anti-PD1 monoclonal antibody BMS-936558 (also known as MDX-1106 and ONO-4538) is used. In some embodiments, the T cell potentiator, e.g., PD1 antagonist, is administered as an intravenous infusion at least or about every 1, 1.5, 2, 2.5, 3, 3.5, or 4 weeks of each 4, 5, 6, 7, 8, 9, or 10-week treatment cycle of about for at least 1, 2, 3, 4, 5, 6, 7, 8, 9, 10, 11, 12, 13, 14, 15, 16, 17, 18, 19, 20, or more cycles. Exemplary, non-limiting doses of the PD1 antagonists are envisioned to be exactly, about, or at least 0.1, 0.2, 0.3, 0.4, 0.5, 0.6, 0.7, 0.8, 0.9, 1, 2, 3, 4, 5, 6, 7, 8, 9, 10, 11, 12, 13,14, 15, 16, 17, 18, 19, 20, or more mg/kg (see Brahmer et al., 2012).

The exemplary therapeutic agents disclosed herein above are envisioned to be administered at a concentration of, e.g., about 1 to 100 mg/m², about 10 to 80 mg/m², about 40 to 60 mg/m², e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more mg/mm². Alternatively, the exemplary therapeutic agents disclosed herein above are envisioned to be administered at a concentration of, e.g., about or at least 0.001 to 100 mg/kg or 0.1 to 1 mg/kg. In some embodiments, the exemplary therapeutic agents disclosed herein above are envisioned to be administered at a concentration of, e.g., about or at least from 0.01 to 10 mg/kg.

The peptide compositions (or peptide composition kits) of the presently disclosed subject matter can in some embodiments also be provided with administration of cytokines such as lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha -beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

The peptide compositions of the presently disclosed subject matter can in some embodiments be provided with administration of cytokines around the time, (e.g., about or at least 1, 2, 3, or 4 weeks or days before or after) of the initial dose of a peptide composition.

Exemplary, non-limiting doses of a cytokine would be about or at least 1-100, 10-80, 20-70, 30-60, 40-50, or 1, 2, 3, 4, 5, 6, 7, 8, 9, or 10 Mu/m²/day over about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. The cytokine can in some embodiments be delivered at least or about once every 1, 2, 3, 4, 5, 6, 7, 8, 9, 10, 11, 12, 13, 14, 15, 16, 17, 18, 19, 20, 21, 22, 23, or 24 hours. Cytokine treatment can in some embodiments be provided in at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cycles of at least or about 1, 2, 3, 4, 5, 6, 7, 8, 9, or 10 weeks, wherein each cycle has at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cytokine doses. Cytokine treatment can be on the same schedule as administration of the peptide compositions or on a different (but in some embodiments overlapping) schedule.

In some embodiments, the cytokine is IL-2 and is dosed in an amount of about or at least 100,000 to 1,000,000; 200,000-900,000; 300,000-800,000; 450,000-750,000; 600,000-800,000; or 700,000-800,000; or 720,000 units (IU)/kg administered, e.g., as a bolus, every 1, 2, 3, 4, 5, 6, 7, 8, 9, 10, 11, 12, 13, 14, 15, 16, 17, 18, 19, or 20 hours for 1, 2, 3, 4, 5, 6, 7, 8, 9, 10, 11, 12, 13, or 14 days, in a cycle, for example.

### V. Types of Diseases, Disorders, and Conditions

The compositions of the presently disclosed subject matter are envisioned to useful in the treatment of benign and malignant proliferative diseases and microbial infections. Excessive proliferation of cells and turnover of cellular matrix can contribute significantly to the pathogenesis of several diseases, including but not limited to cancer, atherosclerosis, rheumatoid arthritis, psoriasis, idiopathic pulmonary fibrosis, scleroderma and cirrhosis of the liver, ductal hyperplasia, lobular hyperplasia, papillomas, and others.

In some embodiments, the proliferative disease is cancer, which in some embodiments is selected from the group consisting of HCC, esophageal cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer. In some embodiments, the compositions of the presently disclosed subject matter are used to treat HCC, esophageal cancer, colorectal cancer, acute myelogenous leukemia (AML), acute lymphocytic leukemia (ALL), chronic lymphocytic lymphoma (CLL), chronic myelogenous leukemia (CML), breast cancer, renal cancer, pancreatic cancer, and/or ovarian cancer.

In some embodiments, the cancer is a cancer of the bladder (including accelerated and metastatic bladder cancer), breast (e.g., estrogen receptor positive breast cancer, estrogen receptor negative breast cancer, HER-2 positive breast cancer, HER-2 negative breast cancer, triple negative breast cancer, inflammatory breast cancer), colon (including colorectal cancer), kidney (e.g., renal cell carcinoma), liver, lung (including small cell lung cancer and non-small cell lung cancer (including adenocarcinoma, squamous cell carcinoma, bronchoalveolar carcinoma and large cell carcinoma)), genitourinary tract, e.g., ovary (including fallopian, endometrial and peritoneal cancers), cervix, prostate and testes, lymphatic system, rectum, larynx, pancreas (including exocrine pancreatic carcinoma), stomach (e.g., gastroesophageal, upper gastric or lower gastric cancer), gastrointestinal cancer (e.g., anal cancer), gall bladder, thyroid, lymphoma (e.g., Burkitt's, Hodgkin's, or non-Hodgkin's lymphoma), leukemia (e.g., acute myeloid leukemia), Ewing's sarcoma, nasoesophageal cancer, nasopharyngeal cancer, neural and glial cell cancers (e.g., glioblastoma multiforme), and head and neck. Exemplary cancers include but are not limited to HCC, esophageal cancer (including Barrett's esophagus (BE), high-grade dysplasia (HGD), and invasive cancer including but not limited to squamous cell carcinoma and adenocarcinoma), melanoma, breast cancer (e.g., metastatic or locally advanced breast cancer), prostate cancer (e.g., hormone refractory prostate cancer), renal cell carcinoma, lung cancer (e.g., small cell lung cancer and non-small cell lung cancer (including adenocarcinoma, squamous cell carcinoma, bronchoalveolar carcinoma and large cell carcinoma)), pancreatic cancer, gastric cancer (e.g., gastroesophageal, upper gastric or lower gastric cancer), colorectal cancer, squamous cell cancer of the head and neck, ovarian cancer (e.g., advanced ovarian cancer, platinum-based agent resistant or relapsed ovarian cancer), lymphoma (e.g., Burkitt's, Hodgkin's, or non-Hodgkin's lymphoma), leukemia (e.g., acute myeloid leukemia), and gastrointestinal cancer.

In some embodiments, the compositions and methods of the presently disclosed subject matter are for use in treating microbial infections. Exemplary microbes that can be treated with the compositions and methods of the presently disclosed subject matter include at least the following:

Hepatitis C and B viruses. Worldwide, there are 140 million and more than 250 million people chronically infected with hepatitis C virus (HCV) and hepatitis B virus, (HBV), respectively. Both viruses can cause hepatocellular cancer. HCV consists of a single stranded RNA (9600 nucleotide bases) surrounded by a protected shell of proteins. The viral RNA codes for a single polyprotein (˜3,000 AA) that is post-translationally cleaved into two highly glycosylated structural proteins, E1 and E2, a transmembrane protein p7, and six non-structural accessory proteins, NS2, NS3, NS4A, NS4B, NS5A, and NS5B.

HCV does not integrate its genome into the host chromosomal DNA. It does exhibit a high mutational rate and does deregulate many host cellular processes. Accessory protein NS5B forms a complex with the retinoblastoma tumor suppressor protein (pRb) that is then targeted for degradation in the proteasome following ubiquitination by the E6-associated protein (E6AP). Expression of another member of the pRb family, p130, is downregulated by HCV core protein that triggers hyper-methylation of the promoter region of the corresponding gene. Accessory protein NS2 sequesters p53 to the cytoplasm and prevents it from monitoring DNA damage and triggering cell apoptosis. The expected result would be high levels of gene transcription including likely production of cancerous inhibitor of PP2A (CIP2A; also called cellular inhibitor of PP2A) and uncontrolled cell division. Partially at odds with this expectation are data that suggest a third accessory protein, NS5A, functions as a PP2A regulatory protein that enhances a particular PP2A activity and partially reduces protein phosphorylation.

The hepatitis B virus (HBV) is a partially double-stranded DNA virus that replicates via reverse transcription. The two DNA chains contain ˜3200 and ˜2300 nucleotides, respectively. The genome contains four overlapping reading frames that code for the viral coat protein (capsid), surface proteins (envelope), reverse transcriptase, and the small (17.4 kDa), regulatory oncoprotein, HBx. Integration of HBV into the host hepatocyte genome is a frequent event in HCC (86.4%). HBx activates the E2F1 group of transcription factors by upregulating kinases that phosphorylate and inactivate pRb. The result is high levels of transcription and likely generation of the PP2A inhibitor CIP2A. A number of reports also indicate that HBx blocks apoptosis of HBV infected cells by several different mechanisms. Since PP2A is largely inhibited by both viruses, as disclosed herein many of the same class I MHC phosphopeptide antigens that have been identified on multiple cancers have also been identified on HCV- and HBV-infected cells.

Human Papillomavirus, HPV. Human papillomavirus (HPV) infects the basal cells of human epithelia and is the main causative agent for a large number of human tumors including cervical, head and neck, plus oral cancers. Although close to 200 different HPV types have been described, two variants, HPV-16 and HPV-18, are the types most often found in cervical cancer, the second most common cancer in women worldwide. The HPV-16 and 18 variants contain a small, double stranded DNA that encodes six regulatory proteins, (E1, E2, E4, E5, E6, and E7) and two structural proteins (L1 and L2). The initial stage of the infection occurs in the basal layer of undifferentiated epithelial cells and the virus is confined to the cell nucleus as an episome (host and viral DNA remain separate). Viral replication, facilitated by E1 and E2 and the host machinery, occurs at a slow rate without cell lysis or inflammation to avoid detection by the immune system.

To keep the cellular replication machinery active, the virus employs three of the other accessary proteins, E5, E6, and E7. All are oncogenic and of particular interest because of the roles they play in cancer development. E7 is a 98 residue phosphoprotein that binds to the active, unphosphorylated form of pRb (plus related proteins p130 and p107) and targets them for degradation in the proteasome. Active pRb binds and inactivates the E2F1-3 family of transcription factors and thus keeps the cell in a quiescent state. In the absence of pRb, the cell is free to undergo uncontrolled growth and proliferation. The accessary protein, E6, upregulates the DNA cytosine deaminase, APOBEC3B (A3B), an enzyme that converts cytosine to uracil and causes hypermutation of the viral DNA. Normally, this would activate the tumor suppressor protein, p53, to trigger apoptosis. Unfortunately, the 158 residue HPV E6 accessory protein and a cellular protein, E6AP, form a complex that allows them to bind p53 and target it for ubiquitination and degradation in the proteasome. During this period of the infection, multiple copies of the viral DNA that encode the oncoproteins, E6 and E7, become integrated into the host genome and replicate independently of the virus.

The third HPV accessary oncoprotein, E5, is a small 83 residue protein that localizes primarily to the endoplasmic reticulum and Golgi apparatus and plays a key role in regulating important growth factors and other proteins involved in control of cell differentiation, survival and growth. E5 also down regulates expression of class I and class II MHC molecules. Early studies concluded that the E5 protein is responsible for lack of acidification of the Golgi apparatus and for binding and prevention of class I molecules being transported to the cell surface. HPV-16 E5 was shown to selectively downregulate HLA-A and HLA-B presentation but had no effect on HLA-C and E molecules. Fortunately, viral DNA for the E5 oncoprotein is usually not incorporated into the host genome. As a result, levels of this protein in the transformed cells are expected to be much less than in the cells of the initial infection.

Note that when the E7 protein targets pRb for degradation, E2F1, a member of the E2F1-3 transcription factor family that was repressed by pRb, now becomes activated and upregulates expression of CIP2A. Inhibition of PP2A would thus be expected to dramatically increase the level and lifetime of phosphorylated proteins in the diseased cell and thus give rise to enhanced presentation of disease-specific, class I MHC phosphopeptides. Many of these phosphopeptides are expected to be the same as those that we have already identified on HLA A, B, and C alleles expressed on multiple types of cancer cells.

Epstein Barr Virus (EBV). More than 90% of adults in the world have been infected with the Epstein Barr Virus (EBV; also known as human herpesvirus 4, (HHV-4)) and most continue to have a lifelong dormant infection. EBV infects both B cells and epithelial cells. The reservoir for the latent virus is primarily resting, central memory, B-cells. EBV is known to cause infectious mononucleosis as well as a variety of cancers such as Hodgkin's lymphoma, Burkitt's lymphoma, gastric cancer, and nasopharyngeal carcinoma.

The virus is composed of a double DNA helix that codes for 85 proteins and is surrounded by a protein nucleocapsid and an envelope of both lipids and glycoproteins. Regulatory proteins of note include six nuclear antigens (EBNA-1, -2, -3A, -3B, 3C and the EBV nuclear antigen-leader protein EBNA-LP), plus three EBV latent membrane proteins (LMP-1, -2A, and -2B). EBNA-3C (also known as EBNA-6) binds the mitochondrial ribosomal protein MRPS18-2 and targets it to the nucleus where it binds to pRb and liberates the E2F1 group of transcription factors. EBNA-3C can also recruit the SCF^{Skp2} ubiquitin ligase complex which then mediates ubiquitination and degradation of pRb. High levels of transcription result. EBNA-3C also enhances the intrinsic ubiquitin ligase activity of Mdm2 toward p53, which in turn facilitates p53 ubiquitination and degradation.

Here as well, presentation of class I MHC phosphopeptides on the cell surface can result from targeting of pRb and p53 for degradation in the proteasome in order to liberate transcription factors that upregulate expression of PP2A protein inhibitors (e.g., SET and CIP2A). These inhibitors dramatically enhance the lifetime of phosphorylated proteins so that they can be degraded in the proteasome and unique phosphopeptide antigens can be presented on the cell surface by class I MHC molecules. When the immune system uses these antigens to defeat the virus, EBV is eliminated or becomes dormant, and memory T-cells are generated that can recognize other virus infections or cancer that express the same phosphopeptide antigens.

Merkel Cell Polyomavirus (MCPyV). MCPyV has a small (5,387 bp) double stranded DNA genome that codes for two viral coat proteins (VP1 and VP2) and four accessary proteins including a large tumor antigen (LT) and small tumor antigen (ST). The virus is the causative agent for Merkel cell carcinoma (MCC), a highly aggressive but rare skin cancer. Estimated cases of MCC per year number about 16,000. Most tumors are detected in the elderly or immunocompromised patients and are found on the head and neck area where the virus and skin are exposed to ultraviolet radiation. MCC results when viral DNA encoding ST and a mutated/truncated version of LT are incorporated into and expressed by the host genome.

This truncated version of LT is missing its DNA binding and growth suppressor domains but still contains the LXCXE motif that allows it to bind and inactivate pRb. This allows the cell to undergo uncontrolled proliferation. Full-length MCPyV LT represses transcription of p53 and thus blocks apoptosis. MCPyV ST displaces the regulatory protein B56a from active PP2A and likely competes with other regulatory B subunits for assembly of the intact holoenzyme. Again, these conditions are expected to result in the presentation of class I MHC phosphopeptide antigens that have already been observed on multiple cancers.

In addition, it is noted that MCPyV ST up-regulates glycolytic and metabolite transport genes including the major monocarboxylate transporter SLC16A1. This causes cells to convert pyruvate to lactate resulting in aerobic glycolysis, known as the Warburg effect. Generation of disease specific O-GlcNAcylated class I MHC peptides is predicted to result from this phenomenon, this type of class I MHC peptide antigen has been shown to be capable of generating strong memory T-cell responses in healthy blood donors.

Human Immunodeficiency Virus (HIV-1). HIV-1 is a retrovirus that infects CD4⁺ T-cells (T-helper cells), macrophages, and dendritic cells, eventually leading to the development of AIDS. More than 40 million people worldwide are infected with the virus.

HIV-1 is composed of two copies of single stranded RNA that codes for 16 proteins. Four HIV coded accessory proteins, Vif, Vpr, Nef, and Vpu, share the ability to target cellular proteins for proteasomal degradation and are essential for pathogenesis in vivo. Particularly relevant here is the recent discovery that the accessory protein Vif is necessary and sufficient for culin-5 (CUL5)-dependent ubiquitination and proteasomal degradation of all members of the B56 family of regulatory subunits (PPP2R-A, -B, -C, -D, and -E) of PP2A. Inhibition of PP2A by Vif produced hyperphosphorylation of cellular proteins that mirrored previously reported changes seen when PP2A in transformed cells was treated with the small molecule inhibitor okadaic acid. These observations suggest that HIV-1 infected cells should present numerous class I MHC phosphopeptide antigens.

Another HIV accessory protein, Nef, is known to subvert the host cellular trafficking machinery and to mediate down regulation of Class I/II MHC presentation on HIV infected cells. Rate of progression to AIDS seems to correlate with the extent of down regulation of MHC presentation. Since removal of all class I MHC proteins from the cell surface would expose the infected cell to attack by natural killer (NK) cells, the HIV virus has evolved to only suppress presentation of class I HLA-A and HLA-B proteins. Results of another study indicate that Nef is much more effective at suppression of HLA-A alleles than it is for HLA-B alleles. Presentation of HLA-C and E is not affected.

It is thus expected that class I MEW phosphopeptides presented by HLA A, B, and C alleles on cell lines that have been infected with HIV-1 could reflect data that has already been generated from the same alleles on multiple cancers.

Coronavirus. There are seven types coronaviruses (CoV) that can infect humans. Of particular interest are MERS-CoV (the beta coronavirus that causes Middle East Respiratory Syndrome, or MERS), SARS-CoV (the beta coronavirus that causes severe acute respiratory syndrome, or SARS), and SARS-CoV-2 (the novel coronavirus that causes coronavirus disease 2019, or COVID-19). The genome of SARS-CoV encodes the protein Nsp15 that has been shown to bind to and inhibit pRb1. This is expected to result in enhanced expression of CIP2A leading to high level expression of class I MEW phosphopeptides on viral infected cells. SARS-CoV-2's genome also encodes a Nsp15 protein and its amino acid sequence is 89% the same as that for corresponding SARS-CoV protein. As such, class I MHC phosphopeptides are expected to be expressed on coronavirus-infected cells, including cells infected with MERS-CoV, SARS-CoV, and SARS-CoV-2.

Helicobacter Pylori Bacterium (H. pylori). H. pylori is a gram-negative bacteria that colonizes the gastric epithelium and causes gastric cancer. Today, the disease is responsible for 700,000 deaths/year. About half the people in the world are presently infected with H. pylori but only a small percentage of the population ends up with cancer. Particularly virulent strains of the virus all code for the 120-140 kDa accessary protein, CagA, that can be translocated into host cells during bacterial attachment. CagA is phosphorylated on certain pentapeptide sequences near the C-terminus and can then recruit 20 of more binding partners and disrupt numerous signaling pathways in the host cell. CagA binds to E-cadherin and displaces β-catenin that then upregulates transcription in the host cell. This is expected to result in overexpression of CIP2A, high levels of long lived protein phosphorylation, and presentation of phosphopeptides on the surface of infected cells.

Fusobacterium nucleatum (Fn). Fusobacterium nucleatum (Fn) is a gram negative anaerobe that is usually found in the oral cavity and plays a key role in the development of dental plaque. Unfortunately, it also flourishes outside the oral cavity and is responsible for many infections. It is also known to promote colorectal carcinogenesis by modulating E-cadherin/β-catenin signaling. The Fn genome codes for a protein, FadA, that binds to E-cadherin on colorectal cells and mediates attachment and invasion of the bacterium. Both FadA and the Fn lipopolysaccharide have been reported to activate β-catenin signaling that upregulates transcription. This results in upregulation of CIP2A and inhibition of PP2A, resulting in high levels of phosphorylated proteins with long half-lives. Accordingly, the same phosphopeptide antigens that have been observed on multiple cancers would be expected to presented on Fn infected cells.

### VI. Administration of Compositions

The peptide compositions of the presently disclosed subject matter can in some embodiments be administered parenterally, systemically, and/or topically. By way of example and not limitation, composition injection can be performed by intravenous (i.v).

injection, sub-cutaneous (s.c). injection, intradermal (i.d). injection, intraperitoneal (i.p). injection, and/or intramuscular (i.m). injection. One or more such routes can be employed. Parenteral administration can be, for example, by bolus injection or by gradual perfusion over time. Alternatively or concurrently, administration can be by the oral route.

In some embodiments, intradermal (i.d). injection is employed. The peptide compositions of the presently disclosed subject matter are suitable for administration of the peptides by any acceptable route such as oral (enteral), nasal, ophthal, or transdermal. In some embodiments, the administration is subcutaneous and can be administered by an infusion pump.

Pharmaceutical carriers, diluents, and excipients are generally added to the peptide compositions or (peptide compositions kits) that are compatible with the active ingredients and acceptable for pharmaceutical use. Examples of such carriers include, but are not limited to, water, saline solutions, dextrose, and/or glycerol. Combinations of carriers can also be used. The vaccine compositions can further incorporate additional substances to stabilize pH and/or to function as adjuvants, wetting agents, and/or emulsifying agents, which can serve to improve the effectiveness of the vaccine.

The peptide compositions can include one or more adjuvants such but not limited to montanide ISA-51 (Seppic, Inc., Fairfield, N.J., United States of America); QS-21 STIMULON® brand adjuvant (Agenus Inc., Lexington, Mass., United States of America); ARLACEL® A brand mannide monooleate; oeleic acid; tetanus helper peptides (e.g., QYIKANSKFIGITEL (SEQ ID NO: 3972) or AQYIKANSKFIGITEL (SEQ ID NO: 3973); GM-CSF; cyclophosphamide; bacillus Calmette-Guerin (BCG); corynbacterium parvum; levamisole, azimezone; isoprinisone; dinitrochlorobenezene (DNCB); keyhole limpet hemocyanins (KLH) including Freunds adjuvant (complete and incomplete); mineral gels; aluminum hydroxide (Alum); lysolecithin; pluronic polyols; polyanions; peptides; oil emulsions; nucleic acids (e.g., dsRNA) dinitrophenol; diphtheria toxin (DT); toll-like receptor (TLR, e.g., TLR3, TLR4, TLR7, TLR8 or TLR9) agonists (e.g, endotoxins such as lipopolysaccharide (LPS); monophosphoryl lipid A (MPL); polyinosinic-polycytidylic acid (poly-ICLC/HILTONOL®; Oncovir, Inc., Wash., DC, United States of America); IMO-2055; glucopyranosyl lipid A (GLA); QS-21—a saponin extracted from the bark of the Quillaja saponaria tree, also known as the soap bark tree or Soapbark; resiquimod (TLR7/8 agonist), CDX-1401—a fusion protein consisting of a fully human monoclonal antibody with specificity for the dendritic cell receptor DEC-205 linked to the NY-ESO-1 tumor antigen; Juvaris' Cationic Lipid-DNA Complex; Vaxfectin; and combinations thereof.

Polyinosinic-Polycytidylic acid (Poly IC) is a double-stranded RNA (dsRNA) that acts as a TLR3 agonist. To increase half-life, it has been stabilized with polylysine and carboxymethylcellulose as poly-ICLC. It has been used to induce interferon in cancer patients, with intravenous doses up to 300 μg/kg. Like poly-IC, poly-ICLC is a TLR3 agonist. TLR3 is expressed in the early endosome of myeloid DC; thus poly ICLC preferentially activates myeloid dendritic cells, thus favoring a Th1 cytotoxic T-cell response. Poly ICLC activates natural killer (NK) cells, induces cytolytic potential, and induces IFN-gamma from myeloid DC.

In some embodiments, the adjuvant is provided at about or at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 micrograms per dose or per kg in each dose. In some embodiments, the adjuvant is provided at least or about 0.1, 0.2, 0.3, 0.40, 0.50, 0.60, 0.70, 0.80, 0.90, 0.100, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, 5.00, 5.10, 5.20, 5.30, 5.40, 5.50, 5.60, 5.70, 5.80, 5.90, 6.00, 6.10, 6.20, 6.30, 6.40, 6.50, 6.60, 6.70, 6.80, 6.90, 7.00, 7.10, 7.20, 7.30, 7.40, 7.50, 7.60, 7.70, 7.80, 7.90, 8.00, 8.10, 8.20, 8.30, 8.40, 8.50, 8.60, 8.70, 8.80, 8.90, 9.00, 9.10, 9.20, 9.30, 9.40, 9.50, 9.60, 9.70, 9.80, or 9.90 grams per dose or per kg in each dose. In some embodiments, the adjuvant is given at about or at least 10, 15, 20, 25, 50, 75, 100, 125, 150, 175, 150, 175, 200, 225, 250, 275, 300, 325, 350, 375, 400, 425, 450, 500, 525, 550, 575, 600, 625, 675, 700, 725, 750, 775, 800, 900, 1000, 1100, 1200, 1300, 1400, 1500, 1600, 1700, 1800, 1900, or 2000 endotoxin units (“EU”) per dose.

The peptide compositions of the presently disclosed subject matter can in some embodiments be provided with an administration of cyclophosphamide around the time, (e.g., about or at least 1, 2, 3, or 4 weeks or days before or after) the initial dose of a peptide composition. An exemplary dose of cyclophosphamide would in some embodiments be about or at least 100, 200, 300, 400, 500, 600, 700, 800, 900, or 1000 mg/m²/day over about or at least 1, 2, 3, 4, 5, 6, 7, 8, 9, or 10 days.

The compositions of the presently disclosed subject matter can in some embodiments comprise the presently disclosed peptides in the free form and/or in the form of a pharmaceutically acceptable salt.

As used herein, “a pharmaceutically acceptable salt” refers to a derivative of the disclosed peptides wherein the peptide is modified by making acid or base salts of the peptide. For example, acid salts are prepared from the free base (typically wherein the neutral form of the drug has a neutral —NH₂ group) involving reaction with a suitable acid. Suitable acids for preparing acid salts include both organic acids such as but not limited to acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids such as but not limited to hydrochloric acid, hydrobromic acid, sulfuric acid, nitric acid, phosphoric acid, and the like. Conversely, basic salts of acid moieties which can be present on a peptide are prepared using a pharmaceutically acceptable base such as sodium hydroxide, potassium hydroxide, ammonium hydroxide, calcium hydroxide, trimmethylamine or the like. By way of example and not limitation, the compositions can in some embodiments comprise the peptides as salts of acetic acid (acetates), ammonium, or hydrochloric acid (chlorides).

In some embodiments, a composition can include one or more sugars, sugar alcohols, amino acids such a glycine, arginine, glutaminic acid, and others as framework former. The sugars can be mono-, di- or trisaccharide. These sugars can be used alone, as well as in combination with sugar alcohols. Examples of sugars include glucose, mannose, galactose, fructose or sorbose as monosaccharides, sucrose, lactose, maltose or trehalose as disaccharides and raffinose as a trisaccharide. A sugar alcohol can be, for example, mannitose. In some embodiments, the composition comprises sucrose, lactose, maltose, trehalose, mannitol and/or sorbitol. In some embodiments, the composition comprises mannitol.

Furthermore, in some embodiments the presently disclosed compositions can include physiological well-tolerated excipients (see e.g., the Rowe et al., 2006), such as antioxidants like ascorbic acid or glutathione, preserving agents such as phenol, m-cresole, methyl- or propylparabene, chlorobutanol, thiomersal or benzalkoniumchloride, stabilizer, framework former such as sucrose, lactose, maltose, trehalose, mannitose, mannitol and/or sorbitol, mannitol and/or lactose and solubilizer such as polyethyleneglycols (PEG), i.e. PEG 3000, 3350, 4000, or 6000, or cyclodextrines, i.e. hydroxypropyle-β-cyclodextrine, sulfobutylethyl-β-cyclodextrine or γ-cyclodextrine, or dextranes or poloxaomers, i.e. poloxaomer 407, poloxamer 188, or TWEEN™20, TWEEN™80. In some embodiments, one or more well tolerated excipients can be included, selected from the group consisting of antioxidants, framework formers, and stabilizers.

In some embodiments, the pH for intravenous and intramuscular administration is selected from pH 2 to pH 12, while the pH for subcutaneous administration is selected from pH 2.7 to pH 9.0 as the rate of in vivo dilution is reduced resulting in more potential for irradiation at the injection site. (Strickley, 2004).

It is understood that a suitable dosage of a peptide composition vaccine immunogen will depend upon the age, sex, health, and weight of the recipient, the kind of concurrent treatment, if any, the frequency of treatment, and the nature of the effect desired. However, a desired dosage can be tailored to the individual subject, as determined by the researcher or clinician. The total dose employed for any given treatment can typically be determined with respect to a standard reference dose based on the experience of the researcher or clinician, such dose being administered either in a single treatment or in a series of doses, the success of which can depend on the production of a desired immunological result (i.e., successful production of a T helper cell and/or CTL-mediated response to the peptide immunogen composition, which response gives rise to the prevention and/or treatment desired). Thus, in some embodiments the overall administration schedule can be considered in determining the success of a course of treatment and not whether a single dose, given in isolation, would or would not produce the desired immunologically therapeutic result or effect. As such, a therapeutically effective amount (i.e., that producing the desired T helper cell and/or CTL-mediated response) can in some embodiments depend on the antigenic composition of the vaccine used, the nature of the disease condition, the severity of the disease condition, the extent of any need to prevent such a condition where it has not already been detected, the manner of administration dictated by the situation requiring such administration, the weight and state of health of the individual receiving such administration, and/or the sound judgment of the clinician or researcher. Needless to say, the efficacy of administering additional doses and of increasing or decreasing the interval can be re-evaluated on a continuing basis, in view of the recipient's immunocompetence (for example, the level of T helper cell and/or CTL activity with respect to tumor-associated or tumor-specific antigens).

The concentration of the T helper or CTL stimulatory peptides of the presently disclosed subject matter in pharmaceutical formulations are subject to wide variation, including anywhere from less than 0.01% by weight to as much as 50% or more. Factors such as volume and viscosity of the resulting composition can also be considered. The solvents, or diluents, used for such compositions can include one or more of water, phosphate buffered saline (PBS), saline itself, and/or other possible carriers and/or excipients. The immunogens of the presently disclosed subject matter can in some embodiments also be contained in artificially created structures such as liposomes, which structures can in some embodiments contain additional molecules, such as proteins or polysaccharides, inserted in the outer membranes of the structures and having the effect of targeting the liposomes to particular areas of the body, or to particular cells within a given organ or tissue. Such targeting molecules can in some embodiments be some type of immunoglobulin. Antibodies can work particularly well for targeting the liposomes to tumor cells.

Single i.d., i.m., s.c., i.p., and/or i.v. doses of e.g., about 1 to 50 μg to 100 μg to 500 μg, 1 to 1000 μg or about 1 to 50 mg, 1 to 100 mg, 1 to 500 mg, or 1 to 1000 mg of a peptide composition of the presently disclosed subject matter can in some embodiments be given and in some embodiments can depend from the respective compositions of peptides with respect to total amount for all peptides in the composition or alternatively for each individual peptide in the composition. A single dose of a peptide vaccine composition of the presently disclosed subject matter can in some embodiments have a peptide amount (e.g., total amount for all peptides in the composition or alternatively for each individual peptide in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 Alternatively, a single dose of a peptide composition of the presently disclosed subject matter can in some embodiments have a total peptide amount (e.g., total amount for all peptides in the composition or alternatively for each individual peptide in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 mg. In some embodiments, the peptides of a composition of the presently disclosed subject matter are present in equal amounts of about 100 micrograms per dose in combination with an adjuvant peptide present in an amount of about 200 micrograms per dose.

In a single dose of the peptide composition of the presently disclosed subject matter, the amount of each peptide in the composition is in some embodiments equal or is in some embodiments substantially equal. Alternatively, the ratio of the peptides present in the least amount relative to the peptide present in the greatest amount is in some embodiments about or at least 1:1.25, 1:1.5, 1:1.75, 1:2.0, 1:2.25, 1:2.5, 1:2.75, 1:3, 1:4, 1:5, 1:6, 1:7, 1:8, 1:9, 1:10, 1:20, 1:30; 1:40, 1:50, 1:100, 1:200, 1:500, 1:1000, 1:5000;

1:10,000; or 1:100,000. Alternatively, the ratio of the peptides present in the least amount relative to the peptide present in the greatest amount is in some embodiments about or at least 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15 to 20; 20 to 25; 1 to 40; 1 to 30; 1 to 20; 1 to 15; 10 to 40; 10 to 30; 10 to 20; 10 to 15; 20 to 40; 20 to 30; or 20 to 25; 1 to 100; 25 to 100; 50 to 100; 75 to 100; 25 to 75, 25 to 50, or 50 to 75; 25 to 40; 25 to 50; 30 to 50; 30 to 40; or 30 to 75.

Single dosages can in some embodiments be given to a patient about or at least 1, 2, 3, 4, or 5 times per day. Single dosages can in some embodiments be given to a patient about or at least 1, 2, 3, 4, 5, 6, 7, 8, 9, 10, 11, 12, 13, 14, 15, 16, 18, 19, 20, 21, 22, 23, 24, 36, 48, 60, or 72 hours subsequent to a previous dose.

Single dosages can in some embodiments be given to a patient about or at least 1, 2, 3, 4, 5, 6, or 7 times per week or every other, third, fourth, or fifth day. Single doses can in some embodiments also be given every week, every other week, or only during 1, 2, or 3 weeks per month. A course of treatment can in some embodiments last about or at least 1, 2, 3, 4, 5, 6, 7, 8, 9, 10, 11, or 12 months.

In some embodiments, single dosages of the compositions of the presently disclosed subject matter are provided to a patient in at least two phases, e.g., during an initial phase and then a subsequent phase. An initial phase can in some embodiments be about or at least 1, 2, 3, 4, 5, or 6 weeks in length. The subsequent phase can in some embodiments last at least or about 1, 2, 3, 4, 5, 6, 7, or 8 times as long as the initial phase. The initial phase can in some embodiments be separated from the subsequent phase by about or at least 1, 2, 3, 4, 5, 6, 7, 8, 9, 10, 11, or 12 weeks or months.

The peptide composition dosage during the subsequent phase can in some embodiments be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times greater than during the initial phase. The peptide composition dosage during the subsequent phase can in some embodiments be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times lower than during the initial phase.

In some embodiments, the initial phase is about three weeks and the second phase is about 9 weeks. In some embodiments, the peptide compositions would be administered to the patient on or about days 1, 8, 15, 36, 57, and 78.

In some embodiments, the presently disclosed subject matter provides a kit. In some embodiments the kit comprises (a) a container that contains at least one peptide composition as described herein in solution or in lyophilized form; (b) optionally, a second container containing a diluent or reconstituting solution for the lyophilized formulation; and (c) also optionally, instructions for (i) use of the solution; and/or (ii) reconstitution and/or use of the lyophilized formulation. The kit can in some embodiments further comprise one or more of (iii) a buffer, (iv) a diluent, (v) a filter, (vi) a needle, and/or (v) a syringe. In some embodiments, the container is selected from the group consisting of a bottle, a vial, a syringe, a test tube, and a multi-use container. In some embodiments, the peptide composition is lyophilized.

The kits can in some embodiments contain exactly, about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, or more peptide-containing compositions. Each composition in the kit can in some embodiments be administered at the same time or at different times to a subject.

In some embodiments, the kits can comprise a lyophilized formulation of the presently disclosed compositions and/or vaccines in a suitable container and instructions for its reconstitution and/or use. Suitable containers include, for example, bottles, vials (e.g. dual chamber vials), syringes (such as dual chamber syringes), and test tubes. The container can in some embodiments be formed from a variety of materials such as glass or plastic. In some embodiments, the kit and/or container include instructions on or associated with the container that indicate directions for reconstitution and/or use. For example, the label can in some embodiments indicate that the lyophilized formulation is to be reconstituted to peptide concentrations as described above. The label can in some embodiments further indicate that the formulation is useful or intended for subcutaneous administration. Lyophilized and liquid formulations are in some embodiments stored at −20° C. to −80° C.

The container holding the peptide composition(s) can in some embodiments be a multi-use vial, which allows for repeat administrations (e.g., from 2-6 administrations) of the reconstituted formulation. The kit can in some embodiments further comprise a second container comprising a suitable diluent such as, but not limited to a sodium bicarbonate solution.

In some embodiments, upon mixing of the diluent and the lyophilized formulation, the final peptide concentration in the reconstituted formulation is at least or about 0.15, 0.20, 0.25, 0.5, 0.75, 1.0, 1.25, 1.5, 1.75, 2.0, 2.25, 2.5, 2.75, 3.0, 3.25, 3.50, 3.75, 4.0, 4.25, 4.5, 4.75, 5.0, 6.0, 7.0, 8.0, 9.0, or 10 mg/mL/peptide. In some embodiments, upon mixing of the diluent and the lyophilized formulation, the final peptide concentration in the reconstituted formulation is at least or about 0.15, 0.20, 0.25, 0.5, 0.75, 1.0, 1.25, 1.5, 1.75, 2.0, 2.25, 2.5, 2.75, 3.0, 3.25, 3.50, 3.75, 4.0, 4.25, 4.5, 4.75, 5.0, 6.0, 7.0, 8.0, 9.0 or 10 μg/mL/peptide.

The kit can in some embodiments further comprise other materials desirable from a commercial and user standpoint, including but not limited to other buffers, diluents, filters, needles, syringes, and/or package inserts with instructions for use.

The kits can in some embodiments have a single container that comprises the formulation of the peptide compositions with or without other components (e.g., other compounds or compositions of these other compounds) or can in some embodiments have a distinct container for each component.

Additionally, the kits can in some embodiments comprise a formulation of the presently disclosed peptide compositions and/or vaccines packaged for use in combination with the co-administration of a second compound such as but not limited to adjuvants (e.g. imiquimod), a chemotherapeutic agent, a natural product, a hormone or antagonist, an anti-angiogenesis agent or inhibitor, an apoptosis-inducing agent, or a chelator or a composition thereof. The components of the kit can in some embodiments be pre-complexed or each component can in some embodiments be in a separate distinct container prior to administration to a patient. The components of the kit can in some embodiments be provided in one or more liquid solutions. In some embodiments, the liquid solution is an aqueous solution. In some embodiments, the liquid solution is a sterile aqueous solution. The components of the kit can in some embodiments also be provided as solids, which in some embodiments are converted into liquids by addition of suitable solvents, which can in some embodiments be provided in another distinct container.

The container of a therapeutic kit can in some embodiments be a vial, a test tube, a flask, a bottle, a syringe, or any other article suitable to enclose a solid or liquid. In some embodiments, when there is more than one component, the kit can contain a second vial and/or other container, which allows for separate dosing. The kit can in some embodiments also contain another container for a pharmaceutically acceptable liquid. In some embodiments, a therapeutic kit contains an apparatus (e.g., one or more needles, syringes, eye droppers, pipette, etc.) that facilitates administration of the agents of the disclosure that are components of the present kit.

When administered to a patient, the vaccine compositions of the presently disclosed subject matter are envisioned to have certain physiological effects, including but not limited to the induction of a T cell mediated immune response. In some embodiments, the vaccine compositions of the presently disclosed subject matter induce and anti-tumor immune response and/or an anti-cancer immune response. In some embodiments, the vaccine compositions of the presently disclosed subject matter are envisioned to have an anti-microbial immune response, which in some embodiments can be an anti-bacterial immune response, an anti-viral immune response, or a combination thereof.

Immunohistochemistry, Immunofluorescence, Western Blots, and Flow Cytometry

Validation and testing of antibodies for characterization of cellular and molecular features of lymphoid neogenesis has been performed. Commercially available antibodies for use in immunohistochemistry (IHC), immunofluorescence (IF), flow cytometry (FC), and western blot (WB) can in some embodiments be employed. In some embodiments, such techniques can be employed to analyze patient samples, e.g., formalin-fixed, paraffin-embedded tissue samples, for CD1a, S100, CD83, DC-LAMP, CD3, CD4, CD8, CD20, CD45, CD79a, PNAd, TNFalpha, LIGHT, CCL19, CCL21, CXCL12, TLR4, TLR7, FoxP3, PD1 and Ki67 expression. In some embodiments, flow cytometry is used to determine CD3, CD4, CD8, CD13, CD14, CD16, CD19, CD45RA, CD45RO, CD56, CD62L, CD27, CD28, CCR7, FoxP3 (intracellular), and MHC-peptide tetramers for I MHC associated (phospho)-peptides. In some embodiments, positive control tissue selected from among normal human peripheral blood lymphocytes (PBL), PBL activated with CD3/CD28 beads (activated PBL), human lymph node tissue from non-HCC patients (LN), and inflamed human tissue from a surgical specimen of Crohn's disease (Crohn's) can be employed.

ELISpot Assay

In some embodiments, vaccination site infiltrating lymphocytes and lymphocytes from the sentinel immunized nod (SIN) and vaccine site can be evaluated by ELISpot. ELISpot permits the direct counting of T-cells reacting to antigen by production of INFγ. Peripheral blood lymphocytes can be evaluated by ELISpot assay for the number of peptide-reactive T-cells. Vaccine site infiltrating lymphocytes and SIN lymphocytes can be compared to those in peripheral blood. It is envisioned that positive results of the ELISpot assay correlate with increased patient progression free survival. Progression free survival is in some embodiments defined as the time from start of treatment until death from any cause or date of last follow up.

Tetramer Assay

Peripheral blood lymphocytes and lymphocytes from the SIN and vaccine site can be evaluated by flow cytometry after incubation with MHC-peptide tetramers for the number of peptide-reactive T-cells.

Proliferation Assay/Cytokine Analysis

Peripheral blood mononuclear cells (PBMC), vaccine-site inflammatory cells, and lymphocytes from the SIN from patients can in some embodiments be evaluated for CD4 T cell reactivity to, e.g., tetanus helper peptide mixture, using a ³H-thymidine uptake assay. Additionally, Th1 (IL-2, IFN-gamma, TNFa), Th2 (IL-4, IL-5, IL-10), Th17 (IL-17, and IL23), and T-reg (TGF-beta) cytokines in media from 48 hours in that proliferation assay can be employed to determine if the microenvironment supports generation of Th1, Th2, Th17, and/or T-reg responses. In some embodiments, two peptides are used as negative controls: a tetanus peptide and the Pan DR T helper epitopes (PADRE) peptide (AK(X)VAAWTLKAA; SEQ ID NO: 3974).

Evaluation of Tumors

In some embodiments tumor tissue collected prior to treatment or at the time of progression can be evaluated by routine histology and immunohistochemistry. Alternatively or in addition, in vitro evaluations of tumor tissue and tumor infiltrating lymphocytes can be completed.

Studies of Homing Receptor Expression

Patient samples can in some embodiments be studied for T cell homing receptors induced by vaccination the compositions of the presently disclosed subject matter. These include, but are not limited to, integrins (including alphaE-beta7, alpha1-beta1, alpha4-beta1), chemokine receptors (including CXCR3), and selectin ligands (including CLA, PSL) on lymphocytes, and their ligands in the vaccine sites and SIN. These can be assayed by immunohistochemistry, flow cytometry or other techniques.

Studies of Gene and Protein Expression

Differences in gene expression and/or for differences in panels of proteins can in some embodiments be assayed by high-throughput screening assays (e.g. nucleic acid chips, protein arrays, etc.) in the vaccine sites and sentinel immunized nodes.

### VII. Antibodies Including Antibody-Like Molecules

In some embodiments, the present disclosure provides antibodies and antibody-like molecules (e.g. T cell receptors) that specifically bind to the peptides (e.g., phosphopeptides) disclosed herein, or to complexes of an MHC molecule (e.g., a class I MHC fmolecule) and the peptides disclosed herein. In some embodiments, the antibodies and antibody-like molecules (e.g. T cell receptors) specifically bind to complexes of phosphopeptides and corresponding MHC alleles as set forth in Tables 3-7.

Antibodies and antibody-like molecules (e.g. T cell receptors) specific for peptides or peptide/MHC complexes are, for example, useful, inter alia, for analyzing tissue to determine the pathological nature of tumor margins and/or can be employed in some embodiments as therapeutics. Alternatively, such molecules can in some embodiments be employed as therapeutics targeting cells, e.g., tumor cells, which display peptides on their surface. In some embodiments, the antibodies and antibody-like molecules bind the peptides or peptide-MHC complex specifically and do not substantially cross react with non-phosphorylated native peptides.

As used herein, “antibody” and “antibody peptide(s)” refer to intact antibodies, antibody-like molecules, and binding fragments thereof that compete with intact antibodies for specific binding. Binding fragments are in some embodiments produced by recombinant DNA techniques or in some embodiments by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody in some embodiments substantially inhibits adhesion of a receptor to a counterreceptor when an excess of antibody reduces the quantity of receptor bound to counterreceptor by at least about 20%, 40%, 60%, 80%, 85%, 90%, 91%, 92%, 93%, 94%, 95%, 96%, 97%, 98%, 99%, or greater than 99% as measured, for example, in an in vitro competitive binding assay.

The term “MHC” as used herein refers to the Major Histocompability Complex, which is defined as a set of gene loci specifying major histocompatibility antigens. The term “HLA” as used herein refers to Human Leukocyte Antigens, which are defined as the histocompatibility antigens found in humans. As used herein, “HLA” is the human form of “MHC”.

The terms “MHC light chain” and “MHC heavy chain” as used herein refer to portions of MHC molecules. Structurally, class I molecules are heterodimers comprised of two non-covalently bound polypeptide chains, a larger “heavy” chain (α) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3 domain of the molecule contains the recognition site for the CD8 protein on the CTL; this interaction serves to stabilize the contact between the T cell and the APC.

The invariant light chain (12 kDa), encoded outside the MEW on chromosome 15, consists of a single, extracellular polypeptide. The terms “MHC light chain”, “β-2-microglobulin”, and “β2m” are used interchangeably herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody or antibody like molecule is said to “specifically” bind an antigen when the dissociation constant is in some embodiments less than 1 μM, in some embodiments less than 100 nM, and in some embodiments less than 10 nM.

The term “antibody” is used in the broadest sense, and specifically covers monoclonal antibodies (including full length monoclonal antibodies), polyclonal antibodies, multispecific antibodies (e.g., bi specific antibodies), and antibody fragments (e.g., Fab, F(ab′)2 and Fv), as well as “antibody-like molecules” so long as they exhibit the desired biological activity. Antibodies (Abs) and immunoglobulins (Igs) are glycoproteins having the same structural characteristics. The term is also meant to encompass “antibody like molecules” and other members of the immunoglobulin superfamily, e.g., T-cell receptors, MEW molecules, containing e.g., an antigen-binding regions and/or variable regions, e.g., complementary determining regions (CDRs) which specifically bind the peptides disclosed herein.

In some embodiments, antibodies and antibody-like molecules bind to the peptides of the presently disclosed subject matter but do not substantially and/or specifically cross react with the same peptide in a modified form. See e.g., U.S. Patent Application Publication No. 2009/0226474, which is incorporated by reference.

The presently disclosed subject matter also includes antibodies that recognize peptides associated with a tumorigenic or disease state, wherein the peptides are displayed in the context of HLA molecules. These antibodies typically mimic the specificity of a T cell receptor (TCR) but can in some embodiments have higher binding affinity such that the molecules can be employed as therapeutic, diagnostic, and/or research reagents. Methods of producing a T-cell receptor mimic of the presently disclosed subject matter include identifying a peptide of interest (e.g., a phosphopeptide), wherein the peptide of interest comprises an amino acid sequence as set forth in any of SEQ ID NOs: 1-3921 and 3975-4000 (e.g., a phosphopeptide as set forth in Tables 3-7 herein). Then, an immunogen comprising at least one peptide/MHC complex is formed. An effective amount of the immunogen is then administered to a host for eliciting an immune response, and serum collected from the host is assayed to determine if desired antibodies that recognize a three-dimensional presentation of the peptide in the binding groove of the MHC molecule are being produced. The desired antibodies can differentiate the peptide/MHC complex from the MHC molecule alone, the peptide alone, and a complex of MHC and irrelevant peptide. Finally, in some embodiments the desired antibodies are isolated.

The term “antibody” also encompasses soluble T cell receptors (TCR) which are stable at low concentrations and which can recognize MHC-peptide complexes. See e.g., U.S. Patent Application Publication No. 2002/0119149, which is incorporated by reference. Such soluble TCRs might for example be conjugated to immunostimulatory peptides and/or proteins or moieties, such as CD3 agonists (anti-CD3 antibody), for example. The CD3 antigen is present on mature human T cells, thymocytes, and a subset of natural killer cells. It is associated with the TCR and is responsible for the signal transduction of the TCR.

Antibodies specific for the human CD3 antigen are well-known. One such antibody is the murine monoclonal antibody OKT3 which was the first monoclonal antibody approved by the FDA. OKT3 is reported to be a potent T cell mitogen (see e.g., Van Wauve, 1980; U.S. Pat. No. 4,361,539) and a potent T cell killer (Wong, 1990. Other antibodies specific for the CD3 antigen have also been reported (see e.g., PCT International Patent Application Publication No. WO 2004/0106380; U.S. Patent Application Publication No. 2004/0202657; U.S. Pat. Nos. 6,750,325; 6,706,265; GB 2249310A; Clark et al., 1989; U.S. Pat. No. 5,968,509; and U.S. Patent Application Publication No. 2009/0117102). ImmTACs (Immunocore Limited, Milton Park, Abington, Oxon, United Kingdom) are innovative bifunctional proteins that combine high-affinity monoclonal T cell receptor (mTCR) targeting technology with a clinically-validated, highly potent therapeutic mechanism of action (Anti-CD3 scFv).

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. The number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Chothia et al., 1985; Novotny & Haber, 1985).

An “isolated” antibody is one which has been separated, identified, and/or recovered from a component of the environment in which it was produced. Contaminant components of its production environment are materials which would interfere with diagnostic or therapeutic uses for the antibody, and can include enzymes, hormones, and other proteinaceous or nonproteinaceous solutes. In some embodiments, the antibody is purified as measurable by at least one of the following three different methods: 1) to in some embodiments greater than 50% by weight of antibody as determined by the Lowry method, such as but not limited to in some embodiments greater than 75% by weight, in some embodiments greater than 85% by weight, in some embodiments greater than 95% by weight, in some embodiments greater than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, in some embodiments, silver stain. Isolated antibodies include the antibody in situ within recombinant cells since at least one component of the antibody's natural environment is not present. In some embodiments, however, isolated antibodies are prepared by a method that includes at least one purification step.

The terms “antibody mutant”, “antibody variant”, and “antibody derivative” refer to an amino acid sequence variant of an antibody wherein one or more of the amino acid residues of a reference antibody has been modified (e.g., substituted, deleted, chemically modified, etc.). Such mutants necessarily have less than 100% sequence identity or similarity with the amino acid sequence of either the heavy or light chain variable domain of the reference antibody. The resultant sequence identity or similarity between the modified antibody and the reference antibody is thus in some embodiments at least 80%, in some embodiments at least 85%, in some embodiments at least 90%, in some embodiments at least 95%, in some embodiments at least 97%, and in some embodiments at least 99%.

The term “variable” in the context of variable domain of antibodies, refers to the fact that certain portions of the variable domains differ extensively in sequence among antibodies and are used in the binding and specificity of each particular antibody for its particular antigen(s). However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (Kabat et al., 1987); and (2) an approach based on crystallographic studies of antigen-antibody complexes (Chothia et al., 1989). The more highly conserved portions of variable domains are called the framework (FR) regions. The variable domains of native heavy and light chains each comprise four FR regions, largely adopting a β-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al., 1987). The constant domains are not involved directly in binding an antibody to an antigen, but exhibit various effector function, such as participation of the antibody in antibody-dependent cellular toxicity.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, “functional fragment” with respect to antibodies, refers to Fv, F(ab) and F(ab′)₂ fragments.

An “Fv” fragment is the minimum antibody fragment which contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa and lambda, based on the amino sequences of their constant domain.

Depending on the amino acid sequences of the constant domain of their heavy chains, immunoglobulins can be assigned to different classes. There are at least five (5) major classes of immunoglobulins: IgA, IgD, IgE, IgG and IgM, and several of these can be further divided into subclasses (isotypes), e.g., IgG₁, IgG₂, IgG₃, and IgG₄; IgA₁ and IgA₂. The heavy chains constant domains that correspond to the different classes of immunoglobulins are called alpha (α), delta (Δ), epsilon (ε), gamma (γ), and mu (μ), respectively. The subunit structures and three-dimensional configurations of different classes of immunoglobulins are well-known.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, monoclonal antibodies can be advantageous in that they can be synthesized in hybridoma culture, uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter can in some embodiments be made by the hybridoma method first described by Kohler & Milstein, 1975, or can in some embodiments be made by recombinant methods, e.g., as described in U.S. Pat. No. 4,816,567. The monoclonal antibodies for use with the presently disclosed subject matter can in some embodiments also be isolated from phage antibody libraries using the techniques described in Clackson et al., 1991 or in Marks et al., 1991.

Utilization of the monoclonal antibodies of the presently disclosed subject matter can in some embodiments require administration of such or similar monoclonal antibody to a subject, such as a human. However, when the monoclonal antibodies are produced in a non-human animal, such as a rodent, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed subject matter can be “humanized”: that is, the antibodies can be engineered such that antigenic portions thereof are removed and like portions of a human antibody are substituted therefor, while the antibodies' affinity for specific peptide/MHC complexes is retained. This engineering can in some embodiments only involve a few amino acids, or can in some embodiments include entire framework regions of the antibody, leaving only the complementarity determining regions of the antibody intact. Several methods for humanizing antibodies are known in the art and are disclosed, for example, in U.S. Pat. Nos. 4,816,567; 5,712,120; 5,861,155; 5,869,619; 6,054,927; and 6,180,370; the entire content of each of which is hereby expressly incorporated herein by reference in its entirety.

Humanized forms of antibodies are chimeric immunoglobulins, immunoglobulin chains, or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. In some embodiments, humanization can be performed following the method of Winter and co-workers (see e.g., Jones et al., 1986; Riechmann et al., 1988; Verhoeyen et al., 1988) by substituting rodent CDRs or CDR sequences for the corresponding sequences of a human antibody. See also U.S. Pat. No. 5,225,539. In some embodiments, Fᵥ framework residues of a human immunoglobulin are replaced by corresponding non-human residues.

Humanized antibodies can also comprise residues which are found neither in the recipient antibody nor in the imported CDR or framework sequences. In general, a humanized antibody comprises substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally can in some embodiments also comprise at least a portion of an immunoglobulin constant region (Fc), typically that of a human immunoglobulin. See e.g., Jones et al., 1986; Riechmann et al., 1988; Presta, 1992.

Many articles relating to the generation or use of humanized antibodies teach useful examples of protocols that can be utilized with the presently disclosed subject matter, such as but not limited to Shinkura et al., 1998; Yenari et al., 1998; Richards et al., 1999; Morales et al., 2000; Mihara et al., 2001; Sandborn et al., 2001; and Yenari et al., 2001, all of which are expressly incorporated in their entireties by reference. For example, a treatment protocol that can be utilized in such a method includes a single dose, generally administered intravenously, of 10-20 mg of humanized mAb per kg (Sandborn et al., 2001). In some embodiments, alternative dosing patterns can be appropriate, such as but not limited to the use of three infusions, administered once every two weeks, of 800 to 1600 mg or even higher amounts of humanized mAb (Richards et al., 1999.). However, it is to be understood that the presently disclosed subject matter is not limited to the treatment protocols described above, and other treatment protocols that are known to a person of ordinary skill in the art can be utilized in the methods of the presently disclosed subject matter.

The presently disclosed and claimed subject matter further includes in some embodiments fully human monoclonal antibodies against specific peptide/MHC complexes. Fully human antibodies essentially relate to antibody molecules in which the entire sequence of both the light chain and the heavy chain, including the CDRs, arise from human genes. Such antibodies are referred to herein as “human antibodies” or “fully human antibodies”. Human monoclonal antibodies can be prepared by the trioma technique; the human B-cell hybridoma technique (see Kozbor et al., 1983), and the EBV hybridoma technique to produce human monoclonal antibodies (see Cole et al., 1985). Human monoclonal antibodies can in some embodiments be utilized in the practice of the presently disclosed subject matter and can in some embodiments be produced by using human hybridomas (see Cote et al., 1983)) or by transforming human B-cells with Epstein Barr Virus in vitro (see Cole et al., 1985).

In addition, human antibodies can also be produced using additional techniques, including but not limited to phage display libraries (Hoogenboom et al., 1991; Marks et al., 1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016; and in Marks et al., 1992; Lonberg et al., 1994; Lonberg & Huszar, 1995; Fishwild et al., 1996; Neuberger, 1996.

Human antibodies can in some embodiments additionally be produced using transgenic nonhuman animals which are modified so as to produce fully human antibodies rather than the animal's endogenous antibodies in response to challenge by an antigen. See PCT International Patent Application Publication No. WO 1994/02602). Typically, the endogenous genes encoding the heavy and light immunoglobulin chains in the non-human host are incapacitated, and active loci encoding human heavy and light chain immunoglobulins are inserted into the host's genome. The human genes are incorporated, for example, using yeast artificial chromosomes containing the requisite human DNA segments. An animal that provides all the desired modifications is then obtained as progeny by crossbreeding intermediate transgenic animals containing fewer than the full complement of the modifications.

A non-limiting example of such a nonhuman animal is a mouse, and is termed the XENOMOUSE™ as disclosed in PCT International Patent Application Publication Nos. WO 1996/33735 and WO 1996/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a non-human host, exemplified as a mouse, lacking expression of an endogenous immunoglobulin heavy chain is disclosed in U.S. Pat. No. 5,939,598, incorporated herein by reference). It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

An exemplary method for producing an antibody of interest, such as a human antibody, is disclosed in U.S. Pat. No. 5,916,771 incorporated herein by reference). It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

The antigen peptides are known to be expressed on a variety of cancer cell types. Thus, antibodies and antibody-like molecules can be used where appropriate, in treating, diagnosing, vaccinating, preventing, retarding, and/or attenuating HCC, melanoma, ovarian cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer.

The antigen peptides are known to be expressed on a variety of microbial infected cells.

Antibodies generated with specificity for the antigen peptides can be used to detect the corresponding peptides in biological samples. The biological sample could come from an individual who is suspected of having cancer and thus detection would serve to diagnose the cancer. Alternatively, the biological sample can in some embodiments come from an individual known to have cancer, and detection of the antigen peptides would serve as an indicator of disease prognosis, cancer characterization, or treatment efficacy. Appropriate immunoassays are well-known in the art and include, but are not limited to, immunohistochemistry, flow cytometry, radioimmunoassay, western blotting, and ELISA. Biological samples suitable for such testing include, but are not limited to, cells, tissue biopsy specimens, whole blood, plasma, serum, sputum, cerebrospinal fluid, pleural fluid, and urine. Antigens recognized by T cells, whether helper T lymphocytes or CTL, are not recognized as intact proteins, but rather as small peptides that associate with class I or class II MHC proteins on the surface of cells. During the course of a naturally occurring immune response antigens that are recognized in association with class II MHC molecules on antigen presenting cells are acquired from outside the cell, internalized, and processed into small peptides that associate with the class II MHC molecules. Conversely, the antigens that give rise to proteins that are recognized in association with class I MHC molecules are generally proteins made within the cells, and these antigens are processed and associate with class I MHC molecules. It is now well-known that the peptides that associate with a given class I or class II MHC molecule are characterized as having a common binding motif, and the binding motifs for a large number of different class I and II MHC molecules have been determined. It is also well-known that synthetic peptides can be made which correspond to the sequence of a given antigen and which contain the binding motif for a given class I or II MHC molecule. These peptides can then be added to appropriate antigen presenting cells, and the antigen presenting cells can be used to stimulate a T helper cell or CTL response either in vitro or in vivo. The binding motifs, methods for synthesizing the peptides, and methods for stimulating a T helper cell or CTL response are all well-known and readily available.

As used herein, the terms “T cell receptor” and “TCR” are used interchangeably and refer to full length heterodimeric αβ or γδ TCRs, antigen-binding fragments of TCRs, or molecules comprising TCR CDRs or variable regions. Examples of TCRs include, but are not limited to, full-length TCRs, antigen-binding fragments of TCRs, soluble TCRs lacking transmembrane and cytoplasmic regions, single-chain TCRs containing variable regions of TCRs attached by a flexible linker, TCR chains linked by an engineered disulfide bond, monospecific TCRs, multi-specific TCRs (including bispecific TCRs), TCR fusions, human TCRs, humanized TCRs, chimeric TCRs, recombinantly produced TCRs, and synthetic TCRs. The term encompasses wild-type TCRs and genetically engineered TCRs (e.g., a chimeric TCR comprising a chimeric TCR chain which includes a first portion from a TCR of a first species and a second portion from a TCR of a second species).

As used herein, the term “TCR variable region” is understood to encompass amino acids of a given TCR which are not included within the non-variable region as encoded by the TRAC gene for TCR α chains and either the TRBC1 or TRBC2 genes for TCR β chains. In some embodiments, a TCR variable region encompasses all amino acids of a given TCR which are encoded by a TRAV gene or a TRAJ gene for a TCR α chain or a TRBV gene, a TRBD gene, or a TRBJ gene for a TCR β chain (see e.g., LeFranc & LeFranc, 2001, which is incorporated by reference herein in its entirety).

As used herein, the term “constant region” with respect to a TCR refers to the extracellular portion of a TCR that is encoded by the TRAC gene for TCR α chains and either the TRBC1 or TRBC2 genes for TCR β chains. The term constant region does not include a TCR variable region encoded by a TRAV gene or a TRAJ gene for a TCR α chain or a TRBV gene, a TRBD gene, or a TRBJ gene for a TCR β chain (see e.g., LeFranc & LeFranc, 2001, which is incorporated by reference herein in its entirety).

Kits can in some embodiments be composed for help in diagnosis, monitoring, and/or prognosis. The kits are to facilitate the detecting and/or measuring of cancer-specific peptides or proteins. Such kits can in some embodiments contain in a single or divided container, a molecule comprising an antigen-binding region. Such molecules can in some embodiments be antibodies and/or antibody-like molecules. Additional components that can be included in the kit include, for example, solid supports, detection reagents, secondary antibodies, instructions for practicing, vessels for running assays, gels, control samples, and the like. The antibody and/or antibody-like molecules can in some embodiments be directly or indirectly labeled, as an option.

Alternatively or in addition, the antibody or antibody-like molecules specific for peptides and/or peptide/MHC complexes can in some embodiments be conjugated to therapeutic agents. Exemplary therapeutic agents include anti-cancer agents, anti-tumor agents, antimicrobial agents, antivirals, and therapeutic agents for use in treating neurological diseases including but not limited to Alzheimer's disease.

Alkylating Agents: Alkylating agents are drugs that directly interact with genomic DNA to prevent cells from proliferating. This category of chemotherapeutic drugs represents agents that affect all phases of the cell cycle, that is, they are not phase-specific. An alkylating agent can in some embodiments include, but is not limited to, a nitrogen mustard, an ethylenimene, a methylmelamine, an alkyl sulfonate, a nitrosourea or a triazines. They include but are not limited to busulfan, chlorambucil, cisplatin, cyclophosphamide (cytoxan), dacarbazine, ifosfamide, mechlorethamine (mustargen), and melphalan.

Antimetabolites: Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to 5-fluorouracil (5-FU), cytarabine (Ara-C), fludarabine, gemcitabine, and methotrexate.

Natural Products: Natural products generally refer to compounds originally isolated from a natural source, and identified as having a pharmacological activity. Such compounds, as well as analogs and derivatives thereof, can in some embodiments be isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include, but are not limited to, compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

Antibiotics: Certain antibiotics have both antimicrobial and cytotoxic activity. These drugs can also interfere with DNA by chemically inhibiting enzymes and mitosis or altering cellular membranes. These agents are typically not phase-specific so they work in all phases of the cell cycle. Examples of cytotoxic antibiotics include but are not limited to bleomycin, dactinomycin, daunorubicin, doxorubicin (Adriamycin), plicamycin (mithramycin), and idarubicin.

Miscellaneous Agents: Miscellaneous cytotoxic agents that do not fall into the previous categories include but are not limited to platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are not limiting and it is contemplated that any known cytotoxic, cytostatic, and/or cytocidal agent can be conjugated or otherwise attached to targeting peptides and administered to a targeted organ, tissue, and/or cell type within the scope of the presently disclosed subject matter.

Chemotherapeutic (cytotoxic) agents include but are not limited to 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raioxifene, tamoxifen, taxol, temazolomide (an aqueous form of DTIC), transplatinum, vinblastine and methotrexate, vincristine, or any analog or derivative variant of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

The peptides identified and tested thus far in peptide-based vaccine approaches have generally fallen into one of three categories: 1) mutated on individual tumors, and thus not displayed on a broad cross section of tumors from different patients; 2) derived from unmutated tissue-specific proteins, and thus compromised by mechanisms of self-tolerance; and 3) expressed in subsets of cancer cells and normal testes.

Antigens linked to transformation or oncogenic processes are of primary interest for immunotherapeutic development based on the hypothesis that tumor escape through mutation of these proteins can be more difficult without compromising tumor growth or metastatic potential.

The peptides of the presently disclosed subject matter are unique in that the identified peptides are modified by intracellular modification. This modification is of particular relevance because it is associated with a variety of cellular control processes, some of which are dysregulated in cancer cells. For example, the source proteins for class I MHC-associated phosphopeptides are often known phosphoproteins, supporting the idea that the phosphopeptides are processed from folded proteins participating in signaling pathways.

Although not wishing to be bound by any particular theory, it is envisioned that the peptides of the presently disclosed subject matter are unexpectedly superior to known tumor-associated antigen-derived peptides for use in immunotherapy because: 1) they only displayed on the surface of cells in which intracellular phosphorylation is dysregulated, i.e., cancer cells, and not normal thymus cells, and thus they are not are not compromised by self-tolerance (as opposed to TAA which are associated with overexpression or otherwise expressed on non-mutated cells); and/or 2) they identify a cell displaying them on their surface as having dysregulated phosphorylation. Thus, post-translationally-modified phosphopeptides that are differentially displayed on cancer cells and derived from source proteins objectively linked to cellular transformation and metastasis allow for more extensive anti-tumor responses to be elicited following vaccination. Peptides are, therefore, better immunogens in peptide-based vaccines, as peptides are derived from proteins involved with cellular growth control, survival, or metastasis and alterations in these proteins as a mechanism of immune escape can interfere with the malignant phenotype of tumors.

As such, the presently disclosed subject matter also relates in some embodiments to methods for identifying peptides for use in immunotherapy which are displayed on transformed cells but are not substantially expressed on normal tissue in general or in the thymus in particular. In some embodiments, peptides bind the MHC class I molecule more tightly than their non-phosphorylated native counterparts. Moreover, such peptides can in some embodiments have additional binding strength by having amino acid substitutions at certain anchor positions. In some embodiments, such modified peptides can remain cross-reactive with TCRs specific for native peptide MHC complexes. Additionally, it is envisioned that the peptides associated with proteins involved in intracellular signaling cascades or cycle regulation are of particular interest for use in immunotherapy. In some cases, the TCR binding can specifically react with the phosphate groups on the peptide being displayed on an WIC class I molecule.

In some embodiments, the method of screening peptides for use in immunotherapy, e.g., in adaptive cell therapy or in a vaccine, involves determining whether the candidate peptides are capable of inducing a memory T cell response. The contemplated screening methods can include providing peptides, e.g., those disclosed herein or those to be identified in the future, to a healthy volunteer and determining the extent to which a peptide-specific T cell response is observed. In some embodiments, the extent to which the T cell response is a memory T cell response is also determined. In some embodiments, it is determined the extent to which a TCM response is elicited, e.g., relative to other T cell types. In some embodiments, those peptides which are capable of inducing a memory T cell response in health and/or diseased patients are selected for inclusion in the therapeutic compositions of the presently disclosed subject matter.

In some embodiments, the presently disclosed subject matter provides methods for inducing a peptide-specific memory T cell response (e.g., T_{CM}) response in a patient by providing the patient with a composition comprising the peptides disclosed herein. In some embodiments, the compositions are those disclosed herein and are provided in a dosing regimen disclosed herein.

In some embodiments, the presently disclosed subject matter relates to methods for determining a cancer disease prognosis. These methods involve providing a patient with peptide compositions and determining the extent to which the patient is able to mount a peptide specific T cell response. In some embodiments, the peptide composition contains peptides selected in the same substantially the same manner that one would select peptides for inclusion in a therapeutic composition. If a patient is able to mount a significant peptide-specific T cell response, then the patient is likely to have a better prognosis than a patient with the similar disease and therapeutic regimen that is not able to mount a peptide-specific T cell response. In some embodiments, the methods involve determining whether the peptide specific T cell response is a T_{CM} response. In some embodiments, the presence of a peptide-specific T cell response as a result of the presently disclosed diagnostic methods correlates with an at least or about 5, 10, 15, 20, 25, 30, 35, 40, 45, 50, 55, 60, 65, 70, 75, 80, 85, 90, 95, 100, 125, 150, 175, 200, 250, 300, 400, 500, or more percent increase in progression free survival over standard of care.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Example 1

To identify naturally processed tumor-associated phosphopeptides, affinity-isolated HLA-A*0201 (HLA-A2) and HLA-B*0702 (HLA-B7) peptide complexes were recovered from four (4) primary chronic lymphocytic leukemia (CLL) tumor samples, a primary acute lymphoblastic leukemia (ALL) sample, a primary acute myeloid leukemia (AML) sample, normal splenic T and B-cells, normal bone marrow cells (BM), and the EBV transformed, cultured B-lymphoblastoid cell line JY. Collectively, ten (10) HLA-A2-restricted and 85 HLA-B7-restricted phosphopeptides were identified from these samples. Of these, 8/10 A2 and 60/85 B7 phosphopeptides were not observed on the normal samples.

Next, a modified ELISpot was employed assay to assess the immune responses, exhibited by 10 HLA A2⁺ and 10 HLA B7⁺ typed healthy blood donors to synthetic versions of the 10 HLA A2 and 85 HLA B7 phosphopeptides detected on the leukemia tumors. Peripheral blood mononuclear cells (PBMCs; 1×10⁶ cells) isolated from fresh blood were suspended in AIM-V media (10% human serum) without the addition of stimulatory cytokines (IL-2) and then placed in ELISpotPRO plates containing 96 wells precoated with IFN-γ monoclonal antibody, mAb 1-D1K, (product code: 3420-2APW-2 from Mabtech). Activated CD8⁺ T cells secrete IFN-γ. Individual phosphopeptides (10 μg/ml) were added to each well and the plate was then placed at 37° C. in a CO₂ incubator for either 24 hours or 7 days. Many MHC peptides bind with low affinity to the MHC molecule on T-cells (or any other cells) and dissociate once they get to the cell surface. Empty MHC molecules on the cell surface are thus available for capture of peptides added exogenously. Once loaded, the resulting MHC complexes become targets for the corresponding peptide specific CD8+ cells in donor PBMCs.

Locations of individual activated CD8⁺ T cells appear as dark spots following a 15 minute reaction of alkaline phosphatase conjugate (Mabtech) with 5-bromo-4 chloro-3-indole phosphate and NBT and are counted by using an automated reader (AID-Diagnostika). Results from a subset of the 85 HLA B7 peptides are shown in Table 3 and are displayed as the number of spot-forming cells (SFC) per 10⁶ PBMCs. Note that T-cells from numerous healthy donors respond to phosphopeptides detected on AML but not on healthy B- or T-cells. Of the 79 HLA B7 peptides tested, 12, 19, and 30 stimulated an immune response in 6 or more, 4 or more, and 3 or more healthy donors, respectively.

It is important to note that the magnitude of the observed memory T-cell responses to the tumor phosphopeptides were comparable to that observed for memory T-cell responses to unmodified peptides derived from common virus proteins.

### Example 2

White blood cells (90% T-cells) were collected from a healthy blood donor (homozygous for HLA A*0201 and B*0702) and expanded in culture to 8×10⁸ cells. Half of this sample was treated for 4 hours with the PP2A/PP1 inhibitor, calyculin, and the other half was not. MHC peptides from both samples were isolated by the standard protocol (see e.g., Zarling et al., 2006), enriched for phosphopeptide neoantigens by IMAC, and analyzed by nano-flow HPLC interfaced to ETD mass spectrometry. The number of Class I MHC phosphopeptides detected and sequenced on the calyculin treated and untreated samples were 139 and 39, respectively. One hundred Class I MHC phosphopeptides were uniquely presented on the cell surface as a result of PP2A/PP1 phosphatase inhibition. Forty five of these peptides had previously been found on multiple cancers and on the EBV (Epstein Barr Virus) immortalized B-cell, lymphoblastoid cell line, JY. See Table 3.

TABLE 3
Phosphopeptides Expressed on PP2A-inhibited
Healthy White Blood Cells
HLA A*02

| SEQ | | No | With | Found on |
|---|---|---|---|---|
| ID | | Inhib- | Inhib- | Other |
| NO. | Sequence | itor | itor | Cancers |
| | AVVsPPALHNA | No | Yes | O, M, JY, H |
| | GLDsGFHSV | No | Yes | H |
| | ILDsGIYRI | No | Yes | O, M, JY |
| | KAFsPVRSV | No | Yes | O, C, E, |
| | | | | H, L |
| | KIFsGVFVKV | No | Yes | H, JY |
| | KIGsIIFQV | No | Yes | O, H |
| | KLFPDtPLAL | No | Yes | O, C, M, |
| | | | | JY, H, L |
| | KLIDRTEsL | No | Yes | C, H, C, JY |
| | LLDFGSLSNLQV | No | Yes | M |
| | KLMsPKADVKL | No | Yes | O, M, H, JY |
| | KVAsLLHQV | No | Yes | O, H, JY |
| | RITsLIVHV | No | Yes | O |
| | RLDsYVRSL | No | Yes | O, E, M, |
| | | | | JY, H |
| | RLIsQIVSSI | No | Yes | CML |
| | RLLsPQQPAL | No | Yes | H |
| | RMLsLRDQRL | No | Yes | O |
| | RQAsLSISV | No | Yes | O, H, JY |
| | RQDsTPGKVFL | No | Yes | O, C, M, |
| | | | | JY, H |
| | RQIsQDVKL | No | Yes | O, C, E, |
| | | | | M, JY, H |
| | SLSsLLVKL | No | Yes | O |
| | TLAsPSVFKST | No | Yes | M, O, JY |
| | VLYsPQMAL | No | Yes | O, H |
| | VMFRIPLASV | No | Yes | O, M, JY |

### Example 3

From an HBV induced tumor sample that expressed HLA B*07, 133 class I MHC phosphopeptides were identified. Fifty-five of these peptides had been previously on two or more of the following cancers, melanoma, colorectal cancer, ovarian cancer and multiple leukemias. Twenty-five of the peptides had been tested earlier and found to be recognized by central memory T-cells. All fifty-five of these class I MHC phosphopeptides were also found on the HBV infected tissue that surrounded the tumor.

Similar results were obtained from the analysis of HLA A*03 phosphopeptides expressed on two liver tumors, one caused by HBV and the other by HCV. Seventeen HLA A*03 phosphopeptides that were found previously on multiple other cancers were also detected on the two liver cancers but not on normal cells. These same 17 phosphopeptides were also expressed on the surgically removed tissues that surrounded the tumors but were infected with HCV and HBV, respectively. These findings provided strong evidence that many class I MEW phosphopeptides expressed on cancers should also be found on virus infected cells and can thus be used as targets for immunotherapy of both types of disease.

Additional HBV and HCV surgical tumor samples and their surrounding tissues are tested in order to characterize MEW phosphopeptides presented by all the major Class I, MEW alleles; A*01, A*02, A*03, B*07, B*27, B*44, C*04, C*05, C*06, and C*07.

TABLE 4
HLA-A*03

| | | On Adja- | On Adja- | | |
|---|---|---|---|---|---|
| | | cent | cent | | |
| | | Healthy | Healthy | | |
| | | Tissue | Tissue | On | Found |
| SEQ | | Infected | Infected | HCC | on |
| ID | | With | With | Tumor | Other |
| NO | Peptide | HCV | HBV | 1, 2 | Cancers |
| | GIMSPLAKK | Y | Y | Y | C, H |
| | KLPsPAPARK | Y | Y | Y | C, H |
| | KLRsPFLQK | N | Y | Y | M, L, H |
| | KMPTtPVKAK | N | Y | Y | M, C, H |
| | KTPTSPLKMK | Y | Y | Y | M, L, H |
| | RAKsPISLK | Y | Y | Y | C, M, |
| | | | | | L, H |
| | RSYsYPRQK | Y | Y | Y | M, H |
| | RTAsFAVRK | Y | Y | Y | M, H |
| | RTASPPPPPK | Y | Y | Y | M, L, H |
| | RVKtPTSQSYR | Y | Y | Y | M, H |
| | RVLsPLIIK | Y | Y | Y | C, M, H |
| | RVYSPYNHR | Y | Y | Y | C, L, |
| | | | | | M, H |
| | SVKsPVTVK | Y | Y | Y | C, M, H |
| | SVRRsVLMK | Y | Y | Y | C, M, H |
| | TLLAsPMLK | Y | Y | Y | |

TABLE 5
HLA-B*07

| | | | On | | On | |
|---|---|---|---|---|---|---|
| | | | Adjacent | | Adjacent | |
| | | On | Healthy | On | Healthy | |
| SEQ | | HCC | Tissue | HCC | Tissue | |
| ID | | Tumor | infected | Tumor | with | Found on Other |
| NO | Peptide | 1 | with HBV | 2 | Adenoma | Cancers |
| | APDsPRAFL | Y | Y | Y | N | C, H |
| | APRRYSSSL | Y | Y | Y | N | C, M, O, L, H |
| | FPRRHsVTL | Y | Y | Y | N | M, C, L, H |
| | GPRPGSPSAL | Y | Y | Y | N | O, M, H |
| | KPASPKFIVTL | Y | Y | Y | N | C, M, O, L, H |
| | KPRPPPLSP | Y | Y | Y | N | C, M, H |
| | RPDVAKRLsL | Y | Y | Y | N | C, H, |
| | RPFSPREAL | Y | Y | Y | N | C, M, O, L, H |
| | RPIsPGLSY | Y | Y | Y | N | E, M, O, C, H |
| | RPKsPLSKM | Y | Y | Y | N | C, H |
| | RPKsVDFDSL | Y | Y | Y | N | C, M, H |
| | RPNsPSPTAL | Y | Y | Y | N | M, O, L, H |
| | RPPsPGPVL | Y | Y | Y | N | M, O, L, H |
| | RPRARsVDAL | Y | Y | Y | N | C, M, O, H, |
| | RPRPHsAPSL | Y | Y | Y | N | M, O, L, C, H |
| | RPRPVsPSSLL | Y | Y | Y | N | M, H |
| | RPRsAVEQL | Y | Y | Y | N | C, H |
| | RPRsISVEEF | Y | Y | Y | N | M, C, H |
| | RPRSL(ss)PTVTL | Y | Y | Y | N | O, M, H |
| | RPRsPNMQDL | Y | Y | Y | N | C, H |
| | RPRsPPGGP | Y | Y | Y | N | C, H |
| | RPRsPRQNSI | Y | Y | Y | N | C, E, L, M, O, H |
| | RPRsPTGPSNSF | Y | Y | Y | N | C, O, M, H |
| | RPRsPTGPSNSFL | Y | Y | Y | N | M, O, H |
| | RP(SS)LPDL | Y | Y | Y | N | M, O, L, H |
| | RPTsFADEL | Y | Y | Y | N | E, H |
| | RPTSRLNRL | Y | Y | Y | N | C, M, B, H, L |
| | RPVsPFQEL | Y | Y | Y | N | E, M, O, L, C, H |
| | RPYSPPFFSL | Y | Y | Y | N | M, O, L, H |
| | SPAsPKISL | Y | Y | Y | N | M, O, L, H |
| | SPFKRQLsL | Y | Y | Y | N | C, M, O, H |
| | TPRsPPLGL | Y | Y | Y | N | C, M, O, H, JY |
| | TPRsPPLGLI | Y | Y | Y | N | C, M, O, H, Jy |
| | VPRPERRsSL | Y | Y | Y | N | C, H |

### Example 4

### Identification of Class I MHC Phosphopeptide Antigens Presented by Cells Infected with Human Papillomavirus (HPV) and the Epstein Barr Virus (EBV)

To identify MHC class I phosphopeptide antigens presented on head-neck and cervical cancers, both of which are caused by the HPV virus, samples of the above tumors and the surrounding healthy or HPV infected tissue are analyzed. Approximately 50 tumor samples are employed to identify phosphopeptides presented by the ten major class I MHC alleles on the above cancers.

Also characterized are class I MHC phosphopeptide antigens that are presented on (a) normal endothelial cells and (b) endothelial cells transduced to express the HPV (type 16) E7 accessary protein that binds and inactivates the pRb protein. Keratinocytes are immortalized with a retroviral vector that encodes the human telomere reverse transcriptase hTERT as described in Dickson et al., 2000, which allows the cells to maintain telomere length and grow to numbers that are sufficient for these experiments. Anticipated results for these experiments are as follows. Sample (a) should present only a small number of phosphopeptides usually found on normal cells. Sample (b) should present the phosphopeptides found on sample (a) plus many of the phosphopeptide antigens already discovered on HPV infected tissue and on multiple types of cancer.

With respect to the Epstein Barr Virus (EBV), this virus causes Hodgkin's lymphoma, Burkitt's lymphoma, and both gastric cancer and nasopharyngeal carcinoma. Presentation of class I MEW phosphopeptides on normal B-cells and B-cells transfected with DNA for the EBV protein EBNA-3C (also known as EBNA 6) with and without immortalization by hTERT are performed. EBNA-3c mediates ubiquitination of and degradation of pRb, which in turn leads to high levels of transcription and upregulation of CIP2A. Anticipated results of these two experiments should be very similar to that described herein above for treatment T-cells with and without the PP2A inhibitor calyculin.

### Example 5

### Identification of Class I MHC Phosphopeptide Antigens Presented by Cells Infected with HIV

Beads covalently linked to an anti-HLA class I antigen antibody (W6-32; Abcam, Cambridge, United Kingdom) are employed to affinity purify class I MHC peptide complexes from three separate cultures of 5×10-CD4 T-cells. Sample #1 is MHC phosphopeptides from normal CD4 T-cells, Sample #2 are infected with HIV, and Sample #3 are infected with a strain of HIV that lacks the Nef protein. The Nef protein is expexted to suppress presentation of class I HLA-A, partially suppress HLA-B, and have no effect on HLA-C and E. Sample #1 is expected to show low levels of multiple phosphopeptides but not express any that have already been documented as being unique to multiple cancers. Sample #2 is expected to be devoid of HLA-A phosphopeptides, to show low levels of HLA B phosphopeptides (both those on sample #1 and new ones that are unique to the infection), and to show abundant HLA-C phosphopeptides that include those on the normal cells plus new ones that are also found on multiple cancers. Sample #3 is expected to present abundant phosphopeptides on all three HLA types: A, B, and C. Many of these are anticipated to be identical to those that have already been found on multiple cancers.

### Example 6

### Identification of Class I MEW Phosphopeptide Antigens on Cells Infected with MCPyV

Cells infected with MCPyV are expected to present the same MHC class I phosphopeptides as has been found on multiple tumors because the viral protein, LT, represses transcription of p53, a truncated version of LT inactivates pRb, and the ST protein inhibits PP2A. The MHC phosphopeptides presented on NSG mouse xenografts of normal human dermal fibroblast cells, with and without immortalization by hTert as described above, and both, with and without, transfection of the three viral proteins is tested. Only the samples transfected with the polyomavirus proteins are expected to present phosphopeptides observed on multiple tumors.

### Example 7

### Identification of Class I WIC Phosphopeptide Antigens on Cells Infected with H. pylori and Fn

Experiments to characterize WIC class I phosphopeptide antigens that are expressed by cells infected with the bacterium H. pylori are performed on human-derived normal fundic gastric organoids (huFGOs) and human-derived tumor gastric organoids (huTGOs) as described in Steele et al., 2019. Both samples are obtained with appropriate permission from healthy and diseased tissues surgically removed from patients. One sample of huFGOs (normal) is transfected with the gene for the H. pylori CagA protein. Xenografts of the three organoid samples (a) HuFGO, (b) HuFGO with transfected CagA protein, and (c) huTGO all on NSG mice are prepared according to Steele et al., 2019. Because the H. pylori protein CagA binds to E-cadherin and displaces β-catenin, it is anticipated that CIP2A is overexpressed in samples (b) and (c), that it inhibits PP2A, and thus generates many of the class I MHC phosphopeptide antigens that have already been found on multiple cancers. Few, if any, phosphopeptide antigens are presented on the normal sample (a).

Experiments to characterize class I MHC phosphopeptide antigens that are expressed by cells that are infected with gram negative anaerobe Fusobacterium nucleatum (Fn) are performed using NSG mouse xenografts of (A) surgically resected human colorectal cancer tissue, (B) healthy adjacent tissue (devoid of the Fn bacterium), and (C) healthy adjacent tissue that has been infected with Fn. The Fn protein FadA and the Fn lipopolysaccharide have been reported to activate β-catenin signaling that usually upregulates transcription, which results in generation of CIP2A and inhibition of PP2A. Accordingly, samples A and C present many of the Class I MHC phosphopeptide antigens that have already been found on multiple cancers, and few, if any, phosphopeptide antigens are found on sample B.

### Discussion of the EXAMPLES

A goal of the presently disclosed subject matter is to identify class I MHC phosphopeptides that (a) result from dysregulated cell signaling pathways in cancer, (b) are uniquely expressed on tumors but not normal cells, (c) are found on multiple types of cancer, (d) are recognized by central memory T-cells in PBMC from healthy blood donors, and (e) trigger killing by cytotoxic T-cells. More than 2000 class I MEW phosphopeptides presented by multiple HLA alleles (A*01, 02, 03, B*07, 44, 27, and C*04, C*05, 06, and 07) on leukemias (AML, ALL, and CLL), melanoma, breast, ovarian, colorectal, esophageal, and hepatocellular cancers have been identified (see e.g., U.S. Patent Application Publication No. 2015/0328297; 2016/0000893; 2019/0015494; 2019/0374627; and U.S. Pat. No. 9,561,266). Of these peptides, 70-80 percent are not on the corresponding normal cells or tissue and more than 1200 are found on multiple types of cancer. Of those tested, about 50% are recognized by central memory T-cells.

These results provided evidence that onset of cellular transformation occurs frequently in healthy individuals but can be controlled by an immune system response to class I MHC phosphopeptides. Leukemia patients, who are in control of their disease, usually have strong T-cell responses to class I MEW phosphopeptides. Late stage AML patients often lack phosphopeptide specific immunity but can recover it following stem cell transplantation. Particularly noteworthy is the finding that the same tumor specific phosphopeptides are found on multiple (3 to 8) different types of cancer. In short, it appears that a small cocktail of class I phosphopeptides could be used to treat all of the above cancers, particularly when used in combination with one or more check-point blockade inhibitors (e.g., anti-PD1, anti-PDL-1, anti-CTLA-4, etc.) that upregulate the immune response in the tumor microenvironment. Thus, class I MEW phosphopeptides are likely to be excellent targets for multiple cancer immunotherapy strategies.

An exemplary approach for prioritizing the phosphopeptides in the clinical trials could be as follows: select the phosphopeptide targets that (a) are presented by one of the 6 most common HLA alleles; (b) are detected on multiple tumor types and thus can be used to treat multiple cancers; (c) are not detected on healthy tissue; (d) are recognized by central memory T-cells from healthy blood donors that do not have autoimmune disease (which means that these peptides will likely elicit a strong immune response to the tumor and not to any other healthy tissue); (e) are derived from a parent protein that is associated with a known cancer signaling pathway; (f) are presented on the tumor at the level of 25-100 copies/cell; and (g) have a binding affinity to the MHC molecule that is in the low nanomolar range. For microbial infections, a similar approach can be taken.

Besides the identification of cancer specific class I MHC phosphopeptides, class I MHC peptides on tumors that result from dysregulation of two additional, critical cell signaling processes—methylation on Arg and Lys and O-GlcNAcylation on Ser and Thr—have also been identified. Both signaling pathways exhibit cross talk with phosphorylation and all three pathways play major roles in the transformation process. In leukemia cells, for example, 74 O-GlcNAcylated and 44 methylated Arg (monomethyl, sym-, and asym-dimethyl) containing class I MHC peptides have been characterized. Many of these peptides are also recognized by memory T-cells in PBMC from healthy blood donors. Thus, it is possible to enrich and detect tumor-specific, methylated, phosphorylated, and O-GlcNAcylated peptides from the same tumor sample of about 1-5×10⁷ cells (˜1-8 mm³ of tissue).

The presently disclosed subject matter also relates to compositions and methods for identifying post-translationally modified, class I MHC peptides that are uniquely presented on microbially infected cells. Significantly, new antigens that can be used for immunotherapy of multiple viral infections have been identified, as have antigens that are common to both cancer and specific microbial infections. Discovery of post-translationally modified antigens that are common to cancer and one or more microbial infections suggests that some of the central memory T-cells that recognize and kill cancer cells might have been generated from an earlier response to a infection rather that from immune surveillance of cancer. Discovery of such post-translationally modified antigens thus opens the door to the development of vaccination protocols against both diseases.

While not wishing to be bound by any particular theory of operation, the presently disclosed subject matter is supported by evidence that many class I MHC phosphopeptides are generated by dysregulated signaling pathways that occur in cancer. Since these peptides are not found on normal cells in the thymus or lymph nodes, tolerance to these antigens (deletion of high avidity T-cells) is not likely to develop. If the kinase or target protein is also required for the transformation process, angiogenesis, metastasis, or another critical tumor function, the tumor escapes by mutation or gene deletion without compromising tumor survival is also unlikely.

Development of a technology for the enrichment and sequence analysis of class I and class II phosphopeptides at the attomole level has also occurred. Critical improvements to the basic immobilized metal affinity chromatography (IMAC Fe⁺³) enrichment protocol include: (a) use of homemade 150 μm i.d.×360 μm o.d. fused silica, nanoflow HPLC column (5 μm C18 beads) to clean up the sample before the peptide esterification step; (b) use of shorter and smaller diameter IMAC columns (3″ of packing in 50 μm i.d. fused silica); (c) much longer equilibration times for loading FeCl₃ on the chelating resin to eliminate nonspecific binding of multiply charged, non-phosphorylated peptides to unoccupied, negatively-charged, metal-binding sites; (d) use of multiple phosphopeptide internal standards to quantitate recoveries for each step in the protocol and to act as carriers to minimize loss of low level class I phosphopeptides; and (e) development of an improved neutral loss algorithm that optimizes detection of phosphoric acid loss in the CAD spectrum of a phosphopeptide parent ion. All class I MHC peptide samples are screened by using 1×10⁷ cell equivalents (material from 10 million cells) and then IMAC enrichment is performed on material from 1-2×10⁸ cells. Class I MHC phosphopeptides are sequenced at the 5-50 attomole level (less than 1 copy/cell). Total phosphopeptide quantities in the sample seldom exceed 100 fmol and yet typical recoveries are in the range of 50-60%.

Additionally, technology for the enrichment and sequence analysis of class I MHC O-GlcNAcylated peptides at the attomole level has also been developed. Here, an innovation involves esterification of the O-GlcNAc moiety with immobilized aminophenylboronic acid under anhydrous conditions. POROS20 AL beads are covalently linked to aminophenylboronic acid with sodium cyano borohydride. Cleaned-up samples of MHC peptides are then taken to dryness, dissolved in anhydrous DMF, and allowed to react with the derivatized beads for 2 hours at room temperature. Solvent is then removed and the O-GlcNAcylated peptides are released on treatment of the beads with 0.1% acetic acid.

Additionally, mass spectrometry instrumentation and protocols that facilitate sequence analysis of post-translationally modified peptides at the attomole level have been developed. Key innovations here include: (a) development of nanoflow (60 nl/min) chromatography on homemade columns with built in laser pulled tips for highly efficient electrospray ionization; (b) butt-connection of additional columns to perform efficient sample clean-up and IMAC for enrichment of phosphopeptides; (c) the use of Electron Transfer Dissociation (ETD) Mass Spectrometry (Syka et al., 2004) for efficient dissociation of posttranslationally modified peptides (without loss of the modification); and (d) development of a front-end ETD ion source that allows multistep accumulation of ion current from ETD fragments so as to further enhance sensitivity (Earley et al., 2013) and facilitate sequence analysis of phosphopeptides at the level of 5-10 attomoles.

Additionally, an improved ELISpot assay was employed for detection of central memory, T-cell recall-responses to post translationally modified, class I MHC, tumor antigens in PBMC from healthy blood donors. This assay dramatically reduced the time and effort (weeks to days) required to select the best class I MHC antigens for use in cancer immunotherapy (Hunt et al., 2007).

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
Abelin et al. (2015) Nature Protocols 10:1308-1318.
Akamatsu et al. (1997) Bioorg Med Chem 5:157-163.
Altschul et al. (1990) J. Mol. Biol. 215:403-410.
Altschul et al. (1997, Nucleic Acids Res. 25:3389-3402.
Biggar & Li (2015) Nat Rev Mol Cell Biol 16:5-17.
Bodanszky & Bodanszky (1984) in The Practice of Peptide Synthesis, Springer-Verlag, New York, New York.
Brahmer et al. (2012) N Engl J Med 366:2455-2465.
Caldwell et al. (2010) Oncogene 29:2831-42.
Cante-Barrett et al. (2014) Oncogene 33:403-410.
Chothia et al. (1985) J Mol Biol 186:651-666.
Chothia et al. (1989) Nature 342:877-883.
Civan & Hann (2014) N A J Med Sci 7:8-16.
Clackson et al. (1991) Nature 352:624-628.
Clark et al. (1989) Eur J Immunol 19:381-388.
Cobbold et al. (2013) MHC Class I-Associated Phosphopeptides are theTargets of Memory-Like Immunity in Leukemia, Sci Transl Med 5:203ra125.
Cole et al. (1985) Proc Natl Acad Sci USA 82:859.
Cote et al. (1983) Proc Natl Acad Sci USA 80:2026.
Cox et al. (1994) Science 264:716-719.
Current Protocols in Immunology (1990-2019) Wiley Interscience, Hoboken, New Jersey, United States of America.
De Queiroz et al. (2014) Frontiers in Oncology 4(132):1-10.
Dickson et al. (2000) Mol Cell Biol 20;1436-1447.
Diehl & Schaal (2012) Viruses 5:3192-3212.
Dudley et al. (2008) J Clin Oncol 26(32):5233-9.
Dunn et al. (2004) Annu Rev Immunol 22:329-360.
Earley et al. (2013) Anal Chem 85(17):8385-8390.
Fishwild et al. (1996) Nature Biotechnol 14:845.
Gattinoni et al. (2006) Nature Rev Immunol 6:383-393.
GB Patent Application No. 2249310A
Gennaro (1990) Remington's Pharmaceutical Sciences, Mack Publishing, Easton, Pa., United States of America.
Gross & Mienhofer (1981) The Peptides, vol. 3 Academic Press, New York, N.Y., United States of America, pp. 3-88.
Gubin et al. (2015) J. Clin Invest 125:3413-3421.
Guergnon et al. (2011) Biochim Biophys Acta 1812:1498-1507.
Haesen et al. (2014) Frontiers in Oncology 4:1-11.
Hanahan & Weinberg (2011) Cell 144:646-674.
Hodi et al. (2010) N Engl J Med 363:711-723.
Hogan et al. (1998) Cancer Res. 58:5144-5150.
Hoogenboom et al. (1991) Nucleic Acids Res 19:4133.
Hunt et al. (1992a) Science 255:1261-1263.
Hunt et al. (1992b) Science 256:1817-1820.
Hunt et al. (2007) J. Immunol 179:2690-71.
Jones et al. (1986) Nature 321:522-525.
Kabat et al. (1987) Sequences of Proteins of Immunological Interest, National Institute of Health, Bethesda, Md., United States of America.
Karlin & Altschul (1990) Proc. Natl. Acad. Sci. USA 87:2264-2268.
Karlin & Altschul (1993) Proc. Natl. Acad. Sci. USA 90:5873-5877.
Khanna et al. (2013) Cancer Res 73:6548-6553.
Kiely & Kiely (2015) Cancers 7:648-669.
Kittlesen et al. (1998) J Immunol 60:2099-2106.
Kohler & Milstein (1975) Nature 256:495.
Kozbor et al. (1983) Hybridoma, 2:7.
Lanier et al. (2010) Bioorganic & Medicinal Chemistry 18:573-579
LeFranc & LeFranc (2001) The T Cell Receptor Factsbook, ISBN 0-12-441352-8, Academic Press, San Diego, Calif., United States of America.
Liddy et al. (2012) Nature Med 18(6):980-987.
Lonberg & Huszar (1995) Intl Rev Immunol 13:65.
Lonberg et al. (1994) Nature 368:856.
Lynch et al. (2012) J Biol Chem 287(14):11070-81.
Mackensen et al. (2000) Int J Cancer 86:385-392.
Malaker et al. (2017) J Proteome Res 16:228-237.
Marino et al. (2015) J Am Chem Soc 137:10922-10925.
Marino et al. (2017) J Proteome Res 16:34-44.
Marks et al. (1991) J Mol Biol 222:581-597.
Marks et al. (1992) J Biol Chem 267:16007.
Martin et al. (2000) Anal Chem 72:4266-4274.
Matsushita et al. (2012) Nature 16;482:405-410.
McGranahan et al. (2016) Science 351:1463-1469.
Melief (2009) J Med Sci 2:43-45.
Mihara et al. (2001) Clin Immunol 98:319.
Morales et al. (2000) Nucl Med Biol 27:199.
Morgan & Gainor (1989) Ann Repts Med Chem 24:243-252
Murphy (2012) Janeway's Immunobiology, 8th Edition, Garland Science, New York, N.Y., United States of America.
Neuberger (1996) Nature Biotechnol 14:826.
Novotny & Haber (1985) Proc Natl Acad Sci USA 82:4592-4596.
Otaka et al. (1995) Tetrahedron Lett 36:927-930.
Pardoll (2012) National Reviews of Cancer, Focus on Tumor Immunology & Immunotherapy, 254, April 2012, Volume 12.
Parmiani & Anichini (2006) J Hepatol 45:178-181.
Nature Rev Cancer 12:252-264.
PCT International Patent Application Publication Nos. WO 1994/02602; WO 1996/33735; WO 1996/34096; WO 1999/54464; WO 1999/61065; WO 2002/070006; WO 2004/106380; WO 2004/110486; WO 2011/149909; WO 2013/177593; WO 2014/036562; WO 2014/036562; WO 2014/039675; WO 2014/039675; WO 2014/093855; WO 2015/034519; WO 2015/120036; WO 2017/027403; WO 2017/192969.
Perrotti & Neviani (2013) Lancet Oncology 14:e229-e238.
Pim et al. (2005) Oncogene 24:7830-7838.
Pippa et al. (2014) Leukemia 28:1915-1918.
Porter et al. (2011) N Engl J Med 365(8):725-33.
Presta (1992) Proc Natl Acad Sci USA 89:4285-4289.
Prieto et al. (2015) Nat Rev Gastroenterol Hepatol 12:681-700.
Rapp & Kaufmann (2004) Int Immunol 16(4):597-605.
Remington & Gennaro (1995) Remington: The Science and Practice of Pharmacy, Mack Publishing, Easton, Pa., United States of America.
Restifo et al. (2012) J Exp Med 12:269-281.
Richards et al. (1999) Cancer Res 59:2096.
Riechmann et al. (1988) Nature 332:323-327.
Rowe et al. (eds.) (2006) Handbook of Pharmaceutical Excipients, 5^{th} ed. Pharmaceutical Press, London, United Kingdom.
Ruvolo (2006) BBA Clinical 6:87-99.
Sage et al. (2016) Viruses 8:1-19.
Sandborn et al. (2001) Gastroenterology 120:1330-1338.
Sangodkar et al. (2016) FEBS J 283:1004-1024.
Schreiber et al. (2011) Science 331:1565-1570.
Schumacher & Schreiber (2015) Science 348:69-73.
Shamay et al. (2012) J Virology 86:5179-5191.
Shinkura et al. (1998) Anticancer Res 18:1217.
Slawson & Hart (2011) Nat Rev Cancer 11(9):678-84.
Smyth et al. (1992) Tetrahedron Lett 33:4137-4140.
Soderholmm et al. (2016) Mol Cell Proteomics 15:3203-3219.
Sontag & Sontag (2014) Frontiers in Mol Neuroscience 7:1-10.
Spatola (1983) Chemistry and Biochemistry of Amino Acids, Peptides and Proteins, Vol. VII (Weinstein ed.) Marcel Dekker, New York, N.Y., United States of America
Steele et al. (2019) Cellular and Molecular Gastroenterology and Hepatology 7;161-184.
Stewart et al. (1984) in Solid Phase Peptide Synthesis, 2nd Edition, Pierce Chemical Company, Rockford, Ill., United States of America.
Stopa et al. (2015) Cellular and Molecular Life Sciences 72:2041-2059.
Strickley (2004) Pharm Res 21:201-230.
Syka et al. (2004) Proc Natl Acad Sci USA 101(26):9528-33.
Topalian et al. (2012) N Engl J Med 366:2443-2454.
U.S. Patent Application Publication Nos. 2002/0119149; 2004/0202657; 2008/0153112; 2009/0117102; 2009/0226474; 2010/0297158; 2013/0259883; 2013/0259883; 2015/0328297; 2016/0000893; 2017/0029484; 2018/0066017; 2019/0015494; 2019/0374627.
U.S. Pat. Nos. 4,361,539; 4,816,567; 5,225,539; 5,545,806; 5,545,807; 5,569,825; 5,625,126; 5,633,425; 5,661,016; 5,789,543; 5,916,771; 5,939,598; 5,968,509; 6,207,718; 6,706,265; 6,750,325; 8,012,932; 8,119,984; 8,211,436; 8,692,187; 9,171,707; 9,279,011; 9,279,011; 9,561,266; 10,281,473.
Van Wauve (1980) J Immunol 124:2708-2718.
Verhoeyen et al. (1988) Science 239:1534-1536.
Vesely et al. (2011) Annu Rev Immunol 29:235-271.
Wang et al. (2010) Sci Signal 3(104):ra2.
Wells et al. (2004) J Biol Chem 279(37):38466-38470.
Welters et al. (2004) Vaccine 23(3):305-311.
Whittaker et al. (2010) Oncogene 29:4989-5005.
Wojcechowskyj et al. (2013) Cell Host & Microbe 13:613-623.
Yadav et al. (2014) Nature 515:572-576.
Yamashita et al. (2011) J Gastroenterol Hepato! 26:960-964.
Yang & Bedford (2013) Nat Rev Cancer 13:37-50.
Yee et al. (2002) Proc Natl Acad Sci USA 99:16168-16173.
Yenari et al. (1998) Exp Neurol 153:223.
Yenari et al. (2001) Neurol Res 23:72.
Zarling et al. (2006) Proc Natl Acad Sci USA 103:14889-14894.
Zhang & Claret (2012) Enzyme Res 2012:Article 659649.
Zugel (2001) Infect Immun 69(6):4164-4167.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

TABLE 6
Exemplary Peptides of the Presently
Disclosed Subject Matter

| SEQ | | | |
|---|---|---|---|
| ID | | | |
| NO: | SEQUENCE | | |
| 8 | AAsDTERDGLA | 152 | APYRGQLAsPSSQ |
| 10 | AAsPGAPQM | 154 | ARAsPRLHFL |
| 12 | ADsGEGDFLAEGGGVR | 155 | ARFsGFYSM |
| 13 | AEAPLPsPKL | 155 | ARFSGFYsM |
| 20 | AEFPSSGsNSVL | 162 | ARFsPKVSL |
| 28 | AELsPVEQKL | 163 | ARGsLRRLL |
| 30 | AENARSAsF | 167 | ARVsPSTSY |
| 31 | AENsPTRQQF | 174 | ASEsPSSLIFY |
| 36 | AEQGsPRVSY | 181 | ASLsRPLNY |
| 37 | AERtPELVEL | 182 | ASMsPGHPTHL |
| 44 | AGDsPGSQF | 186 | ASsPPDRIDIF |
| 46 | AIMRsPQMV | 188 | ASSsQIIHI |
| 49 | ALAAPsPPR | 189 | AtAGPRLGW |
| 51 | ALDsGASLLHL | 189 | AtAGPRLGw |
| 55 | ALGNtPPFL | 196 | ATIPRPFsV |
| 65 | ALMGsPQLV | 197 | ATMKRMLsL |
| 66 | ALMGsPQLVAA | 199 | ATYtPQAPK |
| 86 | AMDsPLLKY | 202 | AVILPPLsPYFK |
| 100 | APAsPLRPL | 204 | AVVsPPALHNA |
| 102 | APAsPRLL | 206 | AyAQPQTTTPLPAVSG |
| 105 | APDsPRAFL | 208 | AYGGLTsPGLSY |
| 106 | APDsPSKQL | 224 | DERLRINsL |
| 120 | APRKGsFSAL | 227 | DETERAYsF |
| 125 | APRNGsGVAL | 228 | DGRRtFPRI |
| 127 | APRRYsSSL | 231 | DLKRRsMSI |
| 133 | APRsPPPSRP | 235 | DLKSSKAsL |
| 137 | APSLFHLNtL | 245 | DQFERIKtL |
| 258 | DSsEEKF | 255 | DsFESIESY |
| 260 | DSsEEKFLR | 257 | DssEEK |
| 263 | DSVsPSESL | 437 | GAVsPVGEL |
| 269 | DVYSGtPTKV | 445 | GEAsPSHII |
| 280 | EELsPTAKF | 450 | GEIsPQREV |
| 297 | ELLPRRNsL | 454 | GELPTsPLHLL |
| 302 | EPKRRsARL | 464 | GETsPRTKI |
| 305 | EPRNSLPAsPAHQL | 465 | GETsPRTKITW |
| 308 | EPRsPSHSM | 469 | GGDsPVRL |
| 323 | FASPTsPPVL | 471 | GGPHFsPEHKEL |
| 324 | FATIRTAsL | 477 | GIDsPSSSV |
| 325 | FAVsPIPGRGGVL | 478 | GIFPGtPLKK |
| 328 | FAYsPGGAHGML | 479 | GIMsPLAKK |
| 341 | FKtQPVTF | 484 | GLDsGFHSV |
| 346 | FLDtPIAKV | 491 | GLIsPVWGA |
| 358 | FPAsPSVSL | 493 | GLLDsPTSI |
| 368 | FPLARQFsL | 503 | GLSsLSIHL |
| 369 | FPLDsPKTLVL | 504 | GLTsPGLSY |
| 373 | FPRRHsVTL | 422 | GPPYQRRGsL |
| 384 | FRFsGRTEY | 527 | GPRASsLLsL |
| 385 | FRGRYRsPY | 529 | GPRPGsPSAL |
| 386 | FRKsMVEHY | 533 | GPRSAsLL |
| 387 | FRRsPTKSSL | 535 | GPRSASLLsL |
| 389 | FRRsPTKSSLD | 538 | GPRsPKAPP |
| 394 | FRRsPTKSSLDY | 541 | GQLsPGVQF |
| 402 | FRYsGKTEY | 524 | GRKsPPPSF |
| 413 | FSIsPVRL | 554 | GRLsPAYSL |
| 418 | FSVAsPLTL | 565 | GRLSPVPVPR |
| 420 | FSYsPRLPL | 567 | GRQsPSFKL |
| 422 | FTDVNsILRY | 580 | GSDVsLTAcKV |
| 426 | FTKsPYQEF | 582 | GsGPEIFTF |
| 436 | GATTTAPsL | 583 | GSKsPISQL |
| 586 | GsPHYFSPFRPY | 584 | GsPHYFSPF |
| 587 | GsPIKVTL | 588 | GsPHYFSPFRP |
| 588 | GsPIKVTLA | 714 | IRFGRKPsL |
| 594 | GTIRSRsFIFK | 733 | ISIDsPQKL |
| 602 | GTVtPALKL | 735 | ISSsMHSLY |
| 603 | GTYVPSSPTRLAY | 738 | IsTSPSVAL |
| 605 | GVIsPQELLK | 738 | IStSPSVAL |
| 606 | GVIsPQELLKK | 738 | ISTsPSVAL |
| 609 | GYVQRNLSLVRG | 738 | IstSPSVAL |
| 622 | HPKRSVsL | 738 | IsTsPSVAL |
| 638 | HPYsPLPGL | 738 | IStsPSVAL |
| 652 | HRVsVILKL | 738 | IstsPSVAL |
| 654 | HRYsTPHAF | 740 | ISVsPLATSAL |
| 654 | HRYStPHAF | 755 | ITItPPDRY |
| 654 | HRYstPHAF | 785 | ITQGtPLKY |
| 654 | HRYsTPHAF | 762 | ITYMsPAKL |
| 656 | HTAsPTGMMK | 769 | IYQyIQSRF |
| 657 | HTFsPSPKL | 771 | IYYKsMPNL |
| 661 | HVYtPSTTK | 775 | KAFsPVR |
| 665 | IAKsPHSTV | 777 | KAFsPVRSV |
| 671 | IIHsLETKL | 777 | KAFsPVRSV |
| 674 | IISsPLTGK | 781 | KAKsPAPGL |
| 678 | ILDsGIYRI | 783 | KAPsPPPLL |
| 681 | ILKPRRsL | 784 | KAPsRQISL |
| 694 | IPAPPSsPL | 789 | KASPKRLsL |
| 698 | IPLSKIKtL | 790 | KAVsLFLc |
| 699 | IPRPLSLIGSTL | 794 | KEKsPFRET |
| 700 | IPRsPFKVKVL | 796 | KELARQIsF |
| 701 | IPRTPLsPSPM | 799 | KEMsPTRQL |
| 709 | IPVSSHNSL | 802 | KEQsPEPHL |
| 710 | IPYAPsGEIPK | 803 | KESSPLSSRKI |
| 711 | IQFsPPFPGA | 804 | KEStLHLVL |
| 813 | KIDsPTKVK | 805 | KEtPDKVEL |
| 817 | KIFsGVFVK | 811 | KIAsEIAQL |
| 818 | KIFsGVFVKV | 1036 | KPPYRSHsL |
| 821 | KIGsIIFQV | 1039 | KPQTRGKtF |
| 823 | KIIsIFSG | 1043 | KPRPLsMDL |
| 824 | KIIsIFSGTEK | 1047 | KPRPPPLsP |
| 825 | KIKsFEVVF | 1049 | KPRRFsRSL |
| 832 | KIRPHIAtL | 1051 | KPRsPDHVL |
| 852 | KLFPDtPLAL | 1054 | KPRsPFSKI |
| 866 | KLIDRTEsL | 1056 | KPRsPPRAL |
| 870 | KLKDRLPsI | 1062 | KPRsPPRALVL |
| 881 | KLLDFGsLSNLQV | 1073 | KPRsPVVEL |
| 898 | KLMsDVEDV | 1077 | KPSsPRGSL |
| 900 | KLMsPKADVKL | 1078 | KPSsPRGSLL |
| 902 | KLPDsPALA | 1080 | KPVsPKSGTL |
| 903 | KLPDsPALAK | 1083 | KPYsPLASL |
| 904 | KLPDsPALAKK | 1087 | KQKsLTNLSF |
| 908 | KLPsPAPARK | 1096 | KRAsFAKSV |
| 915 | KLRsPFLQK | 1112 | KRAsVFVKL |
| 921 | KLSGLsF | 1116 | KRAsYILRL |
| 932 | KLwtLVSEQTRV | 1119 | KRFsFKKSF |
| 937 | KLYTyIQSRF | 1120 | KRFsFKKsFKL |
| 943 | KMDsFLDMQL | 1132 | KRFsGTVRL |
| 980 | KMPTtPVKAK | 1136 | KRFsLDFNL |
| 1001 | KPAsPARRL | 1137 | KRIsIFLSM |
| 1006 | KPAsPKFIVTL | 1140 | KRKsFTSLY |
| 1011 | KPFKLSGLsF | 1142 | KRLEKSPsF |
| 1012 | KPGLGEGtP | 1145 | KRLsPAPQL |
| 1017 | KPPHsPLVL | 1151 | KRLsVELTSSLF |
| 1021 | KPPsPEHQSL | 1160 | KRMsNELENY |
| 1027 | KPPsPSPIEM | 1166 | KRMsPKPEL |
| 1029 | KPPtPGASF | 1175 | KRMsVTEGGIKY |
| 1183 | KRWQsPVTK | 1179 | KRsPIFF |
| 1190 | KRYsGNMEY | 1182 | KRTsKYFSL |
| 1191 | KRYsRALYL | 1275 | LESPTtPLL |
| 1195 | KSDsPSTSSI | 1277 | LIDNsFNRY |
| 1198 | KSKsMDLGI | 1280 | LLARtPPAA |
| 1206 | KSPTsPLNM | 1282 | LLDPSRSYsY |
| 1208 | KsSSLDKQL | 1290 | LLNKtPPTA |
| 1208 | KSsSLDKQL | 1291 | LMFsPVTSL |
| 1208 | KSSsLDKQL | 1297 | LPAFKRKtL |
| 1214 | KSYsRSRsR | 1299 | LPAsPAHQL |
| 1219 | KTFsIGKIAK | 1305 | LPAsPRARL |
| 1226 | KTMsGTFLL | 1309 | LPAsPSVSL |
| 1230 | KTPTsPLKM | 1214 | LPIFSRLsI |
| 1231 | KTPTsPLKMK | 1320 | LPKGLSAsL |
| 1234 | KTRsLSVEI | 1325 | LPLsPKETV |
| 1235 | KTRsLsVEIVY | 1329 | LPRGSsPSVL |
| 1235 | KTRsLSVEIVY | 1334 | LPRPAsPAL |
| 1238 | KTVsPSPAF | 1338 | LPRSSsMAA |
| 1240 | KVAsLLHQV | 1339 | LPRSSsMAAGL |
| 1241 | KVDsPTVTTTL | 1346 | LPSSGRSsL |
| 1242 | KVDsPVIF | 1348 | LPTsPLAMEY |
| 1247 | KVKSsPLIEKL | 1351 | LPYPVsPKQKY |
| 1252 | KVLsSLVTL | 1353 | LQIsPPLHQHL |
| 1252 | KVLSSLVTL | 1354 | LQIsPVSSY |
| 1252 | KVLssLVTL | 1359 | LSAsFRSLY |
| 1262 | KVQsLRRAL | 1365 | LSDsPSMGRY |
| 1265 | KVYtPSISK | 1374 | LSSsPPATHF |
| 1266 | KYELsVIM | 1377 | LTDPSsPTIS |
| 1270 | LADsPLKL | 1388 | LTLsPKLQL |
| 1274 | LEItPPSSEKL | 1389 | LTSsRLLKL |
| 1275 | LESPTtPLL | 1391 | LVAsPRLEK |
| 1275 | LESPttPLL | 1393 | LVVsPGQQTL |
| 1413 | MPsPGGRITLM | 1394 | LYTyIQSRF |
| | RLsRELQL | | |
| 1433 | MTRsPPRVSK | 1409 | MPRQPsATRL |
| 1436 | NAIsLPTI | 1563 | RAHsEPLAL |
| 1449 | NMDsPGPML | 1565 | RAHSsPASL |
| 1452 | NPsSPEFFM | 1566 | RAHtPTPGIYM |
| 1452 | NPSSPEFFM | 1567 | RAIsPREKI |
| 1452 | NPssPEFFM | 1569 | RAKsPISLK |
| 1458 | NRMsRRIVL | 1572 | RALsSSVIREL |
| 1472 | NSLsPRSSL | 1576 | RAPsPSSRM |
| 1484 | PLVSSSDsPPRPQPAF | 1578 | RARGIsPIVF |
| 1488 | PRFsLDAEIDSL | 1579 | RAsSDIVSL |
| 1489 | PRPANsGGVDL | 1579 | RASsDIVSL |
| 1490 | PRPsPGSNSKV | 1579 | RASSDIVsL |
| 1491 | PRPsPRQNSI | 1579 | RAssDIVSL |
| 1492 | PRQRAtSNVF | 1579 | RAsSDIVsL |
| 1494 | PRWsPAVSA | 1579 | RASsDIVsL |
| 1498 | PtSPLAMEY | 1579 | RAssDIVsL |
| 1498 | PTsPLAMEY | 1586 | RATsNVFAM |
| 1498 | PtsPLAMEY | 1587 | RATsPLVSL |
| 1499 | PVRdPTRSP | 1588 | RATsRcLQL |
| 1500 | PWIPPSsPTTF | 1589 | RAVsPFAKI |
| 1507 | QLDRIsVYY | 1590 | REAPsPLMI |
| 1508 | QLDsPQRALY | 1592 | REAsPAPLA |
| 1517 | QPRsPGPDYSL | 1593 | REAsPLSSNKLIL |
| 1527 | QPRtPSPLVL | 1594 | REAsPRLRV |
| 1527 | QPRtPsPLVL | 1595 | REAsPSRLSV |
| 1534 | QPSsPRVNGL | 1600 | REIMGtPEYL |
| 1536 | QRLsPLSAAY | 1602 | REKsPGRML |
| 1542 | QTIsPLSTY | 1606 | RELARKGsL |
| 1546 | QVDPKKRIsM | 1607 | RELsGTIKEIL |
| 1552 | RADsPVHM | 1608 | RELsPLISL |
| 1561 | RAFsVKFEV | 16611 | REPsPALGPNL |
| 1616 | RERsPSPSF | 1612 | REPsPLPEL |
| 1618 | REsPIPIEI | 1614 | REPsPVRYDNL |
| 1620 | RESSPTRRL | 1730 | RLFVGsIPK |
| 1621 | REtSPNRIGL | 1737 | RLIsQIVSSI |
| 1621 | RETsPNRIGL | 1743 | RLKsIEERQLLK |
| 1621 | REtsPNRIGL | 1755 | RLLDPSSPLAL |
| 1623 | REVsPAPAV | 1756 | RLLDRSPsRSAK |
| 1628 | REYGsTSSI | 1772 | RLLsPPLRPR |
| 1629 | RFKtQPVTF | 1773 | RLLsPQQPAL |
| 1630 | RFsFKKSF | 1779 | RLLsTDAEAV |
| 1631 | RGDGYGtF | 1800 | RLPtRLPEI |
| 1636 | RHPKRSVsL | 1814 | RLRSsLVFK |
| 1637 | RIDIsPSTL | 1821 | RLSDtPPLL |
| 1640 | RIHGsPLQK | 1825 | RLSsLRASTSK |
| 1642 | RILsGVVTK | 1827 | RLSsPISKR |
| 1650 | RIPsVQINF | 1829 | RLSsPLHFV |
| 1656 | RIStPLTGV | 1844 | RLYKsEPEL |
| 1657 | RITsLIVHV | 1910 | RMLsLRDQRL |
| 1659 | RIYQyIQ | 1948 | RMYsFDDVL |
| 1661 | RIYQyIQSR | 1966 | RNLsSPFIF |
| 1662 | RIYQyIQSRF | 1967 | RPAFFsPSL |
| 1664 | RIYQyIQSRFY | 1969 | RPAKsMDSL |
| 1672 | RKLsVILIL | 1974 | RPAsAGAML |
| 1675 | RKPsIVTKY | 1978 | RPAsARAQPGL |
| 1677 | RKSsIIIRM | 1983 | RPAsPAAKL |
| 1687 | RLAsLQSEV | 1987 | RPAsPEPEL |
| 1703 | RLAsYLDRV | 1988 | RPAsPGPSL |
| 1715 | RLDsYVRSL | 1989 | RPAsPLMHI |
| 1721 | RLFsHPREPAL | 1990 | RPAsPQRAQL |
| 1722 | RLFsKEL | 1991 | RPAsPSLQL |
| 1723 | RLFsKELR | 1992 | RPAsPSLQLL |
| 1725 | RLFsKELRC | 1996 | RPAsYKKKSML |
| 2012 | RPDsRLGKTEL | 1997 | RPAtFFPFVA |
| 2015 | RPDsRLLEL | 2009 | RPDsPTRPTL |
| 2016 | RPDVAKRLsL | 2135 | RPQRAtsNVF |
| 2025 | RPEsPAGPF | 2135 | RPQRATsNVF |
| 2026 | RPFHGISTVsL | 2141 | RPRAAtVV |
| 2028 | RPFsPREAL | 2142 | RPRAAtVVA |
| 2035 | RPGsRQAGL | 2144 | RPRANsGGVDL |
| 2037 | RPHsPEKAF | 2147 | RPRARsVDAL |
| 2041 | RPHtPTPGI | 2148 | RPRDTRRIsL |
| 2042 | RPHtPTPGIYM | 2150 | RPRGsESLL |
| 2047 | RPIsPGLSY | 2152 | RPRGsQSLL |
| 2048 | RPIsPPHTY | 2155 | RPRHsLNSL |
| 2049 | RPIsPRIGAL | 2156 | RPRIPsPIGF |
| 2050 | RPIsVIGGVSL | 2157 | RPRPAsSPAL |
| 2052 | RPItPPRNSA | 2158 | RPRPGtGLGRVm |
| 2057 | RPKLHHSLsF | 2160 | RPRPHsAPSL |
| 2059 | RPKLSSPAL | 2163 | RPRPSsAHVGL |
| 2064 | RPKPSSsPV | 2164 | RPRPsSVL |
| 2069 | RPKsNIVLL | 2164 | RPRPSsVL |
| 2073 | RPKsPLSKM | 2164 | RPRPssVL |
| 2079 | RPKsVDFDSL | 2168 | RPRPVsPSSL |
| 2082 | RPKtPPVVI | 2169 | RPRPVsPSSLL |
| 2089 | RPLSLLLAL | 2176 | RPRsAVEQL |
| 2106 | RPMsESPHM | 2178 | RPRsAVLL |
| 2108 | RPNsPSPTAL | 2181 | RPRSGsTGSSL |
| 2110 | RPPItQSSL | 2181 | RPRSGStGSSL |
| 2119 | RPPsPGPVL | 2181 | RPRSGstGSSL |
| 2127 | RPPsSEFLDL | 2183 | RPRsISVEEF |
| 2131 | RPPtPTLSL | 2183 | RPRSIsVEEF |
| 2131 | (diMe)RPPItQSSL | 2183 | RPRsIsVEEF |
| 2135 | RPQRAtSNVF | 2187 | RPRsLEVTI |
| 2135 | RPQRATsNVF | 2193 | RPRSLsSPTVTL |
| 2197 | RPRsMTVSA | 2194 | RPRSLSsPTVTL |
| 2198 | RPRsMVRSF | 2194 | RPRSLssPTVTL |
| 2200 | RPRsPAARL | 2275 | RPsSPALYF |
| 2204 | RPRsPGSNSKVP | 2275 | RPSsPALYF |
| 2205 | RPRsPNMQDL | 2275 | RPssPALYF |
| 2206 | RPRsPPGGP | 2282 | RPSsPSTSw |
| 2210 | RPRsPPPRAP | 2283 | RPSsRAVLY |
| 2212 | RPRsPPSSP | 2293 | RPsTPTIDVL |
| 2214 | RPRsPRENSI | 2293 | RPStPTIDVL |
| 2218 | RPRsPRPPP | 2293 | RPstPTIDVL |
| 2221 | RPRsPRQNSI | 2297 | RPTsFADEL |
| 2227 | RPRsPSPIS | 2298 | RPTsISwDGL |
| 2227 | RPRSPsPIS | 2299 | RPtSPIQIM |
| 2227 | RPRsPsPIS | 2299 | RPTsPIQIM |
| 2234 | RPRsPTGP | 2299 | RPtsPIQIM |
| 2235 | RPRsPTGPSNSF | 2302 | RPTsRLNRL |
| 2236 | RPRsPTGPSNSFL | 2310 | RPVsPFQEL |
| 2240 | RPRsPTGsNSF | 2314 | RPVsPGKDI |
| 2246 | RPRsPWGKL | 2319 | RPVsPHSDF |
| 2247 | RPRsQYNTKL | 2321 | RPVsPSAYm |
| 2250 | RPRtPLRSL | 2323 | RPVsPSSLL |
| 2255 | RPsLGGRTPL | 2324 | RPVsTDFAQY |
| 2258 | RPSRSsPGL | 2326 | RPVtPITNF |
| 2262 | RPsSAPDLM | 2330 | RPVtPVSDL |
| 2262 | RPSsAPDLM | 2334 | RPWsNSRGL |
| 2262 | RPssAPDLM | 2334 | RPwsNSRGL |
| 2263 | RPsSGFYEL | 2336 | RPWsPAVSA |
| 2263 | RPSsGFYEL | 2336 | RPwsPAVSA |
| 2263 | RPssGFYEL | 2345 | RPYsPPFFSL |
| 2270 | RPsSLPDL | 2348 | RPYsPSEYAL |
| 2270 | RPSsLPDL | 2350 | RPYsQVNVL |
| 2270 | RPssLPDL | 2352 | RQAsIELPSM |
| 2362 | RQDsTPGKVFL | 2353 | RQAsIELPSMAV |
| 2366 | RQIsQDVKL | 2356 | RQAsLSISV |
| 2371 | RQKsPLFQF | 2482 | RRGsGPEIF |
| 2374 | RQLsSGVSEI | 2483 | RRGsGPEIFT |
| 2378 | RQPsEEEII | 2485 | RRGsLLGSM |
| 2379 | RQPsEEEIIKL | 2489 | RRGsYPFIDF |
| 2383 | RQSsFEPEF | 2490 | RRHsASNLHAL |
| 2391 | RRAsIITKY | 2492 | RRIDIsPSTF |
| 2392 | RRAsLSEIGF | 2500 | RRIsDPEVF |
| 2395 | RRAsLSYSF | 2501 | RRIsDPQVF |
| 2409 | RRDsIVAEL | 2514 | RRIsIGSLF |
| 2412 | RRDsLQKPGL | 2516 | RRIsQIQQL |
| 2413 | RRFsDFLGLRR | 2517 | RRIsVFKYV |
| | FsGTAVY | | |
| 2433 | RRFsIATLR | 2534 | RRKsQVAEL |
| 2434 | RRFsLSPSL | 2544 | RRLsAARLL |
| 2435 | RRFsLTTLR | 2547 | RRLsADIRL |
| 2436 | RRFsLTTLRNF | 2552 | RRLsELLRY |
| 2437 | RRFsLTTLRNY | 2560 | RRLsFLVSY |
| 2446 | RRFsPPRRM | 2561 | RRLsFQAEY |
| 2451 | RRFsRSDEL | 3975 | RRLsFSTRL |
| 2455 | RRFSRsPIR | 2562 | RRLsGELISM |
| 2459 | RRFsRsPIRR | 2568 | RRLsGGSHSY |
| 2465 | RRFsSYSQM | 2575 | RRLsLFLNV |
| 2466 | RRFsTEYEL | 2576 | RRLsLFLVL |
| 2466 | RRFStEYEL | 2577 | RRLsLPGLL |
| 2466 | RRFstEYEL | 2578 | RRLsLSRSL |
| 2468 | RRFsVSTLRNL | 2604 | RRLsRKL |
| 2469 | RRFsVSTLRNLGL | 2605 | RRLsRKLSL |
| 2470 | RRFsVSTLRNLGLG | 2607 | RRLsVEIYDKF |
| 2471 | RRFsVSTLRNLGLGK | 2613 | RRLsYVLFI |
| 2479 | RRGsFEVTL | 2619 | RRLtLHSVF |
| 2480 | RRGsFEVTLL | 2620 | RRMsFQKP |
| 2639 | RRMsVAEQVDY | 2621 | RRMsFSGIFR |
| 2640 | RRMsVGDRAG | 2627 | RRMsLLSVV |
| 2641 | RRNsAPVSV | 2780 | RRVsSNGIFDL |
| 2642 | RRNsFIGTPY | 2783 | RRVVQRSsL |
| 2645 | RRNsKIFLDL | 2787 | RRYsASTVDVIEM |
| 2646 | RRNsLLHGY | 2801 | RRYsLPLKSIYM |
| 2672 | RRPsIAPVL | 2808 | RRYsPPIQR |
| 2674 | RRPsLLSEF | 2820 | RSAsFSRKV |
| 2681 | RRPsLVHGY | 2821 | RSAsLAKL |
| 2684 | RRPsQPYMF | 2825 | RSAsPSSQGw |
| 2695 | RRPsYRKIL | 2826 | RSAsPTVPR |
| 2702 | RRPsYTLGM | 2830 | RSAsVGAEEY |
| 2711 | RRRsLERLL | 2841 | RSDsYVEL |
| 3976 | RRRsRVFDL | 2845 | RSEsKDRKL |
| 2713 | RRSsDIISL | 2850 | RSEsPPAEL |
| 2714 | RRSsFLQ | 2853 | RSEsTENQSY |
| 2715 | RRSsFLQVF | 3977 | RSFsPKSPLEL |
| 2728 | RRSsIPITV | 2865 | RSGsLERKV |
| 2730 | RRsSIQSTF | 3978 | RSHsLHYLF |
| 2730 | RRSsIQSTF | 2866 | RSHsPLRSK |
| 2730 | RRssIQSTF | 2867 | RSHsPMSNR |
| 2740 | RRSsLDAEIDSL | 2875 | RSIsTPTcL |
| 2743 | RRSsLLSLM | 2877 | RSKsATLLY |
| 2746 | RRsSQSWSL | 3979 | RSKsSImYF |
| 2746 | RRSsQSwSL | 2879 | RSKtPPKSY |
| 2746 | RRSsQSWSL | 2880 | RSLGsVQAPSY |
| 2771 | RRsSYLLAI | 2881 | RSLsASPAL |
| 2771 | RRSsYLLAI | 2883 | RSLsESYEL |
| 2771 | RRssYLLAI | 2886 | RSLsPGGAA |
| 2777 | RRVsIGVQL | 2894 | RSLsPLLF |
| 2778 | RRVsPLNL | 2898 | RSLsPSSNSAF |
| 2779 | RRVsPLNLSSVTP | 2899 | RSLsQELVGV |
| 2901 | RSLSsGESL | 2901 | RSLsRVRVL |
| 2901 | RSLssGESL | 2901 | RSLsSGESL |
| 2903 | RSLsTTNVF | 2965 | RSVsPVQDL |
| 2905 | RSLsVEIVY | 3982 | RSYsRLETL |
| 2907 | RSLsVPVDL | 2983 | RSYsRSFSR |
| 2913 | RSNsLVSTF | 2985 | RSYsYPRQK |
| 2916 | RsPEPDPYLSY | 2988 | RTAsFAVRK |
| 2917 | RSPsFNMQL | 2992 | RTAsLVSGL |
| 2918 | RSPsKPTLAY | 2994 | RTAsPPPPPK |
| 2922 | RsPTKSSLDY | 2998 | RTDsIGEKLGRY |
| 3980 | RSRsDNALHL | 3004 | RTDsRGVNL |
| 2928 | RSRsPLGFY | 3008 | RTEsDSGLKK |
| 2933 | RSRsPRPAL | 3022 | RTFsPTYGL |
| 2936 | RSRsRDRMY | 3023 | RTFsPTYGLLR |
| 2939 | RSRsYTPEY | 3027 | RTGsPALGL |
| 2943 | RSSPRTIsF | 3028 | RTHsLLLLL |
| 2944 | RSSQFGsLEF | 3030 | RTIsNPEVVMK |
| 2945 | RSSsAPLGL | 3031 | RTIsPPTLGTL |
| 2947 | RSSsFSDTL | 3032 | RTIsQSSSL |
| 2948 | RsSSFVLPKL | 3033 | RtISVILFL |
| 2948 | RSSSFVLPKL | 3033 | RTIsVILFL |
| 2948 | RSSSFVLPKL | 3033 | RtIsVILFL |
| 2948 | RsSSFVLPKL | 3983 | RTLHsPPLQL |
| 2948 | RSssFVLPKL | 3034 | RTLsHISEA |
| 2948 | RsssFVLPKL | 3038 | RTLsPSSGY |
| 2948 | RSSSFVLPKL | 3042 | RTNsPGFQK |
| 2954 | RSSsPLQL | 3046 | RTPsISFHH |
| 2959 | RSVsGFLHF | 3051 | RTPsPKSLPSYL |
| 2960 | RSVsLDSQM | 3052 | RTRsLPITI |
| 2961 | RSVsLDSQMGY | 3055 | RTRsLSSLREK |
| 2964 | RSVsPTFL | 3058 | RTRsPSPTL |
| 3981 | RSVsPTTEM | 3058 | RTsSFALNL |
| 3062 | RTSsPLFNK | 3058 | RTSSFALNL |
| 3063 | RTSsQRSTLTY | 3062 | RTssFALNL |
| 3065 | RTVsPELIL | 3163 | RYRsPEPDPYLSY |
| 3070 | RTYsGPMNKV | 3167 | SAGGsAEALLSDLH |
| 3071 | RTYsHGTYR | 3168 | SAGGsAEALLSDLHAF |
| 3072 | RTYsLGSAL | 3169 | SAIsPKSSL |
| 3984 | RVAsPKLVm | 3171 | SAKsPLPSY |
| 3079 | RVAsPSRKV | 3172 | SAMsPTHHL |
| 3080 | RVAsPTSGV | 3175 | SAYGGLTsPGLS |
| 3081 | RVAsPTSGVK | 3178 | sDDEKMPDLE |
| 3087 | RVDsPSHGL | 3180 | SDMPRAHsF |
| 3100 | RVKtPTSQSYR | 3182 | SDsPPRPQPAF |
| 3104 | RVKVDGPRSPsY | 3186 | SEAsPSREAI |
| 3109 | RVLsPLIIK | 3193 | SELsPGRSV |
| 3114 | RVPsKsLDL | 3197 | SESKsMPVL |
| 3114 | RVPsKSLDL | 3201 | SFDsGIAGL |
| 3114 | RVPSKsLDL | 3202 | SFDsGSVRL |
| 3119 | RVRKLPsTTL | 3202 | SFDsGsVRL |
| 3121 | RVRQsPLATR | 3202 | SFDSGsVRL |
| 3123 | RVRRsSFLNAK | 3210 | sGPEIFTF |
| 3123 | RVRRSSFLNAK | 3214 | SIDsPQKL |
| 3123 | RVRRSSFLNAK | 3216 | SIGsPVKVGK |
| 3129 | RVRsPTRSP | 3217 | sIISPDFSF |
| 3141 | RVVPsPLQF | 3217 | sIIsPDFSF |
| 3144 | RVVsPGIDL | 3217 | SIIsPNFSF |
| 3146 | RVWEDRPsSA | 3221 | SIMsPEIQL |
| 3146 | RVWEDRPSsA | 3222 | SIPsGYLEL |
| 3146 | RVWEDRPssA | 3226 | SISsMEVNV |
| 3154 | RVYsPYNHR | 3227 | SISStPPAV |
| 3159 | RYLGGsMDLSTF | 3985 | SISVQVNSIKFDsE |
| 3160 | RYPsNLQLF | 3240 | SLDsPSYVLY |
| 3162 | RYQtQPVTL | 3243 | sLEEPKQANGGAY |
| 3249 | SLFsPQNTL | 3245 | SLFGGsVKL |
| 3255 | SLHDIQLsL | 3247 | SLFsGDEENA |
| 3274 | SLNSsPVSK | 3357 | SPRGSGsSTSL |
| 3275 | SLQPRSHsV | 3358 | SPRLPRsPRL |
| 3276 | SLQsLETSV | 3363 | SPRRsRSISL |
| 3280 | SLSsLLVKL | 3366 | SPRsESGGL |
| 3284 | SLYDRPAsYS | 3375 | SPRsPGKPM |
| | MSSLSREV | | |
| 3298 | SMTRsPPRV | 3379 | SPRsPGRSL |
| 3303 | SPAsPKISL | 3992 | sPRsPGRSL |
| 3307 | SPDHSDHtL | 3993 | SPRsPQLSDF |
| 3308 | SPDsSQSSL | 3385 | SPRsPSTTYL |
| 3308 | SPDSsQSSL | 3389 | SPRsPVPTTL |
| 3308 | SPDssQSSL | 3391 | SPRtPPQRF |
| 3312 | SPEKAGRRsSL | 3404 | SPSsPSVRRQL |
| 3318 | SPFKRQLsL | 3408 | SPSTSRSGGsSRL |
| 3321 | SPFLSKRsL | 3421 | SQILRTPsL |
| 3321 | SPFLsKRSL | 3426 | SRHsGPFFTF |
| 3321 | SPFLsKRsL | 3428 | SRLsLRRSL |
| 3986 | SPFQSsPLSL | 3445 | SRSSSVLsL |
| 3986 | SPFQSSPLsL | 3471 | SSDsPPRPQPAF |
| 3324 | SPGLARKRsL | 3476 | SSDsPTNHFF |
| 3987 | SPGsPLHSL | 3485 | SSGRsPSKAVAAR |
| 3988 | SPGsPLVSm | 3489 | SSMKsPLYL |
| 3989 | SPHtPSTHF | 3490 | SSMsPLPQM |
| 3332 | sPHYFSPFRPY | 3493 | SsPEFFM |
| 3334 | sPIKVTL | 3497 | SSSGsPHLY |
| 3336 | SPKSGsPKSSSL | 3799 | SSSSSGsPHLY |
| 3337 | SPKsPGLKA | 3501 | SsVPGVRLL |
| 3990 | SPPNLtPKPL | 3506 | SSYPRPLtY |
| 3991 | SPRDsPAVSL | 3511 | STDsETLRY |
| 335 | SPRERsPAL | 3526 | STIAILNsV |
| 3356 | SPRGEASsL | 3531 | STKsTELLL |
| 3994 | STsSGRLLY | 3532 | STLLAsPMLK |
| 3536 | SVDISPTRL | 3534 | STPsGYLEL |
| 3538 | SVFRHFGsFQK | 3616 | TPAQPQRRsL |
| 3545 | SVKPRRTsL | 3627 | TPIsPGRASGM |
| 3995 | SVKsPEVQLL | 3630 | TPIsPLKTGV |
| 3546 | SVKsPVTVK | 3632 | TPKsPGASNF |
| 3548 | SVLPRALSL | 3633 | TPMKKHLsL |
| 3553 | SVMQsPLVGV | 3634 | TPPPPPDtPP |
| 3556 | SVRRsVLMK | 3640 | TPRsPPLGL |
| 3561 | SVsSLEVHF | 3642 | TPRsPPLGLI |
| 3561 | SVSSLEVHF | 3651 | TPSsREGTL |
| 3561 | SVssLEVHF | 3653 | TPVSPRLHV |
| 3561 | SVSSLEVHF | 3654 | tPVSPTASM |
| 3568 | sYIEHIFEI | 3657 | TRDsLLIHL |
| 3572 | sYQKVIELF | 3658 | TRKTPEsFL |
| 3573 | SYSFSSSsIGH | 3658 | TRKtPESFL |
| 3574 | SYSYSFSSSsIGH | 3658 | TRKtPEsFL |
| 3578 | TAIsPPLSV | 3670 | TSDsPPHNDI |
| 3579 | TAPLVPPLsPQY | 3674 | TsFADEL |
| 3580 | TASPVAVsL | 3676 | TSGPGSRISSSSF |
| 3586 | TEPLPEKTQEsL | 3678 | TSIsPSRHGAL |
| 3587 | TESsPGSRQIQLW | 3696 | TVFsPTLPAA |
| 3589 | THsLLLLL | 3703 | TVYsSEEAELLK |
| 3591 | TIRsPTTVL | 3703 | TVYSsEEAELLK |
| 3996 | TKSsPLKI | 3703 | TVYssEEAELLK |
| 3598 | TLAsPSVFKST | 3704 | TYEGIFKtL |
| 3600 | TLDsLDFARY | 3708 | VAKRLsL |
| 3602 | TLLAsPMLK | 3711 | VEKLPDsPAL |
| 3603 | TLLsPSSIKV | 3713 | VELsPARSW |
| 3604 | TLMERTVsL | 3720 | VLDsPASKK |
| 3607 | TMAsPGKDNY | 3723 | VLFSSPPQM |
| 3614 | TPAPSRTAsF | 3725 | VLIENVAsL |
| 3997 | VLVVDTPsI | 3734 | VLLsPVPEL |
| 3744 | VLYsPQMAL | 3741 | VLSDVIPsI |
| 3745 | VMDsPVHL | 3835 | YEGsPIKVTL |
| 3746 | VMFPGNSPSY | 3838 | YFsPFRPY |
| 3747 | VMFRtPLASV | 3840 | YGITsPISL |
| 3748 | VMIGsPKKV | 3841 | YHLSPRAFLHY |
| 3774 | VPRPERRSsL | 3843 | yIQSRF |
| 3774 | VPRPERRssL | 3858 | yLQSRYYRA |
| 3774 | VPRPERRsSL | 3865 | YPGGRRsSL |
| 3998 | VPRPStPSRL | 3868 | YPLsPAKVNQY |
| 3776 | VPRsPKHAHSSSL | 3869 | YPLsPTKISEY |
| 3784 | VPTsPKSSL | 3670 | YPLsPTKISQY |
| 3788 | VPVsPGQQL | 3889 | YQRPFsPSAY |
| 3801 | VsPFQEL | 3893 | YQRsFDEVEGV |
| 3803 | VSsPPPYTAY | 3894 | YQRsFDEVEGVF |
| 3804 | VSSSDsPPRPQPAF | 3900 | YRYsPQSFL |
| 3807 | VTQtPPYVKK | 3901 | YSDRsSGGSY |
| 3808 | VTtPNRLIY | 3902 | YSEsRSSLDY |
| | | | YSFsPSKSY |
| 3809 | VTtPTGYKY | 3905 | YSFSSSsIGH |
| 3811 | VVDsPGQEVL | 3909 | YSLsPRPSY |
| 3814 | VYIPMSPGAHHF | 3915 | YTDSESSAsL |
| 3815 | VYLPTHTsL | 3916 | YTsSRDAFGY |
| 3820 | VYTyIQSRF | 3916 | YTSsRDAFGY |
| 3999 | yAQPQTTTPLPAVSG | 3916 | YTSSRDAFGY |
| 3831 | YEFsPVKML | 3917 | YVDAETsL |
| 3832 | YEGsPIKV | 3918 | YVKLTPVsL |
| 3833 | YEGsPIKVT | 3919 | YVPDsPALL |
| | | 3920 | YVSsPDPQL |
| | | 4000 | yYPDPHsPFAV |

TABLE 7
Peptides of the Presently Disclosed
Subject Matter

| | |
|---|---|
| 1. | AAAsPLHML |
| 2. | AADGtPKHSF |
| 3. | AADsPSQNL |
| 4. | AADsPSQNLT |
| 5. | AADtPPLETL |
| 6. | AAEsPSFL |
| 7. | (AcS)AARESHPHGVKRSAsP |
| | DDDLG |
| 8. | AAsDTERDGLA |
| 9. | AASNFKsPVKTIR |
| 10. | AAsPGAPQM |
| 11. | ADLsPEREV |
| 12. | ADsGEGDFLAEGGGVR |
| 13. | AEAPLPsPKL |
| 14. | AEAPPSKsP |
| 15. | AEDEIGtPRKF |
| 16. | AEDEIGtPRKY |
| 17. | AEEEIGtPRKF |
| 18. | AEEEIGtPRKW |
| 19. | AEEEIGtPRKY |
| 20. | AEFPSSGsNSVL |
| 21. | AEGsPPPKTY |
| 22. | AEIsPGSLP |
| 23. | AEIsPGSLPVTA |
| 24. | AEKsYQNSP |
| 25. | AELsPKNLL |
| 26. | AELsPSMAP |
| 27. | AELsPTTLSP |
| 28. | AELsPVEQKL |
| 29. | AEMPTQMsP |
| 30. | AENARSAsF |
| 31. | AENsPTRQQF |
| 32. | AENsPTRQQW |
| 33. | AENsPTRQQY |
| 34. | AENsSSREL |
| 35. | AEPtPEKEKRF |
| 36. | AEQGsPRVSY |
| 37. | AERtPELVEL |
| 38. | AEsPERVLL |
| 39. | AESsPTAGKKF |
| 40. | AESsPTAGKKL |
| 41. | AESsPTAGKKW |
| 42. | AESsPTAGKKY |
| 43. | AFsPVRSV |
| 44. | AGDsPGSQF |
| 45. | AILsPAFKV |
| 46. | AIMRsPQMV |
| 47. | AIsDLQQL |
| 48. | AKLsETIS |
| 49. | ALAAPsPPR |
| 50. | ALAAsPHAV |
| 51. | ALDsGASLLHL |
| 52. | ALDsGASLLHV |
| 53. | ALDsPPPPTL |
| 54. | ALDsQVPKV |
| 55. | ALGNtPPFL |
| 56. | ALGsRESLATI |
| 57. | ALGsRESLATL |
| 58. | ALGsRESLATV |
| 59. | ALIHQsLGL |
| 60. | ALIHQsLGV |
| 61. | ALLDIIRsL |
| 62. | ALLGSKsPDPYRL |
| 63. | ALLGSKsPDPYRV |
| 64. | ALLsLLKRV |
| 65. | ALMGsPQLV |
| 66. | ALMGsPQLVAA |
| 67. | ALRSSPIMRK |
| 68. | ALRSsPIMRY |
| 69. | ALSSLIHAL |
| 70. | ALStPVVEK |
| 71. | ALTsELANA |
| 72. | ALTtSAHSV |
| 73. | ALTTsAHSV |
| 74. | ALTtsAHSV |
| 75. | ALVSPPALHNA |
| 76. | ALVSPPALHNV |
| 77. | ALYsGVHKK |
| 78. | ALYsGVHKY |
| 79. | ALYsPAQPSL |
| 80. | ALYsPAQPSV |
| 81. | ALYtPQAPK |
| 82. | ALYtPQAPY |
| 83. | AMAAsPHAV |
| 84. | AMDsGASLLHL |
| 85. | AMDsGASLLHV |
| 86. | AMDsPLLKY |
| 87. | AMGAGHFsV |
| 88. | AMGsRESLATI |
| 89. | AMGsRESLATV |
| 90. | AMLGSKsPDPYRL |
| 91. | AMLGSKsPDPYRV |
| 92. | AMPGsPVEV |
| 93. | AMRSSPIMRK |
| 94. | AMVSPPALHNA |
| 95. | AMVSPPALHNV |
| 96. | AMYsGVHKK |
| 97. | APAGGsPRML |
| 98. | APAsPFRQL |
| 99. | APAsPFRQLL |
| 100. | APAsPLRPL |
| 101. | APAsPNHAGVL |
| 102. | APAsPRLL |
| 103. | APAsPTHPGL |
| 104. | APAsPTHPGLM |
| 105. | APDsPRAFL |
| 106. | APDsPSKQL |
| 107. | APGPGFSSRsL |
| 108. | APKsPSQDVKA |
| 109. | APLARASsL |
| 110. | APPAYEKLs |
| 111. | APPAYEKLsAEQ |
| 112. | APPAYEKLsAEQSPP |
| 113. | APPAYEKLsAEQSPPP |
| 114. | APPAYEKLsAEQSPPPY |
| 115. | APPPLVPAPRPSsPPRGPGPA |
| | RADR |
| 116. | APPsTSAAAL; |
| | APPStSAAAL; |
| | APPSTsAAAL; APPstSAAAL; |
| | APPStsAAAL; APPsTsAAAL |
| 117. | APRAPSASPLAL |
| 118. | APRDRRAVsF |
| 119. | APRGnVTSL |
| 120. | APRKGsFSAL |
| 121. | APRKGsFSALF |
| 122. | APRKGsFSALL |
| 123. | APRKGsFSALM |
| 124. | APRKGsFSALV |
| 125. | APRNGsGVAL |
| 126. | APRRYsSSF |
| 127. | APRRYsSSL |
| 128. | APRRYsSSM |
| 129. | APRRYsSSV |
| 130. | APRSPPPSRF |
| 131. | APRSPPPSRL |
| 132. | APRSPPPSRM |
| 133. | APRsPPPSRP |
| 134. | APRsPPPSRV |
| 135. | APRINGVAM |
| 136. | APRtPPGVTF |
| 137. | APSLFHLNtL |
| 138. | APSRQIsL |
| 139. | APSSARAsPLL |
| 140. | APSTYAHLsPAK |
| 141. | APSTYAHLsPAKTPPPP |
| 142. | APSVRSLsL |
| 143. | APSVRsLSL |
| 144. | APTsAAAL |
| 145. | APTsASNVM |
| 146. | APTSAsNVM |
| 147. | APVsASASV |
| 148. | APVsPLKF |
| 149. | APVsPRPGL |
| 150. | APVsPSSQKL |
| 151. | APVsSKSSL |
| 152. | APYRGQLAsPSSQ |
| 153. | AQDsPTHL |
| 154. | ARAsPRLHFL |
| 155. | ARFsGFYSM; ARFSGFYsM |
| 156. | ARFsPDDKYSF |
| 157. | ARFsPDDKYSK |
| 158. | ARFSPDDKYSL |
| 159. | ARFSPDDKYSM |
| 160. | ARFSPDDKYSR |
| 161. | ARFsPDDKYSY |
| 162. | ARFsPKVSL |
| 163. | ARGsLRRLL |
| 164. | ARIsRSISL |
| 165. | ARIsRSIsL |
| 166. | ARtsPINLGL |
| 167. | ARVsPSTSY |
| 168. | ASDEIGtPRKF |
| 169. | ASDEIGTPRKY |
| 170. | ASEsPSSLIFY; |
| | ASESPsSLIFY; |
| | ASESPSsLIFY; |
| | ASEsPsSLIFY; ASEsPSsLIFY; |
| | ASESPssLIFY |
| 171. | ASEsPssLIFY |
| 172. | ASEEIGtPRKF |
| 173. | ASEEIGTPRKY |
| 174. | ASEsPssLIFY |
| 175. | asGVAVSDGVIK |
| 176. | ASISRLsGEQVDGKG; |
| | AsISRLSGEQVDGKG; |
| | AsISRLsGEQVDGKG; |
| | AsIsRLSGEQVDGKGQ |
| 177. | ASKAsPTLDFTER |
| 178. | ASKMTQPQSKSAFPLSRKN |
| | KGsGsLDG |
| 179. | AsLGFVF |
| 180. | ASLsPSVSK |
| 181. | ASLsRPLNY |
| 182. | ASMsPGHPTHL |
| 183. | AsPTIEAQGTSPAHDN |
| 184. | AsPTIEAQGTSPAHDNI |
| 185. | AsPTIEAQGTSPAHDNIA |
| 186. | ASsPPDRIDIF |
| 187. | ASSsPVTLR |
| 188. | ASSsQIIHI |
| 189. | AtAGPRLGF |
| 190. | AtAGPRLGW; AtAGPRLGw |
| 191. | AtAGPRLGY |
| 192. | ATDEIGtPRKF |
| 193. | ATDEIGTPRKY |
| 194. | ATEEIGtPRKF |
| 195. | ATEEIGtPRKY |
| 196. | ATIPRPFsV |
| 197. | ATMKRMLsL |
| 198. | ATWsGSEFEV |
| 199. | ATYtPQAPK |
| 200. | ATYtPQAPKY |
| 201. | AVIHQsLGL |
| 202. | AVILPPLsPYFK |
| 203. | AVRPTRLsL |
| 204. | AVVsPPALHNA |
| 205. | AVVsPPALHNV |
| 206. | AyAQPQTTTPLPAVSG |
| 207. | AYEKLsAEQSPP |
| 208. | AYGGLTsPGLSY |
| 209. | DAKKsPLAL |
| 210. | DDDWTHLSSKEVDP |
| 211. | DDDWTHLsSKEVDPS |
| 212. | DDDWTHLsSKEVDPST |
| 213. | DDDWTHLsSKEVDPSTG |
| 214. | DDWTHLsSKEVDPS |
| 215. | DEFERIKtF |
| 216. | DEFERIKtW |
| 217. | DEFERIKtY |
| 218. | DEGPGHHHKPGLGEGtP |
| 219. | DEISHRAsF |
| 220. | DEISHRAsW |
| 221. | DEISHRAsY |
| 222. | DERLRINs |
| 223. | DERLRINsF |
| 224. | DERLRINsL |
| 225. | DERLRINsW |
| 226. | DERLRINsY |
| 227. | DETERAYsF |
| 228. | DGRRtFPRI |
| 229. | DKLsVIAEDSESGKQ |
| 230. | DKLsVIAEDSESGKQN |
| 231. | DKLsVIAEDSESGKQNP |
| 232. | DKLsVIAEDSESGKQNPG |
| 233. | DKLsVIAEDSESGKQNPGDS |
| 234. | DLKRRsMSI |
| 235. | DLKSSKAsL |
| 236. | DLRQAHsL |
| 237. | DLRtVEKEL |
| 238. | DLsEEKFL |
| 239. | DLsEEKFV |
| 240. | DLVPLsPLKK |
| 241. | DLWKItKVMD |
| 242. | DMVPLsPLKK |
| 243. | DPLSVsPARW |
| 244. | DPTRRFFKVtPPPGSGPQ |
| 245. | DQFERIKtL |
| 246. | DQISHRAsL |
| 247. | DRKsPRVL |
| 248. | DRKsPSVSL |
| 249. | DRLGsRPSL |
| 250. | DRQRsPIAL |
| 251. | DRSSPPtTPL |
| 252. | DRSSPPTtPL |
| 253. | DRSSPPttPL |
| 254. | DSDPLsPLKY |
| 255. | DsFESIESY |
| 256. | DSLARILsF |
| 257. | DssEEK |
| 258. | DSsEEKF; DssEEKF |
| 259. | DSsEEKFL; DssEEKFL |
| 260. | DSsEEKFLR; DssEEKFLR |
| 261. | DSsEEKFV |
| 262. | DSVPLsPLKY |
| 263. | DSVsPSESL |
| 264. | DTDPLsPLKY |
| 265. | DTDsAIGSFRY |
| 266. | DTEPLsPLKY |
| 267. | DTIsPTLGF |
| 268. | DTVPLsPLKY |
| 269. | DVYSGtPTKV |
| 270. | DWTHLsSKEVDPS |
| 271. | DWTHLsSKEVDPSTG |
| 272. | DyMDGTMSQV; |
| | DYMDGtMSQV; |
| | DYMDGTMsQV; |
| | DyMDGtMSQV; |
| | DyMDGTMsQV; |
| | DYMDGtMsQV; |
| | DyMDGtMsQV |
| 273. | DYSPYFKtI |
| 274. | EASsVTREL |
| 275. | EEAPQtPVAF |
| 276. | EEFsPRQAQMF |
| 277. | EEGsPTMVEKGLEPGVFTL |
| 278. | EEIGtPRKF |
| 279. | EELsPLALGRF |
| 280. | EELsPTAKF |
| 281. | EEMPENALPsDEDDKDPND |
| | PYRAL |
| 282. | EEQsFLQKF |
| 283. | EERRsPPAP |
| 284. | EERsPSWISA |
| 285. | EEsSDDGKKF; |
| | EESsDDGKKF |
| 286. | EEsSDDGKKW; |
| | EESsDDGKKW |
| 287. | EEsSDDGKKY; |
| | EESsDDGKKY |
| 288. | EGEEPTVYsDEEEPKDESAR |
| | KND |
| 289. | EGsPTMVEKGLEPGVFTL |
| 290. | EHVPSSSsI |
| 291. | EILNRsPRNR |
| 292. | ELFSsPPAV |
| 293. | ELKKsPTSLK |
| 294. | ELKKsPTSLY |
| 295. | ELLMPHRISSHF |
| 296. | ELLMPHRISSHFL |
| 297. | ELLPRRNsL |
| 298. | ELRISGsVQL |
| 299. | EMKKsPTSLK |
| 300. | EPAsPAAsISRLsGEQVDGKG |
| 301. | EPKRRsARF |
| 302. | EPKRRsARL |
| 303. | EPKRRsARM |
| 304. | EPKRRsARV |
| 305. | EPRNSLPAsPAHQL |
| 306. | EPRsPSHSF |
| 307. | EPRsPSHSL |
| 308. | EPRsPSHSM |
| 309. | EPRsPSHSV |
| 310. | EPsSTVVSL; EPSsTVVSL; |
| | EPSStVVSL |
| 311. | ERLKIRGsL |
| 312. | ERsPLLSQETAGQKP |
| 313. | ERsPLLSQETAGQKPL |
| 314. | ERVDSLVsL |
| 315. | ESDsLPRY |
| 316. | ESEsLPRY |
| 317. | ESsVRSQEDQLSR |
| 318. | ESsVRSQEDQLSRR |
| 319. | ETDsLPRY |
| 320. | ETEsLPRY |
| 321. | FAFPGStNSL; FAFPGSTNsL |
| 322. | FAFPGStNsL |
| 323. | FASPTsPPVL |
| 324. | FATIRTAsL |
| 325. | FAVsPIPGRGGVL |
| 326. | FAYGSGNsL |
| 327. | FAYsPGGAHGM |
| 328. | FAYsPGGAHGML |
| 329. | FDKHTLGDsDNES |
| 330. | FEDDDsNEKL |
| 331. | FGINsPQAL |
| 332. | FGLARAFsL |
| 333. | FGRKPsL |
| 334. | FGYDsPHDL |
| 335. | FIEsPSKL |
| 336. | FIEsPSKY |
| 337. | FIGsPTTPAGL |
| 338. | FKLSGLsF |
| 339. | FKMPQEKsPGYS |
| 340. | FKsPVKTIR |
| 341. | FKtQPVTF |
| 342. | FLDNsFEKV |
| 343. | FLDRPPtPLFI |
| 344. | FLDsAYFRL |
| 345. | FLDsLRDLI |
| 346. | FLDtPIAKV |
| 347. | FLFDKPVsPLLL |
| 348. | FLGVRPKsA |
| 349. | FLIIRtVLQL |
| 350. | FLITGGGKGsGFSL |
| 351. | FLLsPSDQEM |
| 352. | FLLsQNFDDE |
| 353. | FLMsDRSLHL |
| 354. | FLPSPDYFPSV |
| 355. | FLsRSIPSL |
| 356. | FLYsGKETK |
| 357. | FLYsGKETY |
| 358. | FPAsPSVSL |
| 359. | FPHsLLSVF |
| 360. | FPHsLLSVI |
| 361. | FPHsLLSVL |
| 362. | FPHsLLSVM |
| 363. | FPHsLLSVV |
| 364. | FPIsPVRF |
| 365. | FPIsPVRL |
| 366. | FPIsPVRM |
| 367. | FPIsPVRV |
| 368. | FPLARQFsL |
| 369. | FPLDsPKTLVL |
| 370. | FPLsPLRKY |
| 371. | FPLsPTKLSQY |
| 372. | FPRAsPRAL |
| 373. | FPRRHsVTL |
| 374. | FPRsPTKSSF |
| 375. | FPRsPTKSSL |
| 376. | FPRsPTKSSLDF |
| 377. | FPRsPTKSSLDL |
| 378. | FPRsPTKSSLDM |
| 379. | FPRsPTKSSLDV |
| 380. | FPRsPTKSSM |
| 381. | FPRsPTKSSV |
| 382. | FQYSKSPsL |
| 383. | FRFPsGAEL |
| 384. | FRFsGRTEY |
| 385. | FRGRYRsPY |
| 386. | FRKsMVEHY |
| 387. | FRRsPTKSSF |
| 388. | FRRsPTKSSL; FRRSPTKSSL |
| 389. | FRRsPTKSSLD |
| 390. | FRRsPTKSSLDF |
| 391. | FRRsPTKSSLDL |
| 392. | FRRsPTKSSLDM |
| 393. | FRRsPTKSSLD V |
| 394. | FRRsPTKSSLD Y |
| 395. | FRRsPTKSSM |
| 396. | FRRsPTKSSV |
| 397. | FRYsGKTEF |
| 398. | FRYsGKTEK |
| 399. | FRYSGKTEL |
| 400. | FRYSGKTEM |
| 401. | FRYsGKTER |
| 402. | FRYsGKTEY |
| 403. | FRYsGRTQA |
| 404. | FSDsHEGFSY |
| 405. | FSEsHEGFSY |
| 406. | FSEsPSKLw |
| 407. | FSEsPSKY |
| 408. | FsFAGFPSA |
| 409. | FsFKKSF |
| 410. | FSFKKsFKL |
| 411. | FsFKKSFKLS |
| 412. | FSIsPVRF |
| 413. | FSIsPVRL |
| 414. | FSIsPVRM |
| 415. | FSIsPVRV |
| 416. | FsSSHEGFSY; FSsSHEGFSY; |
| | FSSsHEGFSY; FssSHEGFSY; |
| | FsSsHEGFSY; FSSSHEGFSY |
| 417. | FSSsHEGFSY |
| 418. | FSVAsPLTL |
| 419. | FSVsPASTL |
| 420. | FSYsPRLPL |
| 421. | FTDsHEGFSY |
| 422. | FTDVNsILRY |
| 423. | FTEsHEGFSY |
| 424. | FTEsPSKL |
| 425. | FTEsPSKY |
| 426. | FTKsPYQEF |
| 427. | FTsSHEGFSY |
| 428. | FVsEGDGGRL |
| 429. | FVSKVMIGSPKKV |
| 430. | FVTtPTAEL |
| 431. | GALsPSLLHSL |
| 432. | GAQPGRHsF |
| 433. | GAQPGRHsL |
| 434. | GAQPGRHsV |
| 435. | GARTPSPsL |
| 436. | GATTTAPsL |
| 437. | GAVsPVGEL |
| 438. | GDDDWTHLSSKEVD |
| 439. | GDDDWTHLSSKEVDP |
| 440. | GDDDWTHLSSKEVDPS |
| 441. | GDDDWTHLSSKEVDPST |
| 442. | GDDDWTHLSSKEVDPSTG |
| 443. | GDEGPGHHHKPGLGEGtP |
| 444. | GEAsPLSSL |
| 445. | GEAsPSHII |
| 446. | GEEsSDDGKKF |
| 447. | GEEsSDDGKKW |
| 448. | GEEsSDDGKKY; |
| | GEEsSDDGKKY |
| 449. | GEFGGFGsV |
| 450. | GEIsPQREV |
| 451. | GEIsPTQIL |
| 452. | GEKsPPYGVP |
| 453. | GELPsPGKV |
| 454. | GELPTsPLHLL |
| 455. | GEMsPQRFFF |
| 456. | GENsGIGKLF |
| 457. | GEPHsSPEL |
| 458. | GEQsPNVSL |
| 459. | GERsPLLSQETAGQKP |
| 460. | GERsPLLSQETAGQKPL |
| 461. | GERsPPRIL |
| 462. | GETsLMRTL |
| 463. | GETsPHTFQL |
| 464. | GETsPRTKI |
| 465. | GETsPRTKITW |
| 466. | GETsYIRVY |
| 467. | GGDDDWTHLsSKEVDPS |
| 468. | GGDDDWTHLsSKEVDPSTG |
| 469. | GGDsPVRL |
| 470. | GGLTsPGLSY |
| 471. | GGPHFsPEHKEL |
| 472. | GGSFGGRSSGsP |
| 473. | GGSFGGRSSGsV |
| 474. | GHGsPFPSL |
| 475. | GHHHKPGLGEGtP |
| 476. | GHSKtILcM |
| 477. | GIDsPSSSV |
| 478. | GIFPGtPLKK |
| 479. | GIMsPLAKK |
| 480. | GKGGSYSQAASSDsAQG |
| 481. | GLAPNtPGKA |
| 482. | GLAPtPPSM |
| 483. | GLAsPTAITPV |
| 484. | GLDsGFHSV |
| 485. | GLDsLDQVEI |
| 486. | GLGELLRsL |
| 487. | GLIRSRsFIFK |
| 488. | GLIRSRsFIFY |
| 489. | GLIsPELRHL |
| 490. | GLIsPNVQL |
| 491. | GLIsPVWGA |
| 492. | GLItPGGFSSV |
| 493. | GLLDsPTSI |
| 494. | GLLGsPARL |
| 495. | GLLGsPVRA |
| 496. | GLLsPARLYAI |
| 497. | GLLsPARLYAV |
| 498. | GLLsPRFVDV |
| 499. | GLLsPRHSL |
| 500. | GLSFGGRSSGsP |
| 501. | GLSFGGRSSGsV |
| 502. | GLSsLAEEAA |
| 503. | GLSsLSIHL |
| 504. | GLTsPGLSY |
| 505. | GLTsPGLSYS |
| 506. | GLTsPGLSYSL |
| 507. | GMLGsPVRV |
| 508. | GMLsPARLYAI |
| 509. | GMLsPARLYAV |
| 510. | GMLsPGKSIEV |
| 511. | GPGHHHKPGLGEGtP |
| 512. | GPKPLFRRMsS |
| 513. | GPKPLFRRMSSL |
| 514. | GPKPLFRRMSSLV |
| 515. | GPKPLFRRMsSL VG |
| 516. | GPKPLFRRMSSL VGP |
| 517. | GPKPLFRRMsSL VGPT |
| 518. | GPKPLFRRMsSL VGPTQ |
| 519. | GPKPLFRRMsSL VGPTQS |
| 520. | GPLSRVKsL |
| 521. | GPLVRQIsL |
| 522. | GPPYQRRGsL |
| 523. | GPQPGRHsF |
| 524. | GPQPGRHsL |
| 525. | GPQPGRHsV |
| 526. | GPRAPsPTKPL |
| 527. | GPRASsLLsL |
| 528. | GPRPGsPSAF |
| 529. | GPRPGsPSAL |
| 530. | GPRPGsPSALL |
| 531. | GPRPGsPSAM |
| 532. | GPRPGsPSAV |
| 533. | GPRSAsLL |
| 534. | GPRSAsLLsF; GPRSASLLsF; |
| | GPRsASLLSF |
| 535. | GPRSASLLsL; GPRSAsLLSL; |
| | GPRSAsLLsL; GPRsAsLLSL; |
| | GPRsASLLSL |
| 536. | GPRSAsLLsM; GPRsASLLSM |
| 537. | GPRSAsLLsV; GPRSASLLsV; |
| | GPRsASLLSV |
| 538. | GPRsPKAPP |
| 539. | GPRsPPVTL |
| 540. | GPsSPWTQL; GPSsPWTQL; |
| | GPssPWTQL |
| 541. | GQLsPGVQF |
| 542. | GRKsPPPSF |
| 543. | GRKsPPPSK |
| 544. | GRKSPPPSL |
| 545. | GRKSPPPSM |
| 546. | GRKsPPPSR |
| 547. | GRKsPPPSY |
| 548. | GRLGsPHRF |
| 549. | GRLGsPHRK |
| 550. | GRLGSPHRL |
| 551. | GRLGSPHRM |
| 552. | GRLGsPHRR |
| 553. | GRLGsPHRY |
| 554. | GRLsPAYSL |
| 555. | GRLsPKASQVF |
| 556. | GRLsPKASQVK |
| 557. | GRLSPKASQVL |
| 558. | GRLSPKASQVM |
| 559. | GRLsPKASQVR |
| 560. | GRLsPKASQVY |
| 561. | GRLsPVPVPF |
| 562. | GRLsPVPVPK |
| 563. | GRLSPVPVPL |
| 564. | GRLSPVPVPM |
| 565. | GRLSPVPVPR |
| 566. | GRLSPVPVPY |
| 567. | GRQsPSFKL |
| 568. | GRsSPPPGY |
| 569. | GRSsTASLVKF |
| 570. | GRSSTASLVKK |
| 571. | GRSsTASLVKKK |
| 572. | GRSSTASLVKL |
| 573. | GRSSTASLVKM |
| 574. | GRSSTASLVKR |
| 575. | GRSsTASLVKR |
| 576. | GRSsTASLVKY |
| 577. | GSALGGGGAGLSGRASGGA |
| | QsPLRYLHV |
| 578. | GSDsPRSSL |
| 579. | GSDsSDDGKKY |
| 580. | GSDVsLTAcKV |
| 581. | GSEsSDDGKKY |
| 582. | GsGPEIFTF |
| 583. | GSKsPISQL |
| 584. | GsPHYFSPF |
| 585. | GSPHYFSPFRP |
| 586. | GSPHYFSPFRPY |
| 587. | GsPIKVTL |
| 588. | GsPIKVTLA |
| 589. | GsPTMVEKGLEPGVFTL |
| 590. | GsQLAVMMYL |
| 591. | GTDsSDDGKKY |
| 592. | GTEsSDDGKKY |
| 593. | GTFPKALsI |
| 594. | GTIRSRsFIFK |
| 595. | GTIRSRsFIFY |
| 596. | GTIsPTSSL |
| 597. | GtLPKY |
| 598. | GtLRRSDSQQAVK |
| 599. | GtLRRSDSQQAVKS |
| 600. | GtLRRSDSQQAVKSPP |
| 601. | GtPLSQAIIHQY |
| 602. | GTVtPALKL |
| 603. | GTYVPSSPTRLAY |
| 604. | GVAsPTITV |
| 605. | GVIsPQELLK |
| 606. | GVIsPQELLKK |
| 607. | GVIsPRFDVQL |
| 608. | GVVsPTFEL |
| 609. | GYVQRNLSLVRG |
| 610. | HAVsPIAKY |
| 611. | HEFMsDTNL |
| 612. | HEKKAYsF |
| 613. | HERGsLASL |
| 614. | HHHKPGLGEGtP |
| 615. | HHKPGLGEGtP |
| 616. | HIPsPAKKV |
| 617. | HKGEIRGASTPFQFRAssP |
| 618. | HKPGLGEGtP |
| 619. | HLHsPQHKL |
| 620. | HLYtSLPSL; HLYTsLPSL; |
| | HLYtsLPSL |
| 621. | HPFHAtPNTY |
| 622. | HPKRSVsL |
| 623. | HPLtPLITY |
| 624. | HPMsTASQV |
| 625. | HPRPTsQDL |
| 626. | HPRsPNVL |
| 627. | HPRsPNVLSF |
| 628. | HPRsPNVLSL |
| 629. | HPRsPNVLSM |
| 630. | HPRsPNVLSV |
| 631. | HPRsPTPTF; HPRSPtPTF |
| 632. | HPRsPTPTL; HPRSPtPTL |
| 633. | HPRsPTPTM; HPRSPtPTM |
| 634. | HPRSPtPTV; HPsSPTPTV |
| 635. | HPsSSAAVL; HPSsSAAVL; |
| | HPSSsAAVL; HPSstAAVL; |
| | HPssTAAVL |
| 636. | HPsSTASTAL; HPSsTASTAL; |
| | HPSStASTAL |
| 637. | HPTtVASY |
| 638. | HPYsPLPGL |
| 639. | HQGKFLQtF |
| 640. | HRFsINGHFY |
| 641. | HRLsPVKGEF |
| 642. | HRLsPVKGEK |
| 643. | HRLsPVKGER |
| 644. | HRLsPVKGEY |
| 645. | HRNsMKVFL |
| 646. | HRNsNPVIAEF |
| 647. | HRNsNPVIAEK |
| 648. | HRNsNPVIAEL |
| 649. | HRNSNPVIAEM |
| 650. | HRNsNPVIAER |
| 651. | HRNsNPVIAEY |
| 652. | HRVsVILKL |
| 653. | HRYsTPHAF; HRYStPHAF; |
| | HRYstPHAF |
| 654. | HRYPTSIASLAF |
| 655. | HRYsTPHAF |
| 656. | HTAsPTGMMK |
| 657. | HTFsPSPKL |
| 658. | HTIsPLDL |
| 659. | HTIsPSFQL |
| 660. | HVSLItPTKR |
| 661. | HVYtPSTTK |
| 662. | HYSsLVRVL |
| 663. | HYSsRLGSAIF |
| 664. | IADDRQsL |
| 665. | IAKsPHSTV |
| 666. | IAQDHRSsL |
| 667. | IEKIyIMKADTVIVG |
| 668. | IGKMRYVsV |
| 669. | HEtPHKEI |
| 670. | IIEtPHKEY |
| 671. | IIHsLETKL |
| 672. | IIQsPSSTGLLK |
| 673. | IISsPLKGY |
| 674. | IISsPLTGK |
| 675. | ILDISEHtL |
| 676. | ILDRtPEKL |
| 677. | ILDRtPEKV |
| 678. | ILDsGIYRI |
| 679. | ILDsGIYRV |
| 680. | ILGPPPPsFHL |
| 681. | ILKPRRsL |
| 682. | ILKsPEIQRA |
| 683. | ILKsPEIQRV |
| 684. | ILQtPQFQM |
| 685. | ILQVsIPSL |
| 686. | ILYPRPKsL |
| 687. | IMDRtPEKL |
| 689. | IMDsGIYRI |
| 690. | IMDsGIYRV |
| 691. | IMKsPEIQRA |
| 692. | IMKsPEIQRV |
| 693. | INKERRSsL |
| 694. | IPAPPSSPL |
| 695. | IPHQRSsL |
| 696. | IPIsLHTSL |
| 697. | IPKSKFLAL |
| 698. | IPLSKIKtL |
| 699. | IPRPLSLIGSTL |
| 700. | IPRsPFKVKVL |
| 701. | IPRTPLSPSPM |
| 702. | IPSSPQKVAL |
| 703. | IPTsPTSKY |
| 704. | IPTsSVLSL |
| 705. | IPVsKPLSL |
| 706. | IPVSKPLsL |
| 707. | IPVsPHIY |
| 708. | IPVSSHNSL |
| 709. | IPVSSHNSL |
| 710. | IPYAPsGEIPK |
| 711. | IQFsPPFPGA |
| 712. | IRAsLTKHF |
| 713. | IRFGRKPs |
| 714. | IRFGRKPsL |
| 715. | IRGsKIRFL |
| 716. | IRKERPIsL |
| 717. | IRNsQTRKI |
| 718. | IRSsYIRVL |
| 719. | IRYSGHsL |
| 720. | ISDGtLKY |
| 721. | ISDGtPLKY |
| 722. | ISDSAHtDY |
| 723. | ISDsMHSLY |
| 724. | ISDtPHKEI |
| 725. | ISDtPHKEY |
| 726. | ISEGtLKY |
| 727. | ISEGtPLKY |
| 728. | ISESAHtDY |
| 729. | ISEsMHSLY |
| 730. | ISEtPHKEI |
| 731. | ISEtPHKEY |
| 732. | ISFSAHtDY |
| 733. | ISIDsPQKL |
| 734. | ISNsHPLSL |
| 735. | ISSsMHSLY |
| 736. | IStDRDPL |
| 737. | IStDRDPY |
| 738. | IsTSPSVAL |
| 739. | IStSPSVAL; ISTsPSVAL; |
| | IstSPSVAL; IsTsPSVAL; |
| | IStsPSVAL; IstsPSVAL |
| 740. | ISVsPLATSAL |
| 741. | ISVSRSTsF |
| 742. | ITDGtLKY |
| 743. | ITDGtPLKY |
| 744. | ITDLPDHLLsY |
| 745. | ITDSAHtDY |
| 746. | ITDsMHSLY |
| 747. | ITDtPHKEI |
| 748. | ITDtPHKEY |
| 749. | ITEGtLKY |
| 750. | ITEGtPLKY |
| 751. | ITESAHtDY |
| 752. | ITEsMHSLY |
| 753. | ITEtPHKEI |
| 754. | ITEtPHKEY |
| 755. | ITItPPDRY |
| 756. | ITQGtLKY |
| 757. | ITQGtPLKK |
| 758. | ITQGtPLKY |
| 759. | ITtDRDPL |
| 760. | ITtDRDPY |
| 761. | ITTsPITVRK |
| 762. | ITYMsPAKL |
| 763. | IVDsPEKL |
| 764. | IVLsDSEVIQL |
| 765. | IVRyHQL |
| 766. | IVSsLRLAY |
| 767. | IVtDRDPL |
| 768. | IVtDRDPY |
| 769. | IYQyIQSRF |
| 770. | IYRSQsPHYF |
| 771. | IYYKsMPNL |
| 772. | IYYQsPLSL |
| 773. | KADsLEVQQM |
| 774. | KADtVSKTEL |
| 775. | KAFsPVR |
| 776. | KAFsPVRS |
| 777. | KAFsPVRSV |
| 778. | KAFsPVRSV; kAFsPVRSV |
| 779. | KAFsPVRSVR |
| 780. | KAFsPVRSVRK |
| 781. | KAKsPAPGL |
| 782. | KAKsPAPGV |
| 783. | KAPsPPPLL |
| 784. | KAPsRQISL; KAPSRQIsL; |
| | KAPsRQIsL |
| 785. | KARsPGRAF |
| 786. | KARsPGRAL |
| 787. | KARsPGRAM |
| 788. | KARsPGRAV |
| 789. | KASPKRLsL |
| 790. | KAVsLFLCY |
| 791. | KAVSLFLCY; KAVsLFLcY |
| 792. | KEDsDEVHL |
| 793. | KEGEEPTVYsDEEEPKDESA |
| | RKND |
| 794. | KEKsPFRET |
| 795. | KEKTIHLtL |
| 796. | KELARQIsF |
| 797. | KELsPAGSI |
| 798. | KEMsPTRQF |
| 799. | KEMsPTRQL |
| 800. | KEMsPTRQW |
| 801. | KEMsPTRQY |
| 802. | KEQsPEPHL |
| 803. | KESsPLSSRKI |
| 804. | KEStLHLVL |
| 805. | KEtPDKVEL |
| 806. | KEVDPsTGELQSL; |
| | KEVDPStGELQSL; |
| | KEVDPstGELQSL; |
| | KEVDPSTGELQsL |
| 807. | KFLsPAQYLY |
| 808. | KFsPVRSV |
| 809. | KGFsGTFQL |
| 810. | KGIsSSSLKEK |
| 811. | KIAsEIAQL |
| 812. | KIDsPTKV |
| 813. | KIDsPTKVK |
| 814. | KIDsPTKVKK |
| 815. | KIEKIyIMKADTVIVG |
| 816. | KIEsLENLYL |
| 817. | KIFsGVFVK |
| 818. | KIFsGVFVKV |
| 819. | KIFsKQQGK |
| 820. | KIFsKQQGY |
| 821. | KIGsIIFQV |
| 822. | KIHtLELKL |
| 823. | KIIsIFSG |
| 824. | KIIsIFSGTEK |
| 825. | KIKsFEVVF |
| 826. | KIKsFVKVY |
| 827. | KIKsLEEIYL |
| 828. | KIMsPRKAL |
| 829. | KIMSSPLSK |
| 830. | KIMSSPLSK |
| 831. | KIMssPLSK |
| 832. | KIRPHIAtL |
| 833. | KIRSSPREAK |
| 834. | KIRSSPREAY |
| 835. | KIRTsPTFR |
| 836. | KIRTsPTFY |
| 837. | KIsSLEIKL |
| 838. | KISsLEIKL |
| 839. | KIssLEIKL |
| 840. | KLAsLEREASV |
| 841. | KLAsLLHQV |
| 842. | KLAsPEKLAGL |
| 843. | KLAsPELERL |
| 844. | KLAsPELERV |
| 845. | KLAsPSEVVQQV |
| 846. | KLDIVSSQKV |
| 847. | KLDsFLDMQV |
| 848. | KLDsPRVTV |
| 849. | KLDsPTKVKK |
| 850. | KLDsPTKVKY |
| 851. | KLFHGsLEEL |
| 852. | KLFPDtPLAL |
| 853. | KLFPDtPLAV |
| 854. | KLFsGTVRK |
| 855. | KLFsGVFVKV |
| 856. | KLFsKQQGK |
| 857. | KLFsKQQGY |
| 858. | KLFsPAHKK |
| 859. | KLFsPAHKY |
| 860. | KLFsPSKEAEL |
| 861. | KLFsPSKEAEV |
| 862. | KLHGsLARAGK |
| 863. | KLHGsLARAGY |
| 864. | KLHsLIGLGI |
| 865. | KLIDIVsSQKV |
| 866. | KLIDRTEsL |
| 867. | KLIDVsSQKV |
| 868. | KLIsSSSLKEK |
| 869. | KLIsSSSLKEY |
| 870. | KLKDRLPsI |
| 871. | KLKSNPDFLK |
| 872. | KLKsNPDFLKK |
| 873. | KLKsNPDFLKY |
| 874. | KLKsPAPGL |
| 875. | KLKsPAPGV |
| 876. | KLKsQEIFL |
| 877. | KLKSsPLIEKK |
| 878. | KLKSsPLIEKY |
| 879. | KLKtPLVAK |
| 880. | KLKtPLVAR |
| 881. | KLLDFGSLSNL |
| 882. | KLLDFGsLSNLQV; |
| | KLLDFGSLsNLQV |
| 883. | KLLQFYPsL |
| 884. | KLLQFYPsV |
| 885. | KLLsPSDEKL |
| 886. | KLLsPSNEKV |
| 887. | KLLSSAQRtL |
| 888. | KLLSSAQRtV |
| 889. | KLLsTEEMEL |
| 890. | KLLsTEEMEV |
| 891. | KLLsVERIK |
| 892. | KLLsYIQRL |
| 893. | KLLtPIKEK |
| 894. | KLLtPIKEY |
| 895. | KLMAPDIsL |
| 896. | KLMAPDIsV |
| 897. | KLMIDRTEsV |
| 898. | KLMsDVEDV |
| 899. | KLMsPKADV |
| 900. | KLMsPKADVKL |
| 901. | KLMsPKADVKV |
| 902. | KLPDsPALA |
| 903. | KLPDsPALAK |
| 904. | KLPDsPALAKK |
| 905. | KLPDsPALAKY |
| 906. | KLPDsPALAY |
| 907. | KLPsGSKKV |
| 908. | KLPsPAPARK |
| 909. | KLPTsPLKMK |
| 910. | KLPTsPLKMY |
| 911. | KLPTtPVKAK |
| 912. | KLPTtPVKAY |
| 913. | KLQEFLQtL |
| 914. | KLQVtSLSV |
| 915. | KLRsPFLQK |
| 916. | KLRsPFLQY |
| 917. | KLRsPKSEL |
| 918. | KLRSSPREAK |
| 919. | KLRTsPTFK |
| 920. | KLsGDQPAAR |
| 921. | KLSGLsF |
| 922. | KLSSLGNLK |
| 923. | KLSsLGNLKK |
| 924. | KLSSLGNLKY |
| 925. | KLSsPRGGMK |
| 926. | KLSsPRGGMKK |
| 927. | KLSsPRGGMKY |
| 928. | KLsVIAED SESGKQN |
| 929. | KLsVIAEDSESGKQNP |
| 930. | KLsVIAED SESGKQNPG |
| 931. | KLVSFHDDsDEDL |
| 932. | KLwtLVSEQTRV |
| 933. | KLYsEIDIKV |
| 934. | KLYsGNMEK |
| 935. | KLYsISSQV |
| 936. | KLYTyIQSR |
| 937. | KLYTyIQSRF |
| 938. | KMAsLARKV |
| 939. | KMAsLLHQV |
| 940. | KMAsPELERL |
| 941. | KMAsPELERV |
| 942. | KMDIVSSQKV |
| 943. | KMDsFLDMQL |
| 944. | KMDsFLDMQV |
| 945. | KMDsPRVTV |
| 946. | KMDsPTKVKK |
| 947. | KMFPDtPLAL |
| 948. | KMFPDtPLAV |
| 949. | KMFsGTVRK |
| 950. | KMFsGVFVKV |
| 951. | KMFsKQQGK |
| 952. | KMFsPAHKK |
| 953. | KMFsPSKEAEL |
| 954. | KMFsPSKEAEV |
| 955. | KMHGsLARAGK |
| 956. | KMIDIVsSQKV |
| 957. | KMIDRTEsL |
| 958. | KMIsSSSLKEK |
| 959. | KMKSNPDFLK |
| 960. | KMKsNPDFLKK |
| 961. | KMKsNPDFLKY |
| 962. | KMKSsPLIEKK |
| 963. | KMKtPLVAK |
| 964. | KMKtPLVAR |
| 965. | KMLDFGSLsNLQV |
| 966. | KMLQFYPsL |
| 967. | KMLsCAGADRL |
| 968. | KMLscAGADRL |
| 969. | KMLsPSNEKL |
| 970. | KMLsPSNEKV |
| 971. | KMLSSAQRtL |
| 972. | KMLSSAQRtV |
| 973. | KMLsVERIK |
| 974. | KMLtPIKEK |
| 975. | KMLtPRIEL |
| 976. | KMMAPDIsV |
| 977. | KMMsPKADVKL |
| 978. | KMMsPKADVKV |
| 979. | KMPTsPLKMK |
| 980. | KMPTtPVKAK |
| 981. | KMPTtPVKAY |
| 982. | KMRsPFLQK |
| 983. | KMRSSPREAK |
| 984. | KMRTsPTFK |
| 985. | KMSSLGNLK |
| 986. | KMSsLGNLKK |
| 987. | KMSsLGNLKY |
| 988. | KMSSPRGGMK |
| 989. | KMSsPRGGMKK |
| 990. | KMsSYAFFV |
| 991. | KMSsYAFFV |
| 992. | KMssYAFFV |
| 993. | KMVsMKPPGF |
| 994. | KMYsEIDIKV |
| 995. | KMYsGNMEK |
| 996. | KNRsWKYN |
| 997. | KNRsWKYNQ |
| 998. | KNRsWKYNQSISLR |
| 999. | KNRsWKYNQSISLRRP |
| 1000. | KPAsPARRF |
| 1001. | KPASPARRL |
| 1002. | KPAsPARRLDL |
| 1003. | KPAsPARRM |
| 1004. | KPAsPARRV |
| 1005. | KPAsPKFIVTF |
| 1006. | KPAsPKFIVTL |
| 1007. | KPAsPKFIVTM |
| 1008. | KPAsPKFIVTV |
| 1009. | KPAVSRsRSSSL |
| 1010. | KPEsRRSSLL |
| 1011. | KPFKLSGLsF |
| 1012. | KPGLGEGtP |
| 1013. | KPLIRSQsL |
| 1014. | KPMtPKVVTL |
| 1015. | KPPGtPPPSAL |
| 1016. | KPPHsPLVF |
| 1017. | KPPHsPLVL |
| 1018. | KPPHsPLVM |
| 1019. | KPPHsPLVV |
| 1020. | KPPsPEHQSF |
| 1021. | KPPsPEHQSL |
| 1022. | KPPsPEHQSM |
| 1023. | KPPsPEHQSV |
| 1024. | KPPsPGTVL |
| 1025. | KPPsPGTVLAL |
| 1026. | KPPsPSPIEF |
| 1027. | KPPsPSPIEM |
| 1028. | KPPsPSPIEV |
| 1029. | KPPtPGASF |
| 1030. | KPPtPGASL |
| 1031. | KPPPGASM |
| 1032. | KPPtPGASV |
| 1033. | KPPtSQSSVL; KPPTsQSSVL |
| 1034. | KPPVsFFSL |
| 1035. | KPPYRSHsF |
| 1036. | KPPYRSHsL |
| 1037. | KPPYRSHsM |
| 1038. | KPPYRSHsV |
| 1039. | KPQTRGKtF |
| 1040. | KPQTRGKtL |
| 1041. | KPQTRGKtM |
| 1042. | KPQTRGKtV |
| 1043. | KPRPLsMDL |
| 1044. | KPRPPPLsF |
| 1045. | KPRPPPLsL |
| 1046. | KPRPPPLsM |
| 1047. | KPRPPPLsP |
| 1048. | KPRPPPLsV |
| 1049. | KPRRFsRSL |
| 1050. | KPRsPDHVF |
| 1051. | KPRsPDHVL |
| 1052. | KPRsPDHVM |
| 1053. | KPRsPDHVV |
| 1054. | KPRsPFSKI |
| 1055. | KPRsPPRAF |
| 1056. | KPRsPPRAL |
| 1057. | KPRsPPRALF |
| 1058. | KPRsPPRALL |
| 1059. | KPRsPPRALM |
| 1060. | KPRsPPRALV |
| 1061. | KPRsPPRALVF |
| 1062. | KPRsPPRALVL |
| 1063. | KPRsPPRALVLF |
| 1064. | KPRsPPRALVLL |
| 1065. | KPRsPPRALVLM |
| 1066. | KPRsPPRALVLP |
| 1067. | KPRsPPRALVLV |
| 1068. | KPRsPPRALVM |
| 1069. | KPRsPPRALVV |
| 1070. | KPRsPPRAM |
| 1071. | KPRsPPRAV |
| 1072. | KPRsPVVEF |
| 1073. | KPRsPVVEL |
| 1074. | KPRsPVVEM |
| 1075. | KPRsPVVEV |
| 1076. | KPSsLRRVTI |
| 1077. | KPSsPRGSL |
| 1078. | KPSsPRGSLL |
| 1079. | KPTLYnVSL |
| 1080. | KPVsPKSGTL |
| 1081. | KPVsPLLL |
| 1082. | KPYsPLASF |
| 1083. | KPYsPLASL |
| 1084. | KPYsPLASM |
| 1085. | KPYsPLASV |
| 1086. | KQDsLVINL |
| 1087. | KQKsLTNLSF |
| 1088. | KQPsFSAKKM |
| 1089. | KQYsGKFF |
| 1090. | KRAsALLNL |
| 1091. | KRAsFAKSF |
| 1092. | KRAsFAKSK |
| 1093. | KRAsFAKSL |
| 1094. | KRASFAKSM |
| 1095. | KRAsFAKSR |
| 1096. | KRAsFAKSV |
| 1097. | KRAsFAKSY |
| 1098. | KRAsGQAFEF |
| 1099. | KRAsGQAFEK |
| 1100. | KRAsGQAFEL |
| 1101. | KRAsGQAFER |
| 1102. | KRAsGQAFEY |
| 1103. | KRAsRIYNT |
| 1104. | KRASsPFRF |
| 1105. | KRASsPFRK |
| 1106. | KRASsPFRL |
| 1107. | KRASSPFRM |
| 1108. | KRASsPFRR |
| 1109. | KRASsPFRY |
| 11I0. | KRAsVFVKF |
| 1111. | KRAVFVKK |
| 1112. | KRAsVFVKL |
| 1113. | KRASVFVKM |
| 1114. | KRAsVFVKR |
| 1115. | KRAsVFVKY |
| 1116. | KRAsYILRL |
| 1117. | KRFsFKF |
| 1118. | KRFsFKK |
| 1119. | KRFsFKKSF |
| 1120. | KRFsFKKsFKL; |
| | KRFsFKKsFKL |
| 1121. | KRFsFKKSK |
| 1122. | KRFsFKKSL |
| 1123. | KRFSFKKSM |
| 1124. | KRFsFKKSR |
| 1125. | KRFsFKKSY |
| 1126. | KRFsFKL |
| 1127. | KRFSFKM |
| 1128. | KRFsFKR |
| 1129. | KRFsFKY |
| 1130. | KRFsGTVRF |
| 1131. | KRFsGTVRK |
| 1132. | KRFsGTVRL |
| 1133. | KRFSGTVRM |
| 1134. | KRFsGTVRR |
| 1135. | KRFsGTVRY |
| 1136. | KRFsLDFNL |
| 1137. | KRIsIFLSM |
| 1138. | KRIsISTSGGSF |
| 1139. | KRIsRMRLV |
| 1140. | KRKsFTSLY |
| 1141. | KRLEKSPsF |
| 1142. | KRLEKsPSF |
| 1143. | KRLSPAPQF |
| 1144. | KRLSPAPQK |
| 1145. | KRLSPAPQL |
| 1146. | KRLSPAPQM |
| 1147. | KRLSPAPQR |
| 1148. | KRLSPAPQY |
| 1149. | KRLsTSPVRL |
| 1150. | KRLsVELTSSL |
| 1151. | KRLsVELTSSLF |
| 1152. | KRLsVERIF |
| 1153. | KRLsVERIK |
| 1154. | KRLSVERIL |
| 1155. | KRLSVERIM |
| 1156. | KRLsVERIR |
| 1157. | KRLsVERIY |
| 1158. | KRLsVERIYQK |
| 1159. | KRLtHVYDL |
| 1160. | KRMsNELENY |
| 1161. | EKRMsPKEF |
| 1162. | KRMsPKEK |
| 1163. | KRMsPKEL |
| 1164. | KRMsPKER |
| 1165. | KRMsPKEY |
| 1166. | KRMsPKPEL |
| 1167. | KRMsPKPF |
| 1168. | KRMsPKPK |
| 1169. | KRMsPKPL |
| 1170. | KRMSPKPM |
| 1171. | KRMSPKPR |
| 1172. | KRMSPKPR |
| 1173. | KRMsPKPY |
| 1174. | KRMsVTEGGIKY |
| 1175. | KRNsIKKIV |
| 1176. | KRNsRLGFL |
| 1177. | KRNtFVGTPF |
| 1178. | KRRtGALVL |
| 1179. | KRsPIFF |
| 1180. | KRsSISQLL; KRSsISQLL; |
| | KRssISQLL |
| 1181. | KRSsVHGVSF |
| 1182. | KRTsKYFSL |
| 1183. | KRWQsPVTK |
| 1184. | KRYsEPVSL |
| 1185. | KRYsGNMEF |
| 1186. | KRYsGNMEK |
| 1187. | KRYSGNMEL |
| 1188. | KRYsGNMEM |
| 1189. | KRYSGNMER |
| 1190. | KRYsGNMEY |
| 1191. | KRYsRALYL |
| 1192. | KRYsRSLTI |
| 1193. | KSDGsFIGY |
| 1194. | KSDsPAIQL |
| 1195. | KSDsPSTSSI |
| 1196. | KSDsRQERY |
| 1197. | KSGELLAtW |
| 1198. | KSKsMDLGI |
| 1199. | KSKsNPDFLKK |
| 1200. | KSKtPLVAK |
| 1201. | KSKtPLVAR |
| 1202. | KSKtPLVAY |
| 1203. | KSLsPSGLKI |
| 1204. | KSLsPSLLGY |
| 1205. | KsLVRLLLL |
| 1206. | KSPTsPLNM |
| 1207. | KSsIIIRM |
| 1208. | KsSSLDKQL; KSsSLDKQL; |
| | KSSsLDKQL; KsssLDKQL |
| 1209. | KSSsLGNLKK |
| 1210. | KsVKALSSLHGDDQ |
| 1211. | KsVKALSSLHGDDQD |
| 1212. | KSVKALSSLHGDDQDsEDE |
| 1213. | KSYsFIARMKA |
| 1214. | KSYsRSRsR |
| 1215. | KTDGsFIGY |
| 1216. | KTDsRQERY |
| 1217. | KTEsPRTSGVL |
| 1218. | KTEsRQERY |
| 1219. | KTFsIGKIAK |
| 1220. | KTIsLTDFL |
| 1221. | KTKsIAEEL |
| 1222. | KTKsMFFFL |
| 1223. | KTLsLVKEL |
| 1224. | KtLSPGKNGVVK |
| 1225. | KTMsGTFLL |
| 1226. | KTMsGTFLL |
| 1227. | KTMsPSQMIM |
| 1228. | KTPsHTRML |
| 1229. | KTPsLTRRI |
| 1230. | KTPTsPLKM |
| 1231. | KTPTsPLKMK |
| 1232. | KTPTsPLKMY |
| 1233. | KTQsLPVTEK |
| 1234. | KTRsLSVEI |
| 1235. | KTRsLSVEIVY; |
| | KTRSLsVEIVY; |
| | KTRsLsVEIVY |
| | KVYsssEFL |
| 1236. | KTVsEPNLKL |
| 1237. | KTRsLSVEIVY |
| 1238. | KTVsPSPAF |
| 1239. | KTWKGsIGL |
| 1240. | KVAsLLHQV |
| 1241. | KVDsPTVTTTL |
| 1242. | KVDsPVIF |
| 1243. | KVHGsLARAGK |
| 1244. | KVHGsLARAGY |
| 1245. | KVIPVTRsL |
| 1246. | KVKSsPLIEKK |
| 1247. | KVKSsPLIEKL |
| 1248. | KVKSsPLIEKY |
| 1249. | KVLsSLVTL; KVLSsLVTL; |
| | KVLssLVTL |
| 1250. | KVLsKEFHL |
| 1251. | KVLSPtAAK |
| 1252. | KVLsSLVTL |
| 1253. | KVLsTEEMEL; |
| | KVLStEEMEL |
| 1254. | KVLtPIKEK |
| 1255. | KVLsTEEMEL |
| 1256. | KVLtPIKEY |
| 1257. | KVPDsPALAK |
| 1258. | KVPDsPALAKK |
| 1259. | KVPDsPALAKY |
| 1260. | KVPDsPALAY |
| 1261. | KVPTsPLKMY |
| 1262. | KVQsLRRAL |
| 1263. | KVQVtSLSV |
| 1264. | KVYsSSEFL; KVYSsSEFL; |
| | KTRSLsVEIVY; |
| | KTRsLsVEIVY |
| | KVYsssEFL |
| 1236. | KTVsEPNLKL |
| 1237. | KTRsLSVEIVY |
| 1238. | KTVsPSPAF |
| 1239. | KTWKGsIGL |
| 1240. | KVAsLLHQV |
| 1241. | KVDsPTVTTTL |
| 1242. | KVDsPVIF |
| 1243. | KVHGsLARAGK |
| 1244. | KVHGsLARAGY |
| 1245. | KVIPVTRsL |
| 1246. | KVKSsPLIEKK |
| 1247. | KVKSsPLIEKL |
| 1248. | KVKSsPLIEKY |
| 1249. | KVLsSLVTL; KVLSsLVTL; |
| | KVLssLVTL |
| 1250. | KVLsKEFHL |
| 1251. | KVLSPtAAK |
| 1252. | KVLsSLVTL |
| 1253. | KVLsTEEMEL; |
| | KVLStEEMEL |
| 1254. | KVLtPIKEK |
| 1255. | KVLsTEEMEL |
| 1256. | KVLtPIKEY |
| 1257. | KVPDsPALAK |
| 1258. | KVPDsPALAKK |
| 1259. | KVPDsPALAKY |
| 1260. | KVPDsPALAY |
| 1261. | KVPTsPLKMY |
| 1262. | KVQsLRRAL |
| 1263. | KVQVtSLSV |
| 1264. | KVYsSSEFL; KVYSsSEFL; |
| | KVYSSsEFL; KVYssSEFL; |
| | KVYSssEFL; KVYsSsEFL; |
| 1265. | KVYtPSISK |
| 1266. | KYELsVIM |
| 1267. | KYIsGPHEL |
| 1268. | KYPDVAsPTL |
| 1269. | KYsPGKLRGN |
| 1270. | LADsPLKL |
| 1271. | LALTRSSSL |
| 1272. | LDEAGQRStM |
| 1273. | LEAPPsPSL |
| 1274. | LEItPPSSEKL |
| 1275. | LESPTtPLL; LESPttPLL; |
| | LESPTtPLL; LEsPTTPLL |
| 1276. | LGGGGAGLSGRASGGAQsP |
| | LRYLHV |
| 1277. | LIDNsFNRY |
| 1278. | LIMPRPNsV |
| 1279. | LKLsYLTWV |
| 1280. | LLARtPPAA |
| 1281. | LLASPGHISV |
| 1282. | LLDPSRSYsY |
| 1283. | LLDtPVKTQY |
| 1284. | LLFsPVTSL |
| 1285. | LLFsPVTSV |
| 1286. | LLLsEEVEL |
| 1287. | LLNKSSPVK |
| 1288. | LLNKSSPVKK |
| 1289. | LLNKSsPVKY |
| 1290. | LLNKtPPTA |
| 1291. | LMFsPVTSL |
| 1292. | LMFsPVTSV |
| 1293. | LMHsFILKA |
| 1294. | LMNKSSPVK |
| | LPLSSsHLNVY; |
| | LPLSSSHLNVY; |
| | LPLsSsHLNVY; |
| | LPLSssHLNVY; |
| | LPLsssHLNVY |
| 1295. | LMNKSsPVKK |
| 1296. | LMNKSsPVKY |
| 1297. | LPAFKRKtL |
| 1298. | LPAsPAGRL |
| 1299. | LPAsPAHQL |
| 1300. | LPAsPHQF |
| 1301. | LPASPHQL |
| 1302. | LPAsPHQM |
| 1303. | LPAsPHQV |
| 1304. | LPAsPRARF |
| 1305. | LPAsPRARL |
| 1306. | LPAsPRARLSA |
| 1307. | LPAsPRARM |
| 1308. | LPAsPRARV |
| 1309. | LPAsPSVSL |
| 1310. | LPAsPVARR |
| 1311. | LPDPGsPRL |
| 1312. | LPEsPRLTL |
| 1313. | LPIFSRLsF |
| 1314. | LPIFSRLsI |
| 1315. | LPIFSRLsL |
| 1316. | LPIFSRLsM |
| 1317. | LPIFSRLsV |
| 1318. | LPKARPMsL |
| 1319. | LPKGLSAsL |
| 1320. | LPKGLsASL |
| 1321. | LPKSPPYTAF |
| 1322. | LPKsPPYTAL |
| 1323. | LPKsPPYTAM |
| 1324. | LPKsPPYTAV |
| 1325. | LPLsPKETV |
| 1326. | LPLsSSHLNVY; |
| | LPLSSSHLNVY; |
| 1327. | LPNsIASRF |
| 1328. | LPRGSsPSVF |
| 1329. | LPRGSsPSVL |
| 1330. | LPRGSsPSVM |
| 1331. | LPRGSsPSW |
| 1332. | LPRMIsHSEL |
| 1333. | LPRNsTMM; LPRNStMM |
| 1334. | LPRPAsPAL |
| 1335. | LPRPLsPTKL |
| 1336. | LPRPLSPtKL; LPRPLsPtKL |
| 1337. | LPRsPRLGH |
| 1338. | LPRSSsMAA |
| 1339. | LPRSSsMAAGL |
| 1340. | LPRtPRPEL |
| 1341. | LPRtPSASSL; LPRTPsASSL; |
| | LPRtPsASSL |
| 1342. | LPRtPSYSI |
| 1343. | LPSESVSsL |
| 1344. | LPsPRGQRVI |
| 1345. | LPsPTATSQL |
| 1346. | LPSSGRSsL |
| 1347. | LPTsLPSSL |
| 1348. | LPTsPLAMEY |
| 1349. | LPVsPGHRKT |
| 1350. | LPVsPRLQL |
| 1351. | LPYPVsPKQKY |
| 1352. | LQHSFsFAGF |
| 1353. | LQIsPPLHQHL |
| 1354. | LQIsPVSSY |
| 1355. | LQIsPVSSYA |
| 1356. | LQLPsPTAT |
| 1357. | LQLsPLKGLSL |
| 1358. | LQNItENQL |
| 1359. | LSAsFRSLY |
| 1360. | LSAsPLTSL |
| 1361. | LSDDGKAsL |
| 1362. | LSDPSRSYsY |
| 1363. | LSDsDTEAKL |
| 1364. | LSDsDTEAKY |
| 1365. | LSDsPSMGRY |
| 1366. | LSDtPVKTQY |
| 1367. | LSEIKFNsY |
| 1368. | LSEPSRSYsY |
| 1369. | LSEsDTEAKL |
| 1370. | LSEsDTEAKY |
| 1371. | LSEtPVKTQY |
| 1372. | LSKFRMPQPSSGREsPRH |
| 1373. | LSKsEHSLF |
| 1374. | LSSsPPATHF |
| 1375. | LSSsVIREL |
| 1376. | LTDPSRSYsY |
| 1377. | LTDPSsPTIS |
| 1378. | LTDPSsPTISSY |
| 1379. | LTDsDTEAKL |
| 1380. | LTDsDTEAKY |
| 1381. | LTDtPVKTQY |
| 1382. | LTEPSRSYsY |
| 1383. | LTEsDTEAKL |
| 1384. | LTEsDTEAKY |
| 1385. | LTEtPVKTQY |
| 1386. | LTHsLVLHY |
| 1387. | LTKsPLAQM |
| 1388. | LTLsPKLQL |
| 1389. | LTSsRLLKL |
| 1390. | LTYRRRLsY |
| 1391. | LVAsPRLEK |
| 1392. | LVDsVAKTM |
| 1393. | LVVsPGQQTL |
| 1394. | LYTyIQSRF |
| 1395. | MLAEsPSVPRL |
| 1396. | MLAEsPSVPRV |
| 1397. | MLPsILNQL |
| 1398. | MLRsPPRVSK |
| 1399. | MMRsPPRVSK |
| 1400. | MPGSPTKTVY |
| 1400. | MPHsPTLRV |
| 1402. | MPKFRMPsL |
| 1403. | MPLsPDPSHTTL |
| 1404. | MPMRsPSKL |
| 1405. | MPNsPAPHF |
| 1406. | MPREPsATRL |
| 1407. | MPRPsIKKAQNSQAARQ |
| 1408. | MPRQPsATRF |
| 1409. | MPRQPsATRL |
| 1410. | MPRQPsATRM |
| 1411. | MPRQPsATRV |
| 1412. | MPsPATLSHSL |
| 1413. | MPsPGGRITL |
| 1414. | MPSPVsPKL |
| 1415. | MPVPtTPEF |
| 1416. | MPVPTtPEF |
| 1417. | MPVPttPEF |
| 1418. | MPVRPTtNTF |
| 1419. | MPVtSSSFF |
| 1420. | MRLsRELQF |
| 1421. | MRLSRELQK |
| 1422. | MRLsRELQL |
| 1423. | MRLSRELQM |
| 1424. | MRLsRELQR |
| 1425. | MRLsRELQY |
| 1426. | MSDtYRLKY |
| 1427. | MSEtYRLKY |
| 1428. | MTDtYRLKY |
| 1429. | MTEtYRLKY |
| 1430. | MTKSsPLKI |
| 1431. | MTKsSPLKI |
| 1432. | MTKssPLKI |
| 1433. | MTRsPPRVSK |
| 1434. | MTRsPPRVSY |
| 1435. | NAEsGRGQVM |
| 1436. | NAIsLPTI |
| 1437. | NEFHsPIGL |
| 1438. | NFKsPVKTIR |
| 1439. | NGIIRSQsF |
| 1440. | NIAsPGTVHKR |
| 1441. | NIPsFIVRL |
| 1442. | NLELSKFRMPQP |
| | SSGREsPRH |
| 1443. | NLGsRNHVHQL |
| 1444. | NLIsPVRNGAV |
| 1445. | NLLsPDGKMISV |
| 1446. | NLVERKNsK |
| 1447. | NLVERKNSK |
| 1448. | NLVERKNsL |
| 1449. | NMDsPGPML |
| 1450. | NMVERKNsK |
| 1451. | NMVERKNsL |
| 1452. | NPsSPEFFM; NPSsPEFFM; |
| | NPssPEFFM |
| 1453. | NPVsLPSL |
| 1454. | NRAMRRVSSVPSR |
| 1455. | NRAMRRVsSVPSRAQ |
| 1456. | NRFsPKASL |
| 1457. | NRLsKGLQI |
| 1458. | NRMsRRIVL |
| 1459. | NRRKsALAL |
| 1460. | NRRsPPPSL |
| 1461. | NRsWKYNQSISLR |
| 1462. | NRsWKYNQSISLRRP |
| 1463. | NRYtNRVVTF |
| 1464. | NRYNRVVTK |
| 1465. | NRYtNRVVTL |
| 1466. | NRYTNRVVTM |
| 1467. | NRYtNRVVTR |
| 1468. | NRYtNRVVTY |
| 1469. | NSDLPtSPL; NSDLPTsPL; |
| | NSDLPtsPL |
| 1470. | NSDsPLRY |
| 1471. | NSEsPLRY |
| 1472. | NSLsPRSSL |
| 1473. | NSVsPSESL |
| 1474. | NTDsPLRY |
| 1475. | NTEsPLRY |
| 1476. | NYQLsPTKL |
| 1477. | NYVERKNsK |
| 1478. | NYVERKNsL |
| 1479. | NYVERKNsY |
| 1480. | PEVsPRPAL |
| 1481. | PFKVsPLTF |
| 1482. | PIFNRIsV |
| 1483. | PIFPMARsI |
| 1484. | PLVSSSDsPPRPQPAF |
| 1485. | PMVTLsLNL |
| 1486. | PPLPEDSIKVIRNMRAAsP |
| 1487. | PPStSAAAL; PPSTsAAAL; |
| | PPsTSAAAL |
| 1488. | PRFsLDAEIDSL |
| 1489. | PRPANsGGVDL |
| 1490. | PRPsPGSNSKV |
| 1491. | PRPsPRQNSI |
| 1492. | PRQRAtSNVF |
| 1493. | PRsPPRAL |
| 1494. | PRWsPAVSA |
| 1495. | PSPPsPLEKTPL |
| 1496. | PtSPLAMEY |
| 1497. | PTsPLAMEY |
| 1498. | PtsPLAMEY |
| 1499. | PVRdPTRSP |
| 1500. | PWIPPSsPTTF |
| 1501. | PYDPALGsPSR |
| 1502. | PYDPALGsPSRLF |
| 1503. | QAASNFKsPVKTIR |
| 1504. | QAFLRSVsM |
| 1505. | QEKsPKQAL |
| 1506. | QKKIsTNL |
| 1507. | QLDRIsVYY |
| 1508. | QLDsPQRALY |
| 1509. | QLEsPQRALY |
| 1510. | QLFsPKKGQK |
| 151I. | QLSLRTVsL |
| 1512. | QMFsPKKGQK |
| 1513. | QMFSPKKGQK |
| 1514. | QPQRRsLRL |
| 1515. | QPRNSLPAsPAHQL |
| 1516. | QPRsPGPDYSF |
| 1517. | QPRsPGPDYSL |
| 1518. | QPRsPGPDYSM |
| 1519. | QPRsPGPDYSV |
| 1520. | QPRsPVPSAF |
| 1521. | QPRTPHsPPL |
| 1522. | QPRtPsPLVF |
| 1523. | QPRtPSPLVF |
| 1524. | QPRTPsPLVF |
| 1525. | QPRtPSPLVL |
| 1526. | QPRtPsPLVL |
| 1527. | QPRTPsPLVL |
| 1528. | QPRtPsPLVM |
| 1529. | QPRtPSPLVM |
| 1530. | QPRTPsPLVM |
| 1531. | QPRtPsPLVV |
| 1532. | QPRtPSPLVV |
| 1533. | QPRTPsPLVV |
| 1534. | QPSsPRVNGL |
| 1535. | QPStPDPFL |
| 1536. | QRLsPLSAAY |
| 1537. | QSDsPQRALY |
| 1538. | QSEsPQRALY |
| 1539. | QSLLsPLVL |
| 1540. | QTDsPQRALY |
| 1541. | QTEsPQRALY |
| 1542. | QTIsPLSTY |
| 1543. | QTPsPRLAL |
| 1544. | QTSIQsPSSY |
| 1545. | QVAMPVKKSPRRSsSDEQG |
| | LSYSSLKNV |
| 1546. | QVDPKKRIsM |
| | RASSDIVsL; RAssDIVSL; |
| | RAsSDIVsL; RASsDIVsL; |
| | RAssDIVsL; RASSDIVSL |
| 1547. | QVFsPKKGQK |
| 1548. | QVFsPKKGQY |
| 1549. | RAAsTARHL |
| 1550. | RAAtPLPSL |
| 1551. | RADsPGRLV |
| 1552. | RADsPVHM |
| 1553. | RADsPVHME |
| 1554. | RADsPVHMEQ |
| 1555. | RADsPVHMEQQ |
| 1556. | RAEsDFVKF |
| 1557. | RAEsPGPGSRL |
| 1558. | RAEsPTPGM |
| 1559. | RAFsFSKTPK |
| 1560. | RAFsFSKTPY |
| 1561. | RAFsVKFEV |
| 1562. | RAGsFSRFY |
| 1563. | RAHsEPLAL |
| 1564. | RAHsLARQM |
| 1565. | RAHSsPASL |
| 1566. | RAHtPTPGIYM |
| 1567. | RAIsPREKI |
| 1568. | RAKRIsQLF |
| 1569. | RAKsPISLK |
| 1570. | RAKsPISLY |
| 1571. | RALsPRVAA |
| 1572. | RALsSSVIREL |
| 1573. | RALtPSPVM |
| 1574. | RAPsPSSRF |
| 1575. | RAPsPSSRL |
| 1576. | RAPsPSSRM |
| 1577. | RAPsPSSRV |
| 1578. | RARGIsPIVF |
| 1579. | RAsSDIVSL; RASsDIVSL; |
| 1580. | RASsLSITV |
| 1581. | RASsPFRRV |
| 1582. | RAsVFVKL |
| 1583. | RAtSLPSL |
| 1584. | RATsLPSL |
| 1585. | RAtsLPSL |
| 1586. | RATsNVFAM |
| 1587. | RATsPLVSL |
| 1588. | RATsRcLQL |
| 1589. | RAVsPFAKI |
| 1590. | REtSPNRIGL; RETsPNRIGL; |
| | REtsPNRIGL |
| 1591. | REAPsPLMI |
| 1592. | REAsIELPSM |
| 1593. | REAsPAPLA |
| 1594. | REASPLSSNKLIL |
| 1595. | REAsPRLRV |
| 1596. | REAsPSRLSV |
| 1597. | REDsTPGKVFL |
| 1598. | REEsPLRIKM |
| 1599. | REGsFRVTTA |
| 1600. | REGsGRFSLP |
| | 160LREIMGtPEYL |
| 1602. | REIsSSPTS |
| 1603. | REKsPGRML |
| 1604. | REKsPLFQF |
| 1605. | REKsPLFQW |
| 1606. | REKsPLFQY |
| 1607. | RELARKGsL |
| 1608. | RELsGTIKEIL |
| 1609. | RELsPLISL |
| 1610. | RENsFGSPL |
| 1611. | RENsFGSPLEF |
| 1612. | REPsPALGPNL |
| 1613. | REPsPLPEL |
| 1614. | REPsPLPELAL |
| 1615. | REPsPVRYDNL |
| 1616. | RERsPGRLF |
| 1617. | RERsPSPSF |
| 1618. | RERWsFIRA |
| 1619. | REsPIPIEI |
| 1620. | REsPRPLQL; RESsLGFQL |
| 1621. | RESsPTRRL |
| 1622. | RETsPNRIGL |
| 1623. | REVEsLPAV; REVsPAPAV |
| 1624. | REVsPEPIV |
| 1625. | REWsPTPSL |
| 1626. | REWsPTPSSL |
| 1627. | REYGsPLKA |
| 1628. | REYGsTSSI |
| 1629. | RFKtQPVTF |
| 1630. | RFsFKKSF |
| 1631. | RGDGYGtF |
| 1632. | RGDsPKIDL |
| 1633. | RGDsRPRLV |
| 1634. | RGIsPIVF |
| 1635. | RGsFEVTL |
| 1636. | RHPKRSVsL |
| 1637. | RIDIsPSTL |
| 1638. | RIDsKDSASEL |
| 1639. | RIGsPLSPK |
| 1640. | RIHGsPLQK |
| 1641. | RILsATTSGIFL |
| 1642. | RILsGVVTK |
| 1643. | RILsGVVTKM |
| 1644. | RILsGVVTKMKM |
| 1645. | RILsGVVTY |
| 1646. | RILsKEYNM |
| 1647. | RILsPSMASK |
| 1648. | RILsPSMASY |
| 1649. | RINsFEEHV |
| 1650. | RIPsVQINF |
| 1651. | RIQsKLYRA |
| 1652. | RIQyIQSRF |
| 1653. | RIQyIQSRFY |
| 1654. | RIRPsTPSQL; RIRPStPSQL; |
| | RIRPstPSQL |
| 1655. | RIsHELDS |
| 1656. | RIStPLTGV |
| 1657. | RITsLIVHV |
| 1658. | RIVsPKNSDLK |
| 1659. | RIYQyIQ |
| 1660. | RIYQyIQSK |
| 1661. | RIYQyIQSR |
| 1662. | RIYQyIQSRF |
| 1663. | RIYQyIQSRFK |
| 1664. | RIYQyIQSRFY |
| 1665. | RIYQyIQSRK |
| 1666. | RIYQyIQSRY |
| 1667. | RIYQyIQSY |
| 1668. | RIYsMSLRL |
| 1669. | RKAsLRQFL |
| 1670. | RKLRsLEQL |
| 1671. | RKLsVILIK |
| 1672. | RKLsVILIL |
| 1673. | RKNsFVMEY |
| 1674. | RKPsAEMNRI |
| 1675. | RKPsIVTKY |
| 1676. | RKPsLAKAL |
| 1677. | RKSsIIIRM |
| 1678. | RLsSVSVTY; RLSsVSVTY; |
| | RLssVSVTY |
| 1679. | RLAsASRAL |
| 1680. | RLAsFAVRK |
| 1681. | RLASFAVRY |
| 1682. | RLAsIELPSM |
| 1683. | RLAsIELPSMAV |
| 1684. | RLAsIELPSV |
| 1685. | RLAsLMNLGM |
| 1686. | RLAsLNAEAL |
| 1687. | RLAsLNAEAV |
| 1688. | RLAsLQSEV |
| 1689. | RLAsLSISV |
| 1690. | RLAsPLVHK |
| 1691. | RLAsPLVHY |
| 1692. | RLAsPPPPPK |
| 1693. | RLAsPPPPPY |
| 1694. | RLAsPTSGV |
| 1695. | RLAsPTSGVK |
| 1696. | RLAsPTSGVKK |
| 1697. | RLASPTSGVKR |
| 1698. | RLAsPTSGVKY |
| 1699. | RLAsRPLLL |
| 1700. | RLAsSATQVHK |
| 1701. | RLASSVLRC |
| 1702. | RLAsSVLRcG |
| 1703. | RLAsYLDKV |
| 1704. | RLAsYLDRV |
| 1705. | RLAsYLSGC |
| 1706. | RLAsYLSGc |
| 1707. | RLDsIVGPQL |
| 1708. | RLDSPLSNRY |
| 1709. | RLDsTPGKVFL |
| 1710. | RLDsTPGKVFV |
| 1711. | RLDsYLRAP |
| 1712. | RLDsYVR |
| 1713. | RLDsYVR |
| 1714. | RLDsYVRS |
| 1715. | RLDsYVRSL |
| 1716. | RLDsYVRsL |
| 1717. | RLDsYVRSV |
| 1718. | RLDtGPQSL |
| 1719. | RLEsANRRL |
| 1720. | RLEsLSYQL |
| 1721. | RLFsFSKTPK |
| 1722. | RLFsHPREPAL |
| 1723. | RLFsKEL |
| 1724. | RLFsKELR |
| 1725. | RLFsKELRC |
| 1726. | RLFsKELRc |
| 1727. | RLFsKELRV |
| 1728. | RLFSLsNPSL |
| 1729. | RLFsPTYGL |
| 1730. | RLFsQGQDV |
| 1731. | RLFVGSIPK |
| 1732. | RLGsFHELL |
| 1733. | RLGsFHELLL |
| 1734. | RLIsFKAEV |
| 1735. | RLIsPYKKK |
| 1736. | RLIsQDVKL |
| 1737. | RLIsQIVSS |
| 1738. | RLIsQIVSSI |
| 1739. | RLIsQIVSSITA |
| 1740. | RLKLPSGSK |
| 1741. | RLKLPsGSKK |
| 1742. | RLKLPsGSKY |
| 1743. | RLKsDERPVHI |
| 1744. | RLKsIEERQLLK |
| 1745. | RLKsIIQEV |
| 1746. | RLKsPFRKK |
| 1747. | RLKsPGsGHVK |
| 1748. | RLKsPISLK |
| 1749. | RLKsPISLY |
| 1750. | RLKsPSPKSEK |
| 1751. | RLKsPSPKSER |
| 1752. | RLKtPTSQSYK |
| 1753. | RLKtPTSQSYR |
| 1754. | RLKTtPLRK |
| 1755. | RLKTtPLRR |
| 1756. | RLLDPSsPLAL; |
| | RLLDPsSPLAL; |
| | RLLDPssPLAL |
| 1757. | RLLDRSPsRSAK |
| 1758. | RLLDRSPsRSAY |
| 1759. | RLLsAAEN |
| 1760. | RLLsAAENFL |
| 1761. | RLLsDGQQHL |
| 1762. | RLLsDLEEL |
| 1763. | RLLsDQTRL |
| 1764. | RLLsFQRYL |
| 1765. | RLLsGVVTK |
| 1766. | RLLsGVVTY |
| 1767. | RLLsHISEA |
| 1768. | RLLsHISEV |
| 1769. | RLLsPLSSA |
| 1770. | RLLsPLSsA |
| 1771. | RLLsPLSSARL |
| 1772. | RLLsPLSSV |
| 1773. | RLLsPPLRPR |
| 1774. | RLLsPQQPAL |
| 1775. | RLLsPRPSL |
| 1776. | RLLsPRPSLL |
| 1777. | RLLsPSMASK |
| 1778. | RLLsSGVSEI |
| 1779. | RLLsSGVSEV |
| 1780. | RLLsTDAEAV |
| 1781. | RLLsVEGSTL |
| 1782. | RLLsVEIVK |
| 1783. | RLLsVEIVY |
| 1784. | RLLsVHDFDF |
| 1785. | RLLsVILIK |
| 1786. | RLLsVNIRV |
| 1787. | RLLsWSDNW |
| 1788. | RLMsGKVKV |
| 1789. | RLMsMPVAK |
| 1790. | RLMsMPVAY |
| 1791. | RLMtPKPVSI |
| 1792. | RLMtPTLSFL |
| 1793. | RLNtSDFQKL |
| 1794. | RLPNRIPsL |
| 1795. | RLPsFLKKNK |
| 1796. | RLPsLVHGY |
| 1797. | RLPSPtSPF |
| 1798. | RLPsSTLKK |
| 1799. | RLPsSTLKR |
| 1800. | RLPsSTLKY |
| 1801. | RLPRLPEI |
| 1802. | RLQsLIKNI |
| 1803. | RLQsTSERL |
| 1804. | RLQsTSERV |
| 1805. | RLQtQVFKL |
| 1806. | RLRQsPLATK |
| 1807. | RLRQsPLATR |
| 1808. | RLRQsPLATY |
| 1809. | RLRRsPLLK |
| 1810. | RLRsAGAAQK |
| 1811. | RLRsLSSLREK |
| 1812. | RLRsLssPTVTL |
| 1813. | RLRsLssPTVTV |
| 1814. | RLRsPPPVSK |
| 1815. | RLRSsLVFK |
| 1816. | RLRsSVPGV |
| 1817. | RLRSsVPGV |
| 1818. | RLRssVPGV |
| 1819. | RLRsYEDMI |
| 1820. | RLRTsPITRK |
| 1821. | RLRTSPITRR |
| 1822. | RLSDtPPLL |
| 1823. | RLsFLVSY |
| 1824. | RLsPVPVPR |
| 1825. | RLSsLIRHK |
| 1826. | RLSsLRASTSK |
| 1827. | RLSsPISKK |
| 1828. | RLSsPISKR |
| 1829. | RLSsPISKY |
| 1830. | RLSsPLHFV |
| 1831. | RLSSPVLHK |
| 1832. | RLSSPVLHR |
| 1833. | RLSsPVLHY |
| 1834. | RLSsRFSSK |
| 1835. | RLSsRFSSR |
| 1836. | RLSsRFSSY |
| 1837. | RLSsRYSQK |
| 1838. | RLSsRYSQY |
| 1839. | RLSsVKLISK |
| 1840. | RLSsVKLISY |
| 1841. | RLTFSPTYGV |
| 1842. | RLVSLSMRK |
| 1843. | RLVSLSMRY |
| 1844. | RLYKsEPEL |
| 1845. | RLYKsPLRH |
| 1846. | RLYKsPLRK |
| 1847. | RLYQyIQSK |
| 1848. | RLYQyIQSR |
| 1849. | RLYQyIQSRFK |
| 1850. | RLYQyIQSY |
| 1851. | RLYQyLQSRF |
| 1852. | RLYQyLQSRFK |
| 1853. | RLYQyLQSRFY |
| 1854. | RLYQyLQSRK |
| 1855. | RLYSGPMNKV |
| 1856. | RLYsGSRsK |
| 1857. | RLYSGSRSR |
| 1858. | RLYsGSRsY |
| 1859. | RLYsKSRDK |
| 1860. | RLYsPDHRQK |
| 1861. | RLYsPERSK |
| 1862. | RLYsPYNHK |
| 1863. | RLYsPYNHR |
| 1864. | RLYsPYNHY |
| 1865. | RLYSRsFSK |
| 1866. | RLYSRsFSY |
| 1867. | RLYsYPRQK |
| 1868. | RLYVTTSTRTYSLG |
| 1869. | RLYVTTSTRTYsLK |
| 1870. | RLYVTTSTRTYsLY |
| 1871. | RMAsPPPPPK |
| 1872. | RMAsPTSGV |
| 1873. | RMAsPTSGVK |
| 1874. | RMAsPTSGVKK |
| 1875. | RMASPTSGVKR |
| 1876. | RMAsPTSGVKY |
| 1877. | RMAsSATQVHK |
| 1878. | RMDsTPGKVFL |
| 1879. | RMDSTPGKVFV |
| 1880. | RMDsYVRSL |
| 1881. | RMDsYVRSV |
| 1882. | RMFPtPPSL |
| 1883. | RMFsFSKTPK |
| 1884. | RMFsKELRC |
| 1885. | RMFsKELRV |
| 1886. | RMFsPMEEK |
| 1887. | RMFsPMEEKELL |
| 1888. | RMFsPTYGL |
| 1889. | RMFsPTYGV |
| 1890. | RMIsKLEAQV |
| 1891. | RMISPYKKK |
| 1892. | RMIsQDVKL |
| 1893. | RMIsQDVKV |
| 1894. | RMIsTGSEL |
| 1895. | RMKLPSGSK |
| 1896. | RMKLPsGSKK |
| 1897. | RMKLPsGSKY |
| 1898. | RMKsPFRKK |
| 1899. | RMKsPGsGHVK |
| 1900. | RMKsPSPKSEK |
| 1901. | RMKSPSPKSER |
| 1902. | RMKtPTSQSYK |
| 1903. | RMKTPTSQSYR |
| 1904. | RMKTtPLRK |
| 1905. | RMKTTPLRR |
| 1906. | RMLDRSPsRSAK; |
| | RMLDRSPSRSAK; |
| | RMLDRSPSRsAK |
| 1907. | RMLDRSPsRSAY |
| 1908. | RMLsHISEA |
| 1909. | RMLsHISEV |
| 1910. | RMLsLRDQRL |
| 1911. | RMLsPLSSA |
| 1912. | RMLsPLSSV |
| 1913. | RMLsPSMASK |
| 1914. | RMLsSGVSEI |
| 1915. | RMLsSGVSEV |
| 1916. | RMLsVILIK |
| 1917. | RMPsFLKKNK |
| 1918. | RMPsSTLKK |
| 1919. | RMPsSTLKR |
| 1920. | RMQsTSERL |
| 1921. | RMQsTSERV |
| 1922. | RMRQsPLATK |
| 1923. | RMRQSPLATR |
| 1924. | RMRRsPLLK |
| 1925. | RMRsAGAAQK |
| 1926. | RMRsLSSLREK |
| 1927. | RMRsPPPVSK |
| 1928. | RMRTsPITRK |
| 1929. | RMRTSPITRR |
| 1930. | RMsLLSVV |
| 1931. | RMSSLIRHK |
| 1932. | RMSsPISKK |
| 1933. | RMSsPISKR |
| 1934. | RMSsPLHFV |
| 1935. | RMSsPVLHK |
| 1936. | RMSsRYSQK |
| 1937. | RMSsVKLISK |
| 1938. | RMSsVKLISY |
| 1939. | RMVsLSMRK |
| 1940. | RMVsLSMRY |
| 1941. | RMYKSPLRH |
| 1942. | RMYKsPLRK |
| 1943. | RMYQyIQSK |
| 1944. | RMYQyIQSR |
| 1945. | RMYQyLQSRF |
| 1946. | RMYQyLQSRFK |
| 1947. | RMYQyLQSRK |
| 1948. | RMYsFDDVL |
| 1949. | RMYsGSRSK; RMYSGsRSK; |
| | RMYSGSRsK; RMYsGsRSK; |
| | RMYSGsRsK; RMYsGsRsK |
| 1950. | RMYsGSRSR; RMYSGsRSR; |
| | RMYSGSRsR; RMYsGsRSR; |
| | RMYSGsRsR; RMYsGsRsR |
| 1951. | RMYsKSRDH |
| 1952. | RMYsKSRDK |
| 1953. | RMYsKSRDY |
| 1954. | RMYsPDHRQK |
| 1955. | RMYsPERSK |
| 1956. | RMYsPIIYQA |
| 1957. | RMYsPIPPSL |
| 1958. | RMYsPRNSK |
| 1959. | RMYSPYNHK |
| 1960. | RMYSPYNHR |
| 1961. | RMYsYPRQK |
| 1962. | RMYVTTSTRTYSLG |
| 1963. | RMYVTTSTRTYSLK |
| 1964. | RMYVTTSTRTYSLY |
| 1965. | RNKsYSFIA |
| 1966. | RNLsSPFIF |
| 1967. | RPsSAPDLM; RPSsAPDLM; |
| | RPssAPDLM |
| 1968. | RPsSGFYEL; RPSsGFYEL; |
| | RPssGFYEL |
| 1969. | RPsSLPDL; RPSsLPDL; |
| | RPssLPDL |
| 1970. | RPsSPALYF; RPSSPALYF; |
| | RPssPALYF |
| 1971. | RPsSPIPLL; RPSsPIPLL; |
| | RPssPIPLL |
| 1972. | RPsTPTIDVL; RPStPTIDVL; |
| | RPstPTIDVL |
| 1973. | RPsTPTINV; RPStPTINV; |
| | RPstPTINV |
| 1974. | RPsTPTINVL; RPStPTINVL; |
| | RPstPTINVL |
| 1975. | RPtSPIQIM; RPTsPIQIM; |
| | RPtsPIQIM |
| 1976. | RPAsTGGLSL; |
| | RPAStGGLSL; RPAstGGLSL |
| 1977. | RPAFFsPSL; RPAKsLMSI |
| 1978. | RPAKsMDSF |
| 1979. | RPAKsMDSL |
| 1980. | RPAKsMDSM |
| 1981. | RPAKsMDV |
| 1982. | RPARPsRKGL |
| 1983. | RPAsAGAMF |
| 1984. | RPAsAGAML |
| 1985. | RPAsAGAMM |
| 1986. | RPAsAGAMV |
| 1987. | RPAsARAQPGF |
| 1988. | RPAsARAQPGL |
| 1989. | RPAsARAQPGM |
| 1990. | RPAsARAQPGV |
| 1991. | RPAsEARAPGL |
| 1992. | RPAsPAAKF |
| 1993. | RPAsPAAKL |
| 1994. | RPAsPAAKM |
| 1995. | RPAsPAAKV |
| 1996. | RPAsPALLL |
| 1997. | RPAsPEPEL |
| 1998. | RPAsPGPSL |
| 1999. | RPAsPLMHI |
| 2000. | RPAsPQRAQL |
| 2001. | RPAsPSLQL |
| 2002. | RPAsPSLQLL |
| 2003. | RPAsPtAIRRIGSVTSRQT |
| 2004. | RPAsRFEVL |
| 2005. | RPAsYKKKSML |
| 2006. | RPAtFFPFVA |
| 2007. | RPAtGGPGVA |
| 2008. | RPAtGGPGVF |
| 2009. | RPAtGGPGVL |
| 2010. | RPAtGGPGVM |
| 2011. | RPAtGGPGVV |
| 2012. | RPAtPHLL |
| 2013. | RPAtPTSQF |
| 2014. | RPAtPTSQL |
| 2015. | RPAtPTSQM |
| 2016. | RPAtPTSQV |
| 2017. | RPDsAHKML |
| 2018. | RPDsPTRPTL |
| 2019. | RPDsRLGKTEF |
| 2020. | RPDsRLGKTEL |
| 2021. | RPDsRLGKTEL |
| 2022. | RPDsRLGKTEM |
| 2023. | RPDsRLGKTEV |
| 2024. | RPDsRLLEL |
| 2025. | RPDVAKRLsL |
| 2026. | RPEsDSGLKF |
| 2027. | RPEsDSGLKL |
| 2028. | RPEsDSGLKM |
| 2029. | RPEsDSGLKV |
| 2030. | RPEsKDRKF |
| 2031. | RPEsKDRKL |
| 2032. | RPEsKDRKM |
| 2033. | RPEsKDRKV |
| 2034. | RPEsPAGPF |
| 2035. | RPFHGISTVsL |
| 2036. | RPFsPREAF |
| 2037. | RPFsPREAL |
| 2038. | RPFsPREAM |
| 2039. | RPFsPREAV |
| 2040. | RPGsLERKF |
| 2041. | RPGsLERKL |
| 2042. | RPGsLERKM |
| 2043. | RPGsLERKV |
| 2044. | RPGsRQAGL |
| 2045. | RPHLSGRKLsL |
| 2046. | RPHsPEKAF |
| 2047. | RPHsPEKAL |
| 2048. | RPHsPEKAM |
| 2049. | RPHsPEKAV |
| 2050. | RPHtPTPGI |
| 2051. | RPHtPTPGIYM |
| 2052. | RPIsPGLSF |
| 2053. | RPIsPGLSL |
| 2054. | RPIsPGLSM |
| 2055. | RPIsPGLSV |
| 2056. | RPIsPGLSY |
| 2057. | RPIsPPHTY |
| 2058. | RPIsPRIGAL |
| 2059. | RPIsVIGGVSL |
| 2060. | RPIsVIGGVSLY |
| 2061. | RPItPPRNSA |
| 2062. | RPItPPRNSF |
| 2063. | RPItPPRNSL |
| 2064. | RPItPPRNSM |
| 2065. | RPItPPRNSV |
| 2066. | RPKLHHSLsF |
| 2067. | RPKLSsPAF |
| 2068. | RPKLSsPAL |
| 2069. | RPKLSsPAM |
| 2070. | RPKLSsPAV |
| 2071. | RPKPSSSPVIF; |
| | RPKPSsSPVIF; RPKPsssPVIF; |
| | RPKPsSSPVIF; RPKPSssPVIF; |
| | RPKPsSsPVIF |
| 2072. | RPKPSSsPL |
| 2073. | RPKPSSsPM |
| 2074. | RPKPSSsPV |
| 2075. | RPKPSSsPVI |
| 2076. | RPKsDIVLL |
| 2077. | RPKsNIVLF |
| 2078. | RPKsNIVLL |
| 2079. | RPKsNIVLM |
| 2080. | RPKsNIVLV |
| | RPLsssHEA |
| 2082. | RPKsPLSKM |
| 2083. | RPKsQVAEF |
| 2084. | RPKsQVAEL |
| 2085. | RPKsQVAEM |
| 2086. | RPKsQVAEV |
| 2087. | RPKSSsPIRL |
| 2088. | RPKsVDFDSL |
| 2089. | RPKtPNRASP |
| 2090. | RPKtPPPAP |
| 2091. | RPKtPPVVI |
| 2092. | RPLKPLsPL |
| 2093. | RPLsATRKTL |
| 2094. | RPLsGSGISAF |
| 2095. | RPLsHYSSF |
| 2096. | RPLsKQLSA |
| 2097. | RPLsLIGSTL |
| 2098. | RPLsLLLAL |
| 2099. | RPLsPGALEL |
| 2100. | RPLsPGALQL |
| 2101. | RPLSPGGAF |
| 2102. | RPLsPGGAL |
| 2103. | RPLsPGGAM |
| 2104. | RPLsPGGAV |
| 2105. | RPLsPILHI |
| 2106. | RPLsPLLF |
| 2107. | RPLsPLLL |
| 2108. | RPLsPLLM |
| 2109. | RPLsPLLV |
| 2110. | RPLsPTAFSL |
| 2111. | RPLSSSHEA; RPLSsSHEA; |
| | RPLSSsHEA; RPLssSHEA; |
| | RPLsSsHEA; RPLSssHEA; |
| | RPPtsPGVFGAL |
| 2112. | RPLsVVYVL |
| 2113. | RPLtPRTPA |
| 2114. | RPLTsPESL |
| 2115. | RPMsESPHM |
| 2116. | RPNsPSPTAF |
| 2117. | RPNsPSPTAL |
| 2118. | RPNsPSPTAM |
| 2119. | RPNsPSPTAV |
| 2120. | RPPItQSSL; (Me)RPPItQSSL; |
| | (diMe)RPPItQSSL |
| 2121. | RPPPPPDtPF |
| 2122. | RPPPPPDtPL |
| 2123. | RPPPPPDtPM |
| 2124. | RPPPPPDtPP |
| 2125. | RPPPPPDtPV |
| 2126. | RPPQsSSVSL |
| 2127. | RPPsPGPVF |
| 2128. | RPPsPGPVL |
| 2129. | RPPsPGPVM |
| 2130. | RPPsPGPVV |
| 2131. | RPPsPSSRF |
| 2132. | RPPsPSSRL |
| 2133. | RPPsPSSRM |
| 2134. | RPPsPSSRV |
| 2135. | RPPsSEFLDF |
| 2136. | RPPsSEFLDL |
| 2137. | RPPsSEFLDM |
| 2138. | RPPsSEFLDV |
| 2139. | RPPsSSQQL |
| 2140. | RPPtPTLSL |
| 2141. | RPPtSPGVFGAL; |
| | RPPTsPGVFGAL; |
| | RPRDTRRIsL |
| 2142. | RPPVtKASSF |
| 2143. | RPQKTQsII |
| 2144. | RPQRAtSNVF; |
| | RPQRATsNVF; |
| | RPQRAtsNVF; RPQRATsNVF |
| 2145. | RPQRAtSNVF |
| 2146. | RPQRAtSNVL; |
| | RPQRATsNVL |
| 2147. | RPQRAtSNVM; |
| | RPQRATsNVM |
| 2148. | RPQRAtSNVV; |
| | RPQRATsNVV |
| 2149. | RPQtPKEEA |
| 2150. | RPRsISVEEF; RPRSIsVEEF; |
| | RPRsIsVEEF |
| 2151. | RPRSPSPIS; RPRSPsPIS; |
| | RPRsPsPIS |
| 2152. | RPRsTSQSIVSL; |
| | RPRStSQSIVSL; |
| | RPRSTsQSIVSL; |
| | RPRstSQSIVSL; |
| | RPRsTsQSIVSL; |
| | RPRStsQSIVSL; |
| | RPRstsQSIVSL |
| 2153. | RPRAAtVV |
| 2154. | RPRAAtVVA |
| 2155. | RPRANsGGVDF |
| 2156. | RPRANsGGVDL |
| 2157. | RPRANsGGVDM |
| 2158. | RPRANSGGVDV |
| 2159. | RPRARsVDAL |
| 2160. | RPRDTRRIsL; RPRDtRRISL; |
| 2161. | RPRGPsPLVTM |
| 2162. | RPRGsESLL |
| 2163. | RPRGsQSLF |
| 2164. | RPRGsQSLL |
| 2165. | RPRGsQSLM |
| 2166. | RPRGsQSLV |
| 2167. | RPRHsLNSL |
| 2168. | RPRIPsPIGF |
| 2169. | RPRPGtGLGRVm |
| 2170. | RPRPsSVL; RPRPSsVL; |
| | RPRPssVL |
| 2171. | RPRPASSPAL |
| 2172. | RPRPHsAPSF |
| 2173. | RPRPHsAPSL |
| 2174. | RPRPHsAPSM |
| 2175. | RPRPHsAPSV |
| 2176. | RPRPSsAHVGL |
| 2177. | RPRPsSVL |
| 2178. | RPRPsSVLRTL |
| 2179. | RPRPVsPSSF |
| 2180. | RPRPVsPSSL |
| 2181. | RPRPVsPSSLL |
| 2182. | RPRPVsPSSM |
| 2183. | RPRPVsPSSV |
| 2184. | RPRRsSTQF |
| 2185. | RPRRsSTQL |
| 2186. | RPRRsSTQM |
| 2187. | RPRRsSTQV |
| 2188. | RPRsAVEQL |
| 2189. | RPRsAVLF |
| 2190. | RPRsAVLL |
| 2191. | RPRsAVLM |
| 2192. | RPRsAVLV |
| 2193. | RPRSGsTGSSL; |
| | RPRSGStGSSL; |
| | RPRSGstGSSL; |
| | RPRSGsTGSSL |
| 2194. | RPRsISVEEF; RPRSIsVEEF; |
| | RPRsIsVEEF |
| 2195. | RPRsISVEEM |
| 2196. | RPRsISVEEV |
| 2197. | RPRSLSSPTV; RPRSLSsPTV; |
| | RPRSLssPTV |
| 2198. | RPRSLsSPTVTL; |
| | RPRSLSsPTVTL; |
| | RPRSLssPTVTL |
| 2199. | RPRsLEVTF |
| 2200. | RPRsLEVTI |
| 2201. | RPRSLEVTL |
| 2202. | RPRsLEVTM |
| 2203. | RPRsLEVTV |
| 2204. | RPRSLsSPTV |
| 2205. | RPRSLsSPTVTL; |
| | RPRsLssPTVTL |
| 2206. | RPRSLsSPTVTM |
| 2207. | RPRSLsSPTVTV; |
| | RPRsLssPTVTV |
| 2208. | RPRsMTVSA |
| 2209. | RPRsMVRSF |
| 2210. | RPRsPAARF |
| 2221. | ERPRsPAARL |
| 2212. | RPRsPAARM |
| 2213. | RPRsPAARV |
| 2214. | RPRsPGSNSKV |
| 2215. | RPRsPGSNSKVP |
| 2216. | RPRsPNMQDL |
| 2217. | RPRsPPGGP |
| 2218. | RPRsPPPRAF |
| 2219. | RPRsPPPRAL |
| 2220. | RPRsPPPRAM |
| 2221. | RPRsPPPRAP |
| 2222. | RPRsPPPRAV |
| 2223. | RPRsPPSSP |
| 2224. | RPRsPRENSF |
| 2225. | RPRsPRENSI |
| 2226. | RPRsPRENSL |
| 2227. | RPRsPRENSM |
| 2228. | RPRsPRENSV |
| 2229. | RPRsPRPPP |
| 2230. | RPRsPRQNLI |
| 2231. | RPRsPRQNSF |
| 2232. | RPRsPRQNSI |
| 2233. | RPRsPRQNSM |
| 2234. | RPRsPRQNSV |
| 2235. | RPRsPSPIF |
| 2236. | RPRsPSPIL |
| 2237. | RPRsPSPIM |
| 2238. | RPRsPSPIS; RPRSPsPIS; |
| | RPRsPsPIS |
| 2239. | RPRsPSPIV |
| 2240. | RPRsPSSYDL |
| 2241. | RPRsPTGF |
| 2242. | RPRsPTGL |
| 2243. | RPRsPTGM |
| 2244. | RPRsPTGP |
| 2245. | RPRsPTGPSNSF; |
| | RPRsPTGPsNSF |
| 2246. | RPRsPTGPSNSFL |
| 2247. | RPRsPTGPsNSL |
| 2248. | RPRsPTGPsNSM |
| 2249. | RPRsPTGPsNSV |
| 2250. | RPRsPTGsNSF |
| 2251. | RPRsPTGV |
| 2252. | RPRsPTRSF |
| 2253. | RPRsPTRSL |
| 2254. | RPRsPTRSM |
| 2255. | RPRsPTRSV |
| 2256. | RPRsPWGKL |
| 2257. | RPRsQYNTKL |
| 2258. | RPRTNtPKQL |
| 2259. | RPRtPLRSL |
| 2260. | RPSGRREsF |
| 2261. | RPSGRREsL |
| 2262. | RPSGRREsM |
| 2263. | RPSGRREsV |
| 2264. | RPsLGGRTPL |
| 2265. | RPsNPQL |
| 2266. | RPSRSsPGF |
| 2267. | RPSRSsPGL |
| 2268. | RPSRSsPGM |
| 2269. | RPSRSsPGV |
| 2270. | RPSsGFYEL |
| 2271. | RPsSAPDLM |
| 2272. | RPSsLDAEIDSF |
| 2273. | RPSsLDAEIDSL |
| 2274. | RPSsLDAEIDSM |
| 2275. | RPSsLDAEIDSV |
| 2276. | RPSsLPDF |
| 2277. | RPSsLPDL |
| 2278. | RPSsLPDM |
| 2279. | RPSsLPDV |
| 2280. | RPsSPALYF; RPSsPALYF |
| 2281. | RPsSPALYL |
| 2282. | RPsSPALYM |
| 2283. | RPsSPALYV |
| 2284. | RPSsPRAGAPHAL |
| 2285. | RPSsPRVEDL |
| 2286. | RPSsPSTSw |
| 2287. | RPSsRAVLY |
| 2288. | RPSsRVALMVL |
| 2289. | RPSsVLIEQL |
| 2290. | RPStPGLSV |
| 2291. | RPStPHTITL |
| 2292. | RPStPKSDSEF |
| 2293. | RPStPKSDSEL |
| 2294. | RPStPKSDSEM |
| 2295. | RPStPKSDSEV |
| 2296. | RPStPSRLAL |
| 2297. | RPTKIGRRsL |
| 2298. | RPTsFADEL |
| 2299. | RPTsISWDGL; RPTsISwDGL |
| 2300. | RPTsPIQIM |
| 2301. | RPTsRLNRF |
| 2302. | RPTsRLNRL |
| 2303. | RPTsRLNRLP |
| 2304. | RPTsRLNRM |
| 2305. | RPTsRLNRV |
| 2306. | RPVDPRRRsL |
| 2307. | RPVsPAGPP |
| 2308. | RPVsPAPGA |
| 2309. | RPVsPFQEF |
| 2310. | RPVsPFQEL |
| 2311. | RPVsPFQEM |
| 2312. | RPVsPFQEV |
| 2313. | RPVsPGKDF |
| 2314. | RPVsPGKDI |
| 2315. | RPVsPGKDITA |
| 2316. | RPVsPGKDL |
| 2317. | RPVsPGKDM |
| 2318. | RPVsPGKDV |
| 2319. | RPVsPHSDF |
| 2320. | RPVsPPQKA |
| 2321. | RPVsPSAYm |
| 2322. | RPVsPSSLL |
| 2323. | RPVSPsSLL |
| 2324. | RPVsTDFAQY |
| 2325. | RPVtASITTM |
| 2326. | RPVtPITNF |
| 2327. | RPVtPPRTA |
| 2328. | RPVtPVSDF |
| 2329. | RPVtPVSDL |
| 2330. | RPVtPVSDL |
| 2331. | RPVtPVSDM |
| 2332. | RPVtPVSDV |
| 2333. | RPWsNSRGL |
| 2334. | RPwsNSRGL |
| 2335. | RPWsPAVSA |
| 2336. | RPwsPAVSA |
| 2337. | RPWsPAVSF |
| 2338. | RPWsPAVSL |
| 2339. | RPWsPAVSM |
| 2340. | RPWsPAVSV |
| 2341. | RPWsPPPTGSL |
| 2342. | RPYPsPGAVL |
| 2343. | RPYsPPFF |
| 2344. | RPYsPPFFSF |
| 2345. | RPYsPPFFSL |
| 2346. | RPYsPPFFSM |
| 2347. | RPYsPPFFSV |
| 2348. | RPYsPSEYAL |
| 2349. | RPYsPSQYAL |
| 2350. | RPYsQVNVL |
| 2351. | RPYtNKVITL |
| 2352. | RQAsIELPSM |
| 2353. | RQAsIELPSMAV |
| 2354. | RQAsIELPSMAVA |
| 2355. | RQAsIELPSV |
| 2356. | RQAsLSISV |
| 2357. | RQAsPLVHK |
| 2358. | RQAsPLVHR |
| 2359. | RQAsPLVHY |
| 2360. | RQAsPPRRL |
| 2361. | RQDsTPGKVFL |
| 2362. | RQDStPGKVFL |
| 2363. | RQDsTPGKVFV |
| 2364. | RQFMRRTsL |
| 2365. | RQIsTSGEL |
| 2366. | RQIStSGEL |
| 2367. | RQIstSGEL |
| 2368. | RQIsFKAEV |
| 2369. | RQIsQDVKL |
| 2370. | RQIsQDVKV |
| 2371. | RQKsPLFQF |
| 2372. | RQLsALHRA |
| 2373. | QLsLEGSGLGV |
| 2374. | RQLsSGVSEI |
| 2375. | RQLsSGVSEV |
| 2376. | RQMsGAQIKI |
| 2377. | RQMsRFKEA |
| 2378. | RQPsEEEII |
| 2379. | RQPsEEEIIKL |
| 2380. | RQPsIELPSM |
| 2381. | RQPsLAKRV |
| 2382. | RQPsLKRSL |
| 2383. | RQSsFEPEF |
| 2384. | RQSsSRFNL |
| 2385. | RQYsVTDAL |
| 2386. | RRsSIQSTF; RRSsIQSTF; |
| | RRssIQSTF |
| 2387. | RRsSQSWSL; RRSsQSwSL |
| 2388. | RRSsIQSTF; RRssIQSTF |
| 2389. | RRsSYLLAI; RRSsYLLAI; |
| | RRssYLLAI |
| 2390. | RRAsFAKSF; RRASFAKsF; |
| | RRAsFAKsF |
| 2391. | RRAsFAKSK; RRASFAKsK; |
| | RRASFAKsK |
| 2392. | RRAsFAKSL; RRASFAKsL; |
| | RRAsFAKsL |
| 2393. | RRAsFAKSM; RRASFAKsM; |
| | RRAsFAKsM |
| 2394. | RRAsFAKSR; RRASFAKsR; |
| | RRASFAKsR |
| 2395. | RRAsIITKY |
| 2396. | RRAsLSEIGF |
| 2397. | RRAsLSEIGK |
| 2398. | RRAsLSEIGY |
| 2399. | RRAsLSYSF |
| 2400. | RRAsQEANL |
| 2401. | RRAsSPFRF |
| 2402. | RRAsSPFRK |
| 2403. | RRAsSPFRL |
| 2404. | RRAsSPFRM |
| 2405. | RRAsSPFRR |
| 2406. | RRAsVFVKF |
| 2407. | RRAsVFVKK |
| 2408. | RRAsVFVKL |
| 2409. | RRAsVFVKM |
| 2410. | RRAsVFVKR |
| 241I. | RRDsIVAEF |
| 2412. | RRDsIVAEK |
| 2413. | RRDsIVAEL |
| 2414. | RRDsIVAER |
| 2415. | RRDsIVAEY |
| 2416. | RRDsLQKPGL |
| 2417. | RRFsTEYEL; RRFStEYEL; |
| | RRFstEYEL |
| 2418. | RRFsDFLGL |
| 2419. | RRFsFKF |
| 2420. | RRFsFKK |
| 2421. | RRFsFKKSF |
| 2422. | RRFsFKKSK |
| 2423. | RRFsFKKSL |
| 2424. | RRFsFKKSM |
| 2425. | RRFsFKKSR |
| 2426. | RRFsFKL |
| 2427. | RRFsFKM |
| 2428. | RRFsFKR |
| 2429. | RRFsFSGNTL |
| 2430. | RRFsGLLN |
| 2431. | RRFsGLLNC; RRFsGLLNc |
| 2432. | RRFsGTAVY |
| 2433. | RRFsGTVRF |
| 2434. | RRFsGTVRK |
| 2435. | RRFsGTVRL |
| 2436. | RRFsGTVRM |
| 2437. | RRFsGTVRR |
| 2438. | RRFsIATLR |
| 2439. | RRFSLSPSL |
| 2440. | RRFSLTTLR |
| 2441. | RRFSLTTLRNF |
| 2442. | RRFSLTTLRNY |
| 2443. | RRFSPDDKYSF |
| 2444. | RRFSPDDKYSK |
| 2445. | RRFSPDDKYSL |
| 2446. | RRFPDDKYSM |
| 2447. | RRFSPDDKYSR |
| 2448. | RRFSPPRRF |
| 2449. | RRFSPPRRK |
| 2450. | RRFSPPRRL |
| 2451. | RRFSPPRRM |
| 2452. | RRFSPPRRML |
| 2453. | RRFSPPRRR |
| 2454. | RRFSPPRRY |
| 2455. | RRFSRLENRY |
| 2456. | RRFSRSDEL |
| 2457. | RRFSRSPIF; RRFsRsPIF; |
| | RRFsRSPIK |
| 2458. | RRFsRsPIK |
| 2459. | RRFsRSPIL; RRFsRsPIL |
| 2460. | RRFSRSPIM |
| 2461. | RRFSRsPIR; RRFsRSPIR; |
| | RRFsRsPIR |
| 2462. | RRFsRSPIRF; RRFsRsPIRF; |
| | RRFsRSPIRK |
| 2463. | RRFsRsPIRK |
| 2464. | RRFsRSPIRL; RRFsRsPIRL |
| 2465. | RRFsRsPIRR; RRFsRSPIRR |
| 2466. | RRFsRSPIRY:RRFsRsPIRY |
| 2467. | RRFsRSPIY; RRFsRsPIY |
| 2468. | RRFsRSPK |
| 2469. | RRFsSSDFSDL |
| 2470. | RRFsSYSQM |
| 2471. | RRFsVSTLR |
| 2472. | RRFsVSTLRNL |
| 2473. | RRFsVSTLRNLGL |
| 2474. | RRFsVSTLRNLGLG |
| 2475. | RRFsVSTLRNLGLGK |
| 2476. | RRFsVTLRL |
| 2477. | RRFsVTTMR |
| 2478. | RRFtEIYEF |
| 2479. | RRFtPPSPAF |
| 2480. | RRFtPPSPAK |
| 2481. | RRFtPPSPAR |
| 2482. | RRFtPPSPAY |
| 2483. | RRGsFEVTL |
| 2484. | RRGsFEVTLL |
| 2485. | RRGsFPLAA |
| 2486. | RRGsGPEIF |
| 2487. | RRGsGPEIFT |
| 2488. | RRGsGPEIFTF |
| 2489. | RRGsLLGSM |
| 2490. | RRGsLTLTI |
| 2491. | RRGsNVALM |
| 2492. | RRGsPVRQL |
| 2493. | RRGsYPFIDF |
| 2494. | RRHsASNLHAL |
| 2495. | RRHsLENKV |
| 2496. | RRIDIsPSTF |
| 2497. | RRIDIsPSTFRK |
| 2498. | RRIDIsPSTK |
| 2499. | RRIDIsPSTL |
| 2500. | RRIDIsPSTLR |
| 2501. | RRIDIsPSTLRK |
| 2502. | RRIDIsPSTR |
| 2503. | RRIDIsPSTY |
| 2504. | RRIsDPEVF |
| 2505. | RRIsDPQVF |
| 2506. | RRISGVDRF |
| 2507. | RRISGVDRK |
| 2508. | RRISGVDRL |
| 2509. | RRISGVDRM |
| 2510. | RRIsGVDRR |
| 2511. | RRIsGVDRY |
| 2512. | RRIsGVDRYF |
| 2513. | RRIsGVDRYK |
| 2514. | RRIsGVDRYK |
| 2515. | RRIsGVDRYL |
| 2516. | RRIsGVDRYR |
| 2517. | RRIsGVDRYY |
| 2518. | RRIsIGSLF |
| 2519. | RRIsLTKRL |
| 2520. | RRIsQIQQL |
| 2521. | RRIsVFKYV |
| 2522. | RRIsVTSKV |
| 2523. | RRKsDDVHL |
| 2524. | RRKsLVLKF |
| 2525. | RRKsPPPSF |
| 2526. | RRKsPPPSK |
| 2527. | RRKsPPPSL |
| 2528. | RRKsPPPSM |
| 2529. | RRKsPPPSR |
| 2530. | RRKsQLDSF |
| 2531. | RRKsQLDSK |
| 2532. | RRKsQLDSL |
| 2533. | RRKSQLDSM |
| 2534. | RRKsQLDSR |
| 2535. | RRKsQLDSY |
| 2536. | RRKsQVAEF |
| 2537. | RRKsQVAEK |
| 2538. | RRKsQVAEL |
| 2539. | RRKsQVAEM |
| 2540. | RRKsQVAER |
| 2541. | RRKsQVAEV |
| 2542. | RRKsQVAEY |
| 2543. | RRLGSPHRF |
| 2544. | RRLGSPHRK |
| 2545. | RRLGSPHRL |
| 2546. | RRLGSPHRM |
| 2547. | RRLGSPHRR |
| 2548. | RRLsAARLL |
| 2549. | RRLsADIRF |
| 2550. | RRLsADIRK |
| 2551. | RRLsADIRL |
| 2552. | RRLSADIRM |
| 2553. | RRLsADIRR |
| 2554. | RRLsADIRY |
| 2555. | RRLsDSPVF |
| 2556. | RRLsELLRY |
| 2557. | RRLsERETR |
| 2558. | RRLsESSAL |
| 2559. | RRLsFLVSF |
| 2560. | RRLsFLVSK |
| 2561. | RRLsFLVSL |
| 2562. | RRLSFLVSM |
| 2563. | RRLsFLVSR |
| 2564. | RRLsFLVSY |
| 2565. | RRLsFQAEY |
| 2566. | RRLsGELISM |
| 2567. | RRLsGGSHSF |
| 2568. | RRLSGGSHSK |
| 2569. | RRLsGGSHSL |
| 2570. | RRLSGGSHSM |
| 2571. | RRLSGGSHSR |
| 2572. | RRLsGGSHSY |
| 2573. | RRLSGPLHTF |
| 2574. | RRLSGPLHTK |
| 2575. | RRLSGPLHTL |
| 2576. | RRLSGPLHTM |
| 2577. | RRLSGPLHTR |
| 2578. | RRLSGPLHTV |
| 2579. | RRLSLFLNV |
| 2580. | RRLsLFLVL |
| 2581. | RRLsLPGLL |
| 2582. | RRLSLSRSL |
| 2583. | RRLSNLPTF |
| 2584. | RRLSNLPTK |
| 2585. | RRLSNLPTR |
| 2586. | RRLSNLPTV |
| 2587. | RRLSNLPTY |
| 2588. | RRLSPAPQF |
| 2589. | RRLSPAPQK |
| 2590. | RRLSPAPQL |
| 2591. | RRLSPAPQM |
| 2592. | RRLSPAPQR |
| 2593. | RRLSPKASQVF |
| 2594. | RRLSPKASQVK |
| 2595. | RRLSPKASQVL |
| 2596. | RRLSPKASQVM |
| 2597. | RRLSPKASQVR |
| 2598. | RRLSPVPVPF |
| 2599. | RRLSPVPVPK |
| 2600. | RRLSPVPVPL |
| 2601. | RRLSPVPVPM |
| 2602. | RRLSPVPVPR |
| 2603. | RRLSRELQF |
| 2604. | RRLSRELQK |
| 2605. | RRLSRELQL |
| 2606. | RRLSRELQM |
| 2607. | RRLSRELQR |
| 2608. | RRLsRKL |
| 2609. | RRLsRKLSL |
| 2610. | RRLsSQFEN |
| 261I. | RRLsVEIYDKF |
| 2612. | RRLSVERIF |
| 2613. | RRLSVERIK |
| 2614. | RRLSVERIL |
| 2615. | RRLSVERIM |
| 2616. | RRLSVERIR |
| 2617. | RRLsYVLFI |
| 2618. | RRLTHLSF |
| 2619. | RRLTHLSK |
| 2620. | RRLTHLSL |
| 2621. | RRLTHLSM |
| 2622. | RRLTHLSR |
| 2623. | RRLtLHSVF |
| 2624. | RRMsFQKP |
| 2625. | RRMsFSGIFR |
| 2626. | RRMsLLSVF |
| 2627. | RRMsLLSVK |
| 2628. | RRMsLLSVL |
| 2629. | RRMsLLSVM |
| 2630. | RRMsLLSVR |
| 2631. | RRMsLLSVV |
| 2632. | RRMsLLSVY |
| 2633. | RRMSPKAQF |
| 2634. | RRMSPKAQK |
| 2635. | RRMSPKAQL |
| 2636. | RRMSPKAQM |
| 2637. | RRMSPKAQR |
| 2638. | RRMSPKPF |
| 2639. | RRMSPKPK |
| 2640. | RRMSPKPL |
| 2641. | RRMSPKPM |
| 2642. | RRMSPKPR |
| 2643. | RRMsVAEQVDY |
| 2644. | RRMsVGDRAG |
| 2645. | RRNsAPVSV |
| 2646. | RRNsFIGTPY |
| 2647. | RRNSINRNF |
| 2648. | RRNsISLREL |
| 2649. | RRNsKIFLDL |
| 2650. | RRNsLLHGY |
| 2651. | RRNSNPVIAEF |
| 2652. | RRNSNPVIAEK |
| 2653. | RRNSNPVIAEL |
| 2654. | RRNSNPVIAEM |
| 2655. | RRNSNPVIAER |
| 2656. | RRNsSERTF |
| 2657. | RRNSSERTK |
| 2658. | RRNsSERTL |
| 2659. | RRNsSERTM |
| 2660. | RRNSSERTR |
| 2661. | RRNSSERTY |
| 2662. | RRNsSIVGF |
| 2663. | RRNsSIVGK |
| 2664. | RRNSSIVGL |
| 2665. | RRNSSIVGM |
| 2666. | RRNsSIVGR |
| 2667. | RRNsSIVGY |
| 2668. | RRNsVFQQGF |
| 2669. | RRNsVFQQGK |
| 2670. | RRNSVFQQGL |
| 2671. | RRNsVFQQGM |
| 2672. | RRNsVFQQGR |
| 2673. | RRNsVFQQGY |
| 2674. | RRPKtLRL |
| 2675. | RRPsHEGYL |
| 2676. | RRPsIAPVL |
| 2677. | RRPsKPRLI |
| 2678. | RRPsLLSEF |
| 2679. | RRPsLQGNTL |
| 2680. | RRPsLVHGF |
| 2681. | RRPsLVHGK |
| 2682. | RRPSLVHGL |
| 2683. | RRPSLVHGM |
| 2684. | RRPsLVHGR |
| 2685. | RRPsLVHGY |
| 2686. | RRPsQNAISF |
| 2687. | RRPsQNAISFF |
| 2688. | RRPsQPYMF |
| 2689. | RRPsRPHMF |
| 2690. | RRPsRPHMFP |
| 2691. | RRPsVFERF |
| 2692. | RRPsVFERK |
| 2693. | RRPsVFERL |
| 2694. | RRPSVFERM |
| 2695. | RRPsVFERR |
| 2696. | RRPsVFERY |
| 2697. | RRPsYRKIF |
| 2698. | RRPsYRKIK |
| 2699. | RRPsYRKIL |
| 2700. | RRPSYRKIM |
| 2701. | RRPsYRKIR |
| 2702. | RRPsYRKIY |
| 2703. | RRPsYTLGF |
| 2704. | RRPsYTLGK |
| 2705. | RRPsYTLGL |
| 2706. | RRPsYTLGM |
| 2707. | RRPsYTLGR |
| 2708. | RRPsYTLGV |
| 2709. | RRPsYTLGY |
| 2710. | RRQsFAVLR |
| 2711. | RRQsKVEAL |
| 2712. | RRREDsYHV |
| 2713. | RRRsAPPEL |
| 2714. | RRRsAVHML |
| 2715. | RRRsLERLL |
| 2716. | RRSFsLE |
| 2717. | RRSsDIISL |
| 2718. | RRSsFLQ |
| 2719. | RRssFLQLF; RRSsFLQVF |
| 2720. | RRSSFLQVK |
| 2721. | RRSsFLQVL |
| 2722. | RRsSFLQVM; RRSSFLQVM; |
| | RRssFLQVM |
| 2723. | RRSSFLQVR |
| 2724. | RRssFLQVV |
| 2725. | RRSsFLQVY |
| 2726. | RRSsIGLRF |
| 2727. | RRSsIGLRK |
| 2728. | RRSsIGLRL |
| 2729. | RRSSIGLRM |
| | RRssVDLGF |
| 2730. | RRSsIGLRR |
| 2731. | RRSsIGLRV |
| 2732. | RRSsIGLRY |
| 2733. | RRsSIPITV; RRSsIPITV |
| 2734. | RRSsIQSTF; RRsSIQSTF; |
| | RRssIQSTF |
| 2735. | RRSsIQSTK |
| 2736. | RRSsIQSTL |
| 2737. | RRSsIQSTM |
| 2738. | RRSsIQSTR |
| 2739. | RRSsIQSTY |
| 2740. | RRSsISSWL |
| 2741. | RRSsLDAEIDSF |
| 2742. | RRSsLDAEIDSL |
| 2743. | RRSsLDAEIDSM |
| 2744. | RRSsLDAEIDSV |
| 2745. | RRSsLLSLM |
| 2746. | RRSsQSWSF; RRsSQSWSF; |
| | RRssQSWSF |
| 2747. | RRSsQSWSK |
| 2748. | RRSsQSWSL; RRSsQSwSL; |
| | RRsSQSWSL |
| 2749. | RRsSQSWSM |
| 2750. | RRSsQSWSR |
| 2751. | RRsSQSWSV |
| 2752. | RRSsQSWSY |
| 2753. | RRSsSVAQV |
| 2754. | RRSSTASLVKF |
| 2755. | RRSSTASLVKK |
| 2756. | RRSSTASLVKL |
| 2757. | RRSSTASLVKM |
| 2758. | RRSSTASLVKR |
| 2759. | RRSsVDLGF; RRsSVDLGF; |
| 2760. | RRSsVDLGK; RRsSVDLGK; |
| | RRssVDLGK |
| 2761. | RRSsVDLGL; RRsSVDLGL; |
| | RRssVDLGL |
| 2762. | RRSSVDLGM |
| 2763. | RRSsVDLGR; RRsSVDLGR; |
| | RRssVDLGR |
| 2764. | RRSsVDLGY; RRsSVDLGYI |
| | RRssVDLGY |
| 2765. | RRSsVKVEA |
| 2766. | RRSsVKVEF |
| 2767. | RRSsVKVEK |
| 2768. | RRSsVKVEL |
| 2769. | RRSSVKVEM |
| 2770. | RRSsVKVER |
| 2771. | RRSsVKVEY |
| 2772. | RRTSPITRF |
| 2773. | RRTSPITRK |
| 2774. | RRTSPITRL |
| 2775. | RRTSPITRM |
| 2776. | RRTSPITRR |
| 2777. | RRVsIGVQL |
| 2778. | RRVsPLNL |
| 2779. | RRVsPLNLSSVTP |
| 2780. | RRVsSNGIFDL |
| 2781. | RRVVQRSSF |
| 2782. | RRVVQRSsK |
| 2783. | RRVVQRSsL |
| 2784. | RRVVQRSSM |
| 2785. | RRVVQRSsR |
| 2786. | RRVVQRSsY |
| 2787. | RRYsASTVDVIEM |
| 2788. | RRYsDLTTL |
| 2789. | RRYsDPPTY |
| 2790. | RRYSGKTEF |
| 2791. | RRYSGKTEK |
| 2792. | RRYSGKTEL |
| 2793. | RRYSGKTER |
| 2794. | RRYSGKTEY |
| 2795. | RRYSGNMEF |
| 2796. | RRYSGNMEK |
| 2797. | RRYSGNMEL |
| 2798. | RRYSGNMEM |
| 2799. | RRYSGNMER |
| 2800. | RRYsKFFDL |
| 2801. | RRYsLPLKSIYM |
| 2802. | RRYsPPIER |
| 2803. | RRYsPPIQ |
| 2804. | RRYsPPIQF |
| 2805. | RRYsPPIQK |
| 2806. | RRYSPPIQL |
| 2807. | RRYSPPIQM |
| 2808. | RRYsPPIQR |
| 2809. | RRYsPPIQY |
| 2810. | RRYSRSPYSF |
| 2811. | RRYSRSPYSK |
| 2812. | RRYSRSPYSL |
| 2813. | RRYSRSPYSM |
| 2814. | RRYSRSPYSR |
| 2815. | RRYTNRVVTF |
| 2816. | RRYTNRVVTK |
| 2817. | RRYTNRVVTL |
| 2818. | RRYTNRVVTM |
| 2819. | RRYTNRVVTR |
| 2820. | RSAsFSRKV |
| 2821. | RSAsLAKL |
| 2822. | RSAsLAKLGY |
| 2823. | RSAsPDDDLGSSN |
| 2824. | RSAsPSSQGW |
| 2825. | RSAsPSSQGw |
| 2826. | RSAsPTVPR |
| 2827. | RSAsQERSL |
| 2828. | RSAsSATQVHK |
| 2829. | RSAsSATQVHY |
| 2830. | RSAsVGAEEY |
| 2831. | RSDSSQPML |
| 2832. | RSDSsQPML |
| 2833. | RSDssQPML |
| 2834. | RSDPSKsPGSLRY |
| 2835. | RSDsPKIDL |
| 2836. | RSDsPKIDY |
| 2837. | RSDsRAQAV |
| 2838. | RSDsRAQAY |
| 2839. | RSDsVGENL |
| 2840. | RSDsVGENY |
| 2841. | RSDsYVEL |
| 2842. | RSDsYVELSQY |
| 2843. | RSEPSKsPGSLRY |
| 2844. | RSEsKDRKF |
| 2845. | RSEsKDRKL |
| 2846. | RSEsKDRKM |
| 2847. | RSEsKDRKV |
| 2848. | RSEsPKIDL |
| 2849. | RSEsPKIDY |
| 2850. | RSEsPPAEL |
| 2851. | RSEsRAQAV |
| 2852. | RSEsRAQAY |
| 2853. | RSEsTENQSY |
| 2854. | RSEsVGENL |
| 2855. | RSEsVGENY |
| 2856. | RSEsYVELSQY |
| 2857. | RSFsGLIKR |
| 2858. | RSFsPTMKV |
| 2859. | RSFsVEREL |
| 2860. | RSFtPLSI |
| 2861. | RSFPLSILK |
| 2862. | RSGsLERKF |
| 2863. | RSGsLERKL |
| 2864. | RSGsLERKM |
| 2865. | RSGsLERKV |
| 2866. | RSHsPLRSK |
| 2867. | RSHsPMSNR |
| 2868. | RSHsPPLKL |
| 2869. | RSHSsPASL |
| 2870. | RSHsSPASL |
| 2871. | RSHSsPASL |
| 2872. | RSIsASDLTF |
| 2873. | RSIsNEGLTL |
| 2874. | RSIsSLLRF |
| 2875. | RSIsTPTCL; RSIsTPTcL; |
| | RSIsTPTc |
| 2876. | RSIsVGENL |
| 2877. | RSKsATLLY |
| 2878. | RSKsLTNLV |
| 2879. | RSKtPPKSY |
| 2880. | RSLsSGESL; RSLSsGESL; |
| | RSLssGESL |
| 2881. | RSLGsVQAPSY |
| 2882. | RSLsASPAL |
| 2883. | RSLsERLLQL |
| 2884. | RSLsESYEL |
| 2885. | RSLsFSDEM |
| 2886. | RSLsPFRRHSW; |
| | RSLsPFRRHsW; |
| | RSLsPFRRHsW |
| 2887. | RSLsPGGAA |
| 2888. | RSLsPGGAALGY |
| 2889. | RSLsPGGAF |
| 2890. | RSLsPGGAL |
| 2891. | RSLsPGGAM |
| 2892. | RSLsPGGAV |
| 2893. | RSLsPILPGR |
| 2894. | RSLsPLIKF |
| 2895. | RSLsPLLF |
| 2896. | RSLsPLLL |
| 2897. | RSLsPLLM |
| 2898. | RSLsPLLV |
| 2899. | RSLsPSSNSAF |
| 2900. | RSLsQELVGV |
| 2901. | RSLsRVRVL |
| 2902. | RSLsSYRGKY |
| 2903. | RSLsTTNVF |
| 2904. | RSLsVEIVK |
| 2905. | RSLsVEIVY |
| 2906. | RSLsVGSEF |
| 2907. | RSLsVPVDL |
| 2908. | RSLTHLsL |
| 2909. | RSLtHPPTI |
| 2910. | RSMsGGHGL |
| 2911. | RSMsMPVAH |
| 2912. | RSMsMPVAK |
| 2913. | RSNsLVSTF |
| 2914. | RSNsPLPSI |
| 2915. | RsPEDEYELLMPHRISSH |
| | RssSFVLPKL; RsSsFVLPKL; |
| | RSSSFVLPKL; RSSSFVLPKL; |
| | RSSSFVLPKL |
| 2916. | RsPEPDPYLSY |
| 2917. | RSPsFNMQL |
| 2918. | RSPsKPTLAY |
| 2919. | RSPsPKTSL |
| 2920. | RSPsPSFRWPF |
| 2921. | RSPsPTLSYY |
| 2922. | RsPTKSSLDY |
| 2923. | RsPTKSSLDYR |
| 2924. | RSRPALsPL |
| 2925. | RSRRsPLLK |
| 2926. | RSRRsPLLY |
| 2927. | RSRsPLEL |
| 2928. | RSRsPLGFY |
| 2929. | RSRsPPPVS |
| 2930. | RSRsPPPVSK |
| 2931. | RSRsPPPVSY |
| 2932. | RSRsPRPAF |
| 2933. | RSRsPRPAL |
| 2934. | RSRsPRPAM |
| 2935. | RSRsPRPAV |
| 2936. | RSRsRDRMY |
| 2937. | RSRsVPVSF |
| 2938. | RSRsYsPRRY |
| 2939. | RSRsYTPEY |
| 2940. | RSRTsPITRR |
| 2941. | RSRTsPITRY |
| 2942. | RsSFLQVF |
| 2943. | RSSPRTIsF |
| 2944. | RSSQFGsLEF |
| 2945. | RSSsAPLGL |
| 2946. | RSSsFKDFAK |
| 2947. | RSSsFSDTL |
| 2948. | RsSSFVLPKL; RSsSFVLPKL; |
| 2949. | RSSSLIRHK |
| 2950. | RSSSLIRHY |
| 2951. | RSSsLQRRV |
| 2952. | RsSSLSDFSW; |
| | RSsSLSDFSW; |
| | RSSsLSDFSW; RssSLSDFSW; |
| | RsSsLSDFSW; RSssLSDFSW; |
| | RsssLSDFSW |
| 2953. | RsSSPFLSK; RSsSPFLSK; |
| | RSSsPFLSK; RssSPFLSK; |
| | RsSsPFLSK; RSssPFLSK; |
| | RsssPFLSK |
| 2954. | RSSsPLQL |
| 2955. | RSSsPPILTK |
| 2956. | RSSsPVTEL |
| 2957. | RSStPLPTI |
| 2958. | RSTsLSLKY |
| 2959. | RSVsGFLHF |
| 2960. | RSVsLDSQM |
| 2961. | RSVsLDSQMGY |
| 2962. | RSVsLSMRK |
| 2963. | RSVsLSMRY |
| 2964. | RSVsPTFL |
| 2965. | RSVsPVQDL |
| 2966. | RsWKYNQSISLRRP |
| 2967. | RSWsPPPEV |
| 2968. | RSWsPPPEVSR |
| 2969. | RSYsDPPLKF |
| 2970. | RSYsGSRsK |
| 2971. | RSYGSRSR |
| 2972. | RSYSGSRSR |
| 2973. | RSYsGSRsY |
| 2974. | RSYsPDHRQK |
| 2975. | RSYsPDHRQY |
| 2976. | RSYsPERSK |
| 2977. | RSYsPERSKSY |
| 2978. | RSYsPERSKSYSF |
| 2979. | RSYsPERSY |
| 2980. | RSYsPRNSR |
| 2981. | RSYsPRNSY |
| 2982. | RSYSRsFSK |
| 2983. | RSYsRSFSR |
| 2984. | RSYSRsFSY |
| 2985. | RSYsYPRQK |
| 2986. | RSYsYPRQY |
| 2987. | RSYVTTSTRTYsLG |
| 2988. | RTsSFALNL; RTSsFALNL; |
| | RTssFALNL |
| 2989. | RTAsFAVRK |
| 2990. | RTAsFAVRY |
| 2991. | RTASLIIKV |
| 2992. | RTAsLSNQEcQLY |
| 2993. | RTAsLVSGL |
| 2994. | RTAsPPALPK |
| 2995. | RTAsPPPPPK |
| 2996. | RTAtADDKKLQF |
| 2997. | RTDPSKsPGSLRY |
| 2998. | RTDsIGEKL |
| 2999. | RTDsIGEKLGRY |
| 3000. | RTDsPKIDL |
| 3001. | RTDsPKIDY |
| 3002. | RTDsRAQAV |
| 3003. | RTDsRAQAY |
| 3004. | RTDsREQKL |
| 3005. | RTDSRGVNL |
| 3006. | RTDsYVELSQY |
| 3007. | RTEPSKsPGSLRY |
| 3008. | RTEsDSGLKF |
| 3009. | RTEsDSGLKK |
| 3010. | RTEsDSGLKL |
| 3011. | RTEsDSGLKM |
| 3012. | RTEsDSGLKV |
| 3013. | RTEsPKIDL |
| 3014. | RTEsPKIDY |
| 3015. | RTEsRAQAV |
| 3016. | RTEsRAQAY |
| 3017. | RTEsYVELSQY |
| 3018. | RTFsDESNVL |
| 3019. | RTFsESSVW |
| 3020. | RTFsLDTIL |
| 3021. | RTFsPTY |
| 3022. | RTFsPTYGF |
| 3023. | RTFsPTYGL |
| 3024. | RTFsPTYGLLR |
| 3025. | RTFsPTYGM |
| 3026. | RTFsPTYGV |
| 3027. | RTFsYIKNK |
| 3028. | RTGsPALGL |
| 3029. | RTHsLLLLL |
| 3030. | RTIsAQDTLAY |
| 3031. | RTIsNPEVVMK |
| 3032. | RTIsPPTLGTL |
| 3033. | RTIsQSSSL |
| 3034. | RtISVILFL; RTIsVILFL; |
| | RtIsVILFL |
| 3035. | RTLsHISEA |
| 3036. | RTLsHISEV |
| 3037. | RTLsMDKGF |
| 3038. | RTLsPEIITV |
| 3039. | RTLsPSSGY |
| 3040. | RTLsVESLI |
| 3041. | RTMsEAALVRK |
| 3042. | RTMsPIQVL |
| 3043. | RTNsPGFQK |
| 3044. | RTPsDVKEL |
| 3045. | RTPsFLKKNK |
| 3046. | RTPsFLKKNY |
| 3047. | RTPsISFHH |
| 3048. | RTPsPARPAL |
| 3049. | RTPsPKSLPSYL |
| 3050. | RTPsQIIRK |
| 3051. | RTPsSSSTLAY |
| 3052. | RTRsLPITI |
| 3053. | RTRsLSSLREK |
| 3054. | RTRsLSSLREY |
| 3055. | RTRsPSPTF |
| 3056. | RTRsPSPTL |
| 3057. | RTRsPSPTM |
| 3058. | RTRsPSPTV |
| 3059. | RTSsFALNL |
| 3060. | RTSsFTEQL |
| 3061. | RTSSFtFQN; RTSsFTFQN; |
| | RTSsFtFQN |
| 3062. | RTSsPLFNK |
| 3063. | RTSsQRSTLTY |
| 3064. | RTVsPAHVL |
| 3065. | RTVsPELIL |
| 3066. | RTYKsPLRH |
| 3067. | RTYKsPLRK |
| 3068. | RTYKsPLRY |
| 3069. | RTYSGPMNK |
| 3070. | RTYSGPMNKV |
| 3071. | RTYsHGTYR |
| 3072. | RTYsLGSAL |
| 3073. | RVAsFAVRK |
| 3074. | RVAsFAVRY |
| 3075. | RVAsPLVHK |
| 3076. | RVAsPLVHY |
| 3077. | RVAsPPPPPK |
| 3078. | RVAsPPPPPY |
| 3079. | RVAsPSRKV |
| 3080. | RVAsPTSGV |
| 3081. | RVAsPTSGVK |
| 3082. | RVAsPTSGVKK |
| 3083. | RVAsPTSGVKR |
| 3084. | RVAsPTSGVY |
| 3085. | RVAsWAVSF |
| 3086. | RVDsLEFSL |
| 3087. | RVDsPSHGL |
| 3088. | RVDsPVTV |
| 3089. | RVDsTTcLF |
| 3090. | RVGsLVLNL |
| 3091. | RVISGVLQL |
| 3092. | RVKLPsGSKK |
| 3093. | RVKsPGsGHVK |
| 3094. | RVKsPGsGHVY |
| 3095. | RVKsPISLK |
| 3096. | RVKsPSPKSER |
| 3097. | RVKsPSPKSEY |
| 3098. | RVKsWADNL |
| 3099. | RVKtPTSQSYK |
| 3100. | RVKtPTSQSYR |
| 3101. | RVKtPTSQSYY |
| 3102. | RVKTtPLRR |
| 3103. | RVKTtPLRY |
| 3104. | RVKVDGPRSPsY |
| 3105. | RVLDRSPsRSAK |
| 3106. | RVLDRSPsRSAY |
| 3107. | RVLHsPPAV |
| 3108. | RVLsGVVTK |
| 3109. | RVLsPLIIK |
| 31I0. | RVMsSPSAMK |
| 311I. | RVMSsPSAMK |
| 3112. | RVMssPSAMK |
| 3113. | RVPsINQKI |
| 3114. | RVPsKsLDL; RVPsKSLDL; |
| | RVPSKsLDL |
| 3115. | RVPsLLVLL |
| 3116. | RVPsPTPAPK |
| 3117. | RVPsSTLKK |
| 3118. | RVPsSTLKY |
| 3119. | RVRKLPsTTL |
| 3120. | RVRQsPLATK |
| 3121. | RVRQsPLATR |
| 3122. | RVRQsPLATY |
| 3123. | RVRRsSFLNAK; |
| | RVRRSSFLNAK; |
| | RVRRssFLNAK; |
| | RVRRSSFLNAK |
| 3124. | RVRsLSSLREK |
| 3125. | RVRsLSSLREY |
| 3126. | RVRsPTRSF |
| 3127. | RVRsPTRSL |
| 3128. | RVRsPTRSM |
| 3129. | RVRsPTRSP |
| 3130. | RVRsPTRSV |
| 3131. | RVSSLTLHL |
| 3132. | RVSsPISKK |
| 3133. | RVSsPISKY |
| 3134. | RVSsPLASF |
| 3135. | RVSsRFSSK |
| 3136. | RVSsRFSSR |
| 3137. | RVSsRFSSY |
| 3138. | RVSsVKLISK |
| 3139. | RVSsVKLISY |
| 3140. | RVTsAEIKL |
| 3141. | RVVPsPLQF |
| 3142. | RVVsLSMRK |
| 3143. | RVVsLSMRY |
| 3144. | RVVsPGIDL |
| 3145. | RVWEDRPsSA; |
| | RVWEDRPSsA; |
| | RVWEDRPssA |
| 3146. | RVWEDRPSsA |
| 3147. | RVWsPPRVHKV |
| 3148. | RVYQyIQSR |
| 3149. | RVYQyIQSRFK |
| 3150. | RVYQyIQSRFY |
| 3151. | RVYQyIQSRK |
| 3152. | RVYQyIQSRY |
| 3153. | RVYsPYNHK |
| 3154. | RVYsPYNHR |
| 3155. | RVYsPYNHY |
| 3156. | RVYSRsFSK |
| 3157. | RVYSRsFSY |
| 3158. | RVYTyIQSRF |
| 3159. | RYLGGsMDLSTF |
| 3160. | RYPsNLQLF |
| 3161. | RYPtSIASL |
| 3162. | RYQtQPVTL |
| 3163. | RYRsPEPDPYLSY |
| 3164. | SAARESHPHGVKRS |
| | AsPDDDLG |
| 3165. | SAAsPVVSSM |
| 3166. | SAEsKTIEF |
| 3167. | SAGGsAEALLSDLH |
| 3168. | SAGGsAEALLSDLHAF |
| 3169. | SAIsPKSSL |
| 3170. | SAIsPTPEI |
| 3171. | SAKsPLPSY |
| 3172. | SAMsPTHHL |
| 3173. | SAQGSDVsLTA |
| 3174. | SARGsPTRPNPPVR |
| 3175. | SAYGGLTsPGLS |
| 3176. | SAYGGLTsPGLSY |
| 3177. | SAYGGLTsPGLSYSL |
| 3178. | SDDEKMPDLE |
| 3179. | sDFHAERAAREK |
| 3180. | SDMPRAHsF |
| 3181. | SDSAQGSESHsL |
| 3182. | SDsPPRPQPAF |
| 3183. | SDsPPRPQPAFKYQ |
| 3184. | SDYAVHPMsPVGRTS |
| 3185. | SEAsPSREA |
| 3186. | SEAsPSREAI |
| 3187. | SEDsSRGAF; SEDSsRGAF; |
| | SEDssRGAF |
| 3188. | SEFKAMDsI |
| 3189. | SEFTGFSGMsF |
| 3190. | SEGsLDRLY |
| 3191. | SEGsLHRKF |
| 3192. | SEGsLHRKW |
| 3193. | SEGsLHRKY |
| 3194. | SELsPGRSV; SELsPGRSV |
| 3195. | SELtPSESL |
| 3196. | SERIMQLsL |
| 3197. | SESKsMPVL |
| 3198. | SEVsPSGVGF |
| 3199. | SEYQWITsP |
| 3200. | SFDdGSVRL |
| 3201. | SFDsGIAGL |
| 3202. | SFDsGSVRL; SFDsGsVRL; |
| | SFDSGsVRL |
| 3203. | SFLPRTLsL |
| 3204. | SGGAQsPLRYLHVL |
| 3205. | sGGDDDWTHLSSKEVDPST |
| 3206. | sGGDDDWTHLS |
| | SKEVDPSTG |
| 3207. | sGGDDDWTHLSSKEVDPST |
| | GE |
| 3208. | sGGDDDWTHLSSK |
| | EVDPSTGEL |
| 3209. | sGGDDDWTHLSSKEVDPST |
| | GELQ |
| 3210. | SGPEIFTF |
| 3211. | SGPKPLFRRMsSLVGPTQ |
| 3212. | SIDdPQKL |
| 3213. | SIDsPEKL |
| 3214. | SIDsPQKL |
| 3215. | sIELPSM |
| 3216. | SIGsPVKVGK |
| 3217. | sIISPDFSF; sIIsPDFSF; |
| | SIIsPNFSF |
| 3218. | SILsFVSGL |
| 3219. | SILsRTPSV |
| 3220. | SIMsFHIDL |
| 3221. | SIMsPEIQL |
| 3222. | SIPsGYLEL |
| 3223. | SIPtVSGQI |
| 3224. | SIRYSGHsL |
| 3225. | SISsIDREL |
| 3226. | SISsMEVNV |
| 3227. | SISStPPAV |
| 3228. | SISsVSNTF |
| 3229. | SITItPPDRYDSL |
| 3230. | SKEDKNGHDGDTHQEDDG |
| | EKsD |
| 3231. | SKSPSLSPSPPsPLEKTPL |
| 3232. | SKtVATFIL |
| 3233. | SLsSPTVTL; SLSsPTVTL; |
| | SLssPTVTL |
| 3234. | SLAsLLAKV |
| 3235. | SLAsLTEKI |
| 3236. | SLDsSNSGF; SLDSsNSGF; |
| | SLDssNSGF |
| 3237. | SLDSEDYsL |
| 3238. | SLDsLDLRV |
| 3239. | SLDsLGDVFL |
| 3240. | SLDsPGPEKM |
| 3241. | SLDsPGPEKMAL |
| 3242. | SLDsPSYVLY |
| 3243. | SLDsQQDSMKY; |
| | SLDSQQDsMKY |
| 3244. | SLEEPKQANGGAY |
| 3245. | SLEsPSYVLY |
| 3246. | SLFGGsVKL |
| 3247. | SLFKRLYsL |
| 3248. | SLFsGDEENA |
| 3249. | SLFsGSYSSL |
| 3250. | SLFsPQNTL |
| 3251. | SLFsPRRNK |
| 3252. | SLFsPRRNY |
| 3253. | SLFsSEESNL |
| 3254. | SLFSSEESNLGA |
| 3255. | SLGPIRsL |
| 3256. | SLHDIQLsL |
| 3257. | SLHsLGSVSL |
| 3258. | SLIDGyYRL |
| 3259. | SLKsPVTVK |
| 3260. | SLLAsPGHISV |
| 3261. | SLLHTSRSL |
| 3262. | SLLNKSSPVK |
| 3263. | SLLNKSsPVKK |
| 3264. | SLLNKSSPVKY |
| 3265. | SLLsELQHA |
| 3266. | SLLsLHVDL |
| 3267. | SLLsLQTEL |
| 3268. | SLLsVSHAL |
| 3269. | SLLTsPPKA |
| 3270. | SLLTsPPKV |
| 3271. | SLMsGTLESL |
| 3272. | SLMsPGRRK |
| 3273. | SLMsPGRRY |
| 3274. | SLMtISHPGL; SLMTIsHPGL; |
| | SLMtIsHPGL |
| 3275. | SLNSsPVSK |
| 3276. | SLQPRSHsV |
| 3277. | SLQsLETSV |
| 3278. | SLRRsVLMK |
| 3279. | SLRRsVLMY |
| 3280. | SLSsERYYL |
| 3281. | SLSsLLVKL |
| 3282. | SLtRSPPRV; SLTRsPPRV; |
| | SLtRsPPRV |
| 3283. | SLVDGyFRL |
| 3284. | SLYDRPAsY |
| 3285. | SLYsPVKKK |
| 3286. | SMFsPRRNK |
| 3287. | SMKsPLYLVSR |
| 3288. | SMKsPVTVK |
| 3289. | SMLNKSSPVK |
| 3290. | SMLNKSsPVKK |
| 3291. | SMLsQEIQTL |
| 3292. | SMLTsPPKA |
| 3293. | SMLTsPPKV |
| 3294. | SMMsPGRRK |
| 3295. | SMQPRSHsV |
| 3296. | SMRRsVLMK |
| 3297. | SMSsLSREV |
| 3298. | SMTRsPPRV |
| 3299. | SMYsPVKKK |
| 3300. | SNFKsPVKTIR |
| 3301. | SPSSPSVRRQL |
| 3302. | SPAASISRLsGEQVDGKG |
| 3303. | SPAsPKISF |
| 3304. | SPAsPKISL |
| 3305. | SPAsPKISM |
| 3306. | SPAsPKISV |
| 3307. | SPAsPLKEL |
| 3308. | SPDsSQSSL; SPDSsQSSL; |
| | SPDssQSSL |
| 3309. | SPDHSDHtL |
| 3310. | SPDsSQSSL |
| 3311. | sPEDEYELLMPHRISSH; |
| | SPEDEYELLMPHRIsSH; |
| | sPEDEYELLMPHRIsSH |
| 3312. | SPEKAGRRsSF |
| 3313. | SPEKAGRRsSL |
| 3314. | SPEKAGRRsSM |
| 3315. | SPEKAGRRsSV |
| 3316. | SPERPFLAILGGAKVADK |
| 3317. | SPERPFLAILGGAKVADKIQ |
| 3318. | SPFKRQLsF |
| 3319. | SPFKRQLsL |
| 3320. | SPFKRQLsM |
| 3321. | SPFKRQLsV |
| 3322. | SPFLSKRsL; SPFLsKRSL; |
| | SPFLsKRsL |
| 3323. | SPFSSRSPsL |
| 3324. | SPGLARKRsF |
| 3325. | SPGLARKRsL |
| 3326. | SPGLARKRsM |
| 3327. | SPGLARKRsV |
| 3328. | SPGsPRPAF |
| 3329. | SPGsPRPAL |
| 3330. | SPGsPRPAM |
| 3331. | SPGsPRPAV |
| 3332. | SPHsPFYQL |
| 3333. | SPHYFSPFRPY |
| 3334. | SPIAPRsPAKL |
| 3335. | SPIKVTL |
| 3336. | SPKPPTRsP |
| 3337. | SPKSGsPKSSSL |
| 3338. | SPKsPGLKA |
| 3339. | SPKsPGLKAM |
| 3340. | SPKsPGLKF |
| 3341. | SPKsPGLKL |
| 3342. | SPKsPGLKM |
| 3343. | SPKsPGLKV |
| 3344. | SPKsPTAAF |
| 3345. | SPKsPTAAL |
| 3346. | SPKsPTAAM |
| 3347. | SPKsPTAAV |
| 3348. | SPLsKIGIEL |
| 3349. | SPLsPTETF |
| 3350. | SPLTKSIsL |
| 3351. | SPPDsPGRTL |
| 3352. | sPPFPVPVYTRQAPKQVIK |
| 3353. | SPPsPARWSL |
| 3354. | SPPsPLEKTPL |
| 3355. | SPRAPVsPLKF |
| 3356. | SPRERsPAL |
| 3357. | SPRGEASsL |
| 3358. | SPRGSGsSTSL |
| 3359. | SPRLPRsPRL |
| 3360. | SPRPPNsPSI |
| 3361. | SPRPPNsPSISI |
| 3362. | SPRRsLGLAL |
| 3363. | SPRRsRSISF |
| 3364. | SPRRsRSISL |
| 3365. | SPRRsRSISM |
| 3366. | SPRRsRSISV |
| 3367. | SPRsESGGL |
| 3368. | SPRsITSTF |
| 3369. | SPRsITSTL |
| 3370. | SPRsITSTM |
| 3371. | SPRsITSTP |
| 3372. | SPRsITSTV |
| 3373. | SPRsPDRTL |
| 3374. | SPRsPGKPF |
| 3375. | SPRsPGKPL |
| 3376. | SPRsPGKPM |
| 3377. | SPRsPGKPV |
| 3378. | SPRsPGPLPGARGL |
| 3379. | SPRsPGRSF |
| 3380. | SPRsPGRSL |
| 3381. | SPRsPGRSM |
| 3382. | SPRsPGRSV |
| 3383. | SPRsPISPEL |
| 3384. | SPRsPSGLR |
| 3385. | SPRsPSTTYF |
| 3386. | SPRsPSTTYL |
| 3387. | SPRsPSTTYM |
| 3388. | SPRsPSTTYV |
| 3389. | SPRsPTPSY; SPRSPtPSY; |
| | SPRsPtPSY |
| 3390. | SPRsPVPTTL |
| 3391. | SPRssQLV |
| 3392. | SPRtPPQRF |
| 3393. | SPRtPSNTP |
| 3394. | SPRTPtPFKHAL |
| 3395. | SPRTPVsPVKF; |
| | SPRtPVSPVKF; |
| | SPRTPVSPVKF; |
| | SPRtPVSPVKF; |
| | SPRtPVsPVKF |
| 3396. | SPRtPVSPVKL; |
| | SPRTPVSPVKL; |
| | SPRtPVsPVKL |
| 3397. | SPRtPVSPVKM; |
| | SPRTPVSPVKM; |
| | SPRtPVsPVKM |
| 3398. | SPRtPVSPVKV; |
| | SPRTPVsPVKV; |
| | SPRtPVsPVKV |
| 3399. | SPsFGDPQL |
| 3400. | SPSKSPSLSPSPPsPLEKTPL |
| 3401. | SPSLSPSPPsPLEKTPL |
| 3402. | SPSsPRVRL |
| 3403. | SPSsPSVRRQF |
| 3404. | SPSsPSVRRQL |
| 3405. | SPSsPSVRRQM |
| 3406. | SPSsPSVRRQV |
| 3407. | SPSTSRSGGsSRF |
| 3408. | SPSTSRSGGsSRL |
| 3409. | SPSTSRSGGsSRM |
| 3410. | SPSTSRSGGsSRV |
| 3411. | sPTRPNPPVRNLH |
| 3412. | SPTsPFSSL |
| 3413. | SPVNKVRRVSF |
| 3414. | SPVsPMKEL |
| 3415. | SPVVHQsF |
| 3416. | SPVVHQsL |
| 3417. | SPVVHQsM |
| 3418. | SPVVHQsV |
| 3419. | SQAASSDSAQGSDVsLTA |
| 3420. | SQDsPRKL |
| 3421. | SQILRTPsL |
| 3422. | SQIsPKSWGV |
| 3423. | SRsSSVLsL; SRSsSVLsL; |
| | SRSSsVLsL; SRsSsVLsL; |
| | SRSssVLsL; SRssSVLsL; |
| | SRsssVLSL |
| 3424. | SRDKHsEY |
| 3425. | SREKHsEI |
| 3426. | SRFNRRVsV |
| 3427. | SRHsGPFFTF |
| 3428. | SRIPLVRsF |
| 3429. | SRKsFVFEL |
| 3430. | SRLSLRRSL; SRLSLRRsL; |
| | SRLsLRRsL |
| 3431. | SRLTHLsF |
| 3432. | SRLTHLsK |
| 3433. | SRLTHLsL |
| 3434. | SRLTHLSM |
| 3435. | SRLTHLsR |
| 3436. | SRLTHLsY |
| 3437. | SRMsPKAQF |
| 3438. | SRMsPKAQK |
| 3439. | SRMsPKAQL |
| 3440. | SRMSPKAQM |
| 3441. | SRMsPKAQR |
| 3442. | SRMsPKAQY |
| 3443. | SRNQsPQRL |
| 3444. | SRPsMsPTPL |
| 3445. | SRPsSSRSY; SRPSsSRSY; |
| | SRPSSsRSY; SRPssSRSY; |
| | SRPSssRSY; SRPsSsRSY; |
| | SRPsssRSY |
| 3446. | SRSSSVLsL |
| 3447. | SRTsPITRF |
| 3448. | SRTsPITRK |
| 3449. | SRTsPITRL |
| 3450. | SRTSPITRM |
| 3451. | SRTsPITRR |
| 3452. | SRTsPITRY |
| 3453. | SRWsGSHQF |
| 3454. | SRWsGSHQK |
| 3455. | SRWsGSHQR |
| 3456. | SRWsGSHQY |
| 3457. | SRYsGVNQSM |
| 3458. | SRYSRsPYSF; SRYsRSPYSF; |
| | SRYsRsPYSF |
| 3459. | SRYSRsPYSK; |
| | SRYsRSPYSK; SRYsRsPYSK |
| 3460. | SRYSRsPYSL; SRYsRSPYSL; |
| | SRYsRsPYSL |
| 3461. | SRYSRsPYSM; |
| | SRYsRSPYSM; SRYsRsPYSM |
| 3462. | SRYSRsPYSR; SRYsRSPYSR; |
| | SRYsRsPYSR |
| 3463. | SRYSRsPYSY; |
| | SRYsRSPYSY; SRYsRsPYSY |
| 3464. | SSAVDtLRS |
| 3465. | SSDIsPTRL |
| 3466. | SSDIsPTRY |
| 3467. | SSDKHsEY |
| 3468. | SSDPASQLsY; |
| | SSDPAsQLSY; SSDPAsQLsY |
| 3469. | SSDSAQGSDVsLTA |
| 3470. | SSDsETLRY |
| 3471. | SSDsPPRPQPAF |
| 3472. | SSDsPQKL |
| 3473. | SSDsPQKY |
| 3474. | SSDsPSYVLY |
| 3475. | SSDsPTNHF |
| 3476. | SSDsPTNHFF |
| 3477. | SSEIsPTRY |
| 3478. | SSEKHsEY |
| 3479. | SSEPASQLsY |
| 3480. | SSEsETLRY |
| 3481. | SSEsPQKL |
| 3482. | SSEsPQKY |
| 3483. | SSEsPSYVLY |
| 3484. | SSEsPTNHFY |
| 3485. | SSGRsPSKAVAAR |
| 3486. | SSIPSTLsL |
| 3487. | SSIsPVRL |
| 3488. | SsLPRYLGL |
| 3489. | SSMKsPLYL |
| 3490. | SSMsPLPQM |
| 3491. | SSNGKMASRRsEEKEAG |
| 3492. | SSNGKMASRRsEEKEAGEI |
| 3493. | SsPEFFM |
| 3494. | SsPIMRKKVSL |
| 3495. | sSPPFPVPVYTRQAPKQVIK |
| 3496. | SSPRsPTTTL |
| 3497. | SSSGsPHLY |
| 3498. | SSsPTHAKSAHV |
| 3499. | SSSSSGsPHLY |
| 3500. | SSsWRILGSKQSEHRP |
| 3501. | SsVPGVRLL |
| 3502. | SsVPGVRLLQ |
| 3503. | SsVPGVRLLQD |
| 3504. | SsVPGVRLLQDSVD; |
| | SSVPGVRLLQDsVD; |
| | SsVPGVRLLQDsVD |
| 3505. | SSVsPAVSK |
| 3506. | SSYPRPLtY |
| 3507. | STDIsPTRL |
| 3508. | STDIsPTRY |
| 3509. | STDKHsEY |
| 3510. | STDPASQLsY |
| 3511. | STDsETLRY |
| 3512. | STDsGLGLGcY |
| 3513. | STDsPQKY |
| 3514. | STDsPRLL |
| 3515. | STDsPSYVLY |
| 3516. | STDsPTNHFY |
| 3517. | STEIsPTRL |
| 3518. | STEIsPTRY |
| 3519. | STEKHsEY |
| 3520. | STEPASQLsY |
| 3521. | STEsETLRY |
| 3522. | STEsPQKY |
| 3523. | STEsPSYVLY |
| 3524. | STEsPTNHFY |
| 3525. | STFsTNYRSL |
| 3526. | STIAILNsV |
| 3527. | STIQNsPTKK; sTIQNSPTKK |
| 3528. | STIsLVTGETER |
| 3529. | STIsPSGAFG |
| 3530. | STIsPSGAFGLF |
| 3531. | STKsTELLL |
| 3532. | STLLAsPMLK |
| 3533. | STMsLNIITV; sTMSLNIITV; |
| | sTMsLNIITV |
| 3534. | STPsGYLEL |
| 3535. | SVsSLEVHF; SVSsLEVHF; |
| | SVssLEVHF |
| 3536. | SVAsPLTL |
| 3537. | SVDIsPTRL |
| 3538. | SVDIsPTRY |
| 3539. | SVFRHFGsFQK |
| 3540. | SVFsPSFGL |
| 3541. | SVGsDDELGPIR |
| 3542. | SVGsDYYIQL |
| | SYSFSsSSIGH; |
| | SYSFSSsSIGH; |
| | SYSFSSSsIGH; |
| | SYSFssSSIGH; SYSFsSsSIGH; |
| | SYSFSssSIGH; SYSFSsSsIGH; |
| | SYSFsssSIGH; SYSFssSsIGH; |
| | SYSFsSssIGH; SYSFSsssIGH; |
| | SYSFssssIGH |
| 3543. | sVINVFVGR |
| 3544. | SVIsDDSVL |
| 3545. | SVIsQERLSY |
| 3546. | SVIs; SVKPRRTsL; |
| | SVKsPEVQLL |
| 3547. | SVKsPVTVK |
| 3548. | SVKsPVTVY |
| 3549. | SVLPRALSL |
| 3550. | SVLsPSFQL |
| 3551. | SVLsYTSVR |
| 3552. | SVLVRQISL |
| 3553. | SVMDsPKKL |
| 3554. | SVMQSPLVGV |
| 3555. | SVPGVRLLQDsVD |
| 3556. | SVQsDQGYISR |
| 3557. | SVRRsVLMK |
| 3558. | SVRRsVLMY |
| 3559. | SVRsLSLSL |
| 3560. | SVRsPTPYK; SVRSPtPYK; |
| | SVRsPtPYK |
| 3561. | SVSRsPVPEK |
| 3562. | SVSsLEVHF |
| 3563. | SVSsSSYR |
| 3564. | SVTsPIKMK |
| 3565. | SVYsGDFGNLEV |
| 3566. | SVYsPVKKK |
| 3567. | SVYsPVKKY |
| 3568. | sYIEHIFEI |
| 3569. | SYMGHFDLL |
| 3570. | SYPsPVATSY |
| 3571. | SYPsPVPTSF |
| 3572. | sYQKVIELF |
| 3573. | SYSFsSSSIGH; |
| 3574. | SYSYSFsSSSIGH; |
| | SYSYSFSsSSIGH; |
| | SYSYSFSSsSIGH; |
| | SYSYSFSSSsIGH; |
| | SYSYSFssSSIGH; |
| | SYSYSFsSsSIGH; |
| | SYSYSFSssSIGH; |
| | SYSYSFSsSsIGH; |
| | SYSYSFsssSIGH; |
| | SYSYSFssSsIGH; |
| | SYSYSFsSssIGH; |
| | SYSYSFSsssIGH; |
| | SYSYSFssssIGH |
| 3575. | SYYsLPRSF |
| 3576. | SYYsPSIGF |
| 3577. | SYYsPSIGFSY |
| 3578. | TAIsPPLSV |
| 3579. | TAPLVPPLsPQY |
| 3580. | TASPVAVsL |
| 3581. | TATsPLTSY |
| 3582. | TDKYsKMM |
| 3583. | TEAsPESML |
| 3584. | TEDsNLRLF |
| 3585. | TELPKRLsL |
| 3586. | TEPLPEKTQEsL |
| 3587. | TESsPGSRQIQLW |
| 3588. | THKGEIRGASTPFQFRASSP |
| 3589. | THsLLLLL; tHSLLLLL; |
| | tHsLLLLL |
| 3590. | TIGEKKEPsDKSVDS |
| 3591. | TIRsPTTVL |
| 3592. | TItPPDRYDSL |
| 3593. | TKDKYMASRGQKAKsMEG |
| 3594. | TKsVKALSSLHGDD |
| 3595. | TKsVKALSSLHGDDQ |
| 3596. | TKsVKALSSLHGDDQD |
| 3597. | TLAsPSVFK |
| 3598. | TLAsPSVFKST |
| 3599. | TLAsPSVFKSV |
| 3600. | TLDsLDFARY |
| 3601. | TLEsTTVGTSV; |
| | TLEStTVGTSV; |
| | TLESTtVGTSV; |
| | TLEstTVGTSV; |
| | TLEsTtVGTSV; |
| | TLESttVGTSV; TLEsttVGTSV |
| 3602. | TLLAsPMLK |
| 3603. | TLLsPSSIKV |
| 3604. | TLMERTVsL |
| 3605. | TLSsIRHMI |
| 3606. | TLSsPPPGL |
| 3607. | TMAsPGKDNY |
| 3608. | TMAsPSVFKST |
| 3609. | TMAsPSVFKSV |
| 3610. | TMDsPGKDNY |
| 3611. | TMEsPGKDNY |
| 3612. | TMFLRETsL |
| 3613. | TMMsPSQFL |
| 3614. | TPAPSRTAsF |
| 3615. | TPAQPQRRsF |
| 3616. | TPAQPQRRsL |
| 3617. | TPAQPQRRsM |
| 3618. | TPAQPQRRsV |
| 3619. | TPAsPNREL |
| 3620. | TPASsRAQTL |
| 3621. | TPASSSSAL |
| 3622. | TPAtPTSQF |
| 3623. | TPDPSKFFSQLsSEHGGDV |
| 3624. | tPDPSKFFSQLSSEHGGDVQ |
| 3625. | TPHtPKSLL |
| 3626. | TPIsPGRASGF |
| 3627. | TPIsPGRASGM |
| 3628. | TPIsPGRASGMTTL |
| 3629. | TPIsPGRASGV |
| 3630. | TPIsPLKTGV |
| 3631. | TPIsQAQKL |
| 3632. | TPKsPGASNF |
| 3633. | TPMKKHLsL |
| 3634. | TPPPPPDtPP |
| 3635. | TPPSSEKLVSVM; |
| | TPPSSEKLVSVM; |
| | TPPssEKLVSVM |
| 3636. | TPQPSRPVsPA |
| 3637. | TPQPSRPVsPAG |
| 3638. | TPRPAsPGPSL |
| 3639. | TPRsPPLGF |
| 3640. | TPRsPPLGL |
| 3641. | TPRsPPLGLF |
| 3642. | TPRsPPLGLI |
| 3643. | TPRsPPLGLL |
| 3644. | TPRsPPLGLM |
| 3645. | TPRsPPLGLV |
| 3646. | TPRsPPLGM |
| 3647. | TPRsPPLGV |
| 3648. | TPRtPRTPQL; TPRTPRtPQL; |
| | TPRtPRtPQL |
| 3649. | TPsPARPAL |
| 3650. | TPSsFDTHF |
| 3651. | TPSsREGTL |
| 3652. | TPVsPGSTF |
| 3653. | TPVsPRLHV |
| 3654. | tPVSPTASM |
| 3655. | TPVsPVKF |
| 3656. | TPVsSANMM |
| 3657. | TRDsLLIHL |
| 3658. | TRKTPEsFL; TRKtPESFL; |
| | TRKtPEsFL |
| 3659. | TRLsPAKIVLF |
| 3660. | TRLsPAKIVLK |
| 3661. | TRLsPAKIVLR |
| 3662. | TRLsPAKIVLY |
| 3663. | TRLsPLEL |
| 3664. | TRMsTVSEL; TRMStVSEL; |
| | TRMstVSEL |
| 3665. | TRSsAVRLR |
| 3666. | TRSsPVRKL |
| 3667. | TRYPtILQL |
| 3668. | TSAsPGKDNY |
| 3669. | TSDsPGKDNY |
| 3670. | TSDsPPHNDI |
| 3671. | TSDtPDYLLKY |
| 3672. | TSEsPGKDNY |
| 3673. | TSEtPDYLLKY |
| 3675. | TSFSVGsDDELGPIR |
| 3676. | TSGPGSRISSSsF |
| 3677. | TSIsPALAR |
| 3678. | TSIsPSRHGAL |
| 3679. | TSPsYIDKL |
| 3680. | TSVsPAPDK |
| 3681. | TTAsPGKDNY |
| 3682. | TTASPGKDNY |
| 3683. | TTDPLIRWDsY |
| 3684. | TTDsPGKDNY |
| 3685. | TTDtPDYLLKY |
| 3686. | TTEsPGKDNY |
| 3687. | TTEtPDYLLKY |
| 3688. | TTKsVKALSSLHG |
| 3689. | TTKsVKALSSLHGDD |
| 3690. | TTKsVKALSSLHGDDQ |
| 3691. | TTKsVKALSSLHGDDQD |
| 3692. | TTKsVKALSSLHGDDQDS |
| 3693. | TTKSVKALSSLHGDDQDsE |
| | D |
| 3694. | TTKSVKALSSLHGDDQDsE |
| | DE |
| 3695. | TVDsPPWQL |
| 3696. | TVFsPTLPAA |
| 3697. | TVFsPTLPAAR |
| 3698. | TVKQKYLsF |
| 3699. | TVMsNSSVIHL |
| 3700. | TVNsPAIYK |
| 3701. | TVNsPAIYKF |
| 3702. | TVtPVPPPQ |
| 3703. | TVYSSEEAELLK; |
| | TVYSsEEAELLK; |
| | TVYssEEAELLK |
| 3674. | TsFADEL |
| 3704. | TYEGIFKtL |
| 3705. | VADSPAEVAL |
| 3706. | VADSPRDTASL |
| 3707. | VADtSIQKL |
| 3708. | VAKRLSL |
| 3709. | VAMPVKKSPRRSSSDEQGLS |
| | YSSLKNV |
| 3710. | VEFPHsPEI |
| 3711. | VEKLPDsPAL |
| 3712. | VELsPAR |
| 3713. | VELsPARSW |
| 3714. | VETsFRKLSF; VETSFRKLsF; |
| | VETsFRKLsF |
| 3715. | VGsDDELGPIR |
| 3716. | VIDsQELSKV |
| 3717. | VIMsIRTKL |
| 3718. | VIsDGGDSEQF |
| 3719. | VLAsPLKTGR |
| 3720. | VLDsPASKK |
| 3721. | VLEKsPGKLLV |
| 3722. | VLFPEsPARA |
| 3723. | VLFRtPLASV |
| 3724. | VLFSsPPQM; VLFsSPPQM; |
| | VLFssPPQM |
| 3725. | VLIENVAsL |
| 3726. | VLIGsPKKV |
| 3727. | VLIGsPKKY |
| 3728. | VLKGsRSSEL |
| 3729. | VLKGsRSSEV |
| 3730. | VLKSRKssVTEE |
| 3731. | VLKVMIGSPK |
| 3732. | VLKVMIGSPKK |
| 3733. | VLKVMIGSPKKK |
| 3734. | VLLsPVPEL |
| 3735. | VLLsPVPEV |
| 3736. | VLMKsPSPAL; |
| | VLMKSPsPAL; |
| | VLMKsPsPAL |
| 3737. | VLMKsPsPAV |
| 3738. | VLQTPPYVK |
| 3739. | VLQtPPYVKK |
| 3740. | VLQtPPYVKY |
| 3741. | VLSDVIPsI |
| 3742. | VLSSLtPAKV |
| 3743. | VLTsNVQTI |
| 3744. | VLYsPQMAL |
| 3745. | VMDsPVHL |
| 3746. | VMFPGNsPSY |
| 3747. | VMFRtPLASV |
| 3748. | VMIGsPKKV |
| 3749. | VMIGsPKKY |
| 3750. | VMKVMIGSPK |
| 3751. | VMKVMIGSPKK |
| 3752. | VMKVMIGSPKKK |
| 3753. | VMKVMIGSPKKY |
| 3754. | VMLsPVPEL |
| 3755. | VMLsPVPEV |
| 3756. | VMQsPLVGV |
| 3757. | VMQTPPYVK |
| 3758. | VMQtPPYVKK |
| 3759. | VMTsLQQEY |
| 3760. | VPAsSTSTL |
| 3761. | VPAtHGQVTY |
| 3762. | VPGVRLLQDsVD |
| 3763. | VPHHGFEDWsQIR |
| 3764. | VPKKPPPsP |
| 3765. | VPKSGRsSSL; VPKSGRSsSL; |
| | VPKSGRSSsL; VPKSGRsSsL; |
| | VPKSGRSssL; VPKSGRsSsL |
| 3766. | VPKsPAFAL |
| 3767. | VPLIRKKsL |
| 3768. | VPREVLRLsF |
| 3769. | VPREVLRLsL |
| 3770. | VPREVLRLsM |
| 3771. | VPREVLRLsV |
| 3772. | VPRPERRSsL |
| 3773. | VPRPERRssL |
| 3774. | VPRPERRsSL |
| 3775. | VPRsPKHAHSSSF |
| 3776. | VPRsPKHAHSSSL |
| 3777. | VPRsPKHAHSSSM |
| 3778. | VPRsPKHAHSSSV |
| 3779. | VPRsPVIKI |
| 3780. | VPRtPSRERSSSA |
| 3781. | VPRtPVGKF |
| 3782. | VPSsPLRKA |
| 3783. | VPTsPKGRLL |
| 3784. | VPTsPKSSL |
| 3785. | VPtTSSSL; VPTtSSSL; |
| | VPTTsSSL; VPttSSSL; |
| | VPtTsSSL; VPTtsSSL; |
| | VPttsSSL |
| 3786. | VPVsGTQGL |
| 3787. | VPVsNQSSL |
| 3788. | VPVsPGQQL |
| 3789. | VPVsSASEL |
| 3790. | VPVsVGPSL |
| 3791. | VRAsKDLAQ |
| 3792. | VRLLQDsVD |
| | VYLPTHTsLLNLT; |
| | VYLPTHtsLLNLT |
| 3793. | VRQsPGPAL |
| 3794. | VRQsVTSFPDADAFHHQ |
| 3795. | VRTPSVQsL |
| 3796. | VRYsQLLGL |
| 3797. | VSDsPSHIA |
| 3798. | VSDsPSHIAT |
| 3799. | VSKVMIGsPKKV |
| 3800. | VSKVMIGsPKKY |
| 380I. | VsPFQEL |
| 3802. | VSPSKSPSLSPSPPsPLEKTPL |
| 3803. | VSsPPPYTAY |
| 3804. | VSSSDsPPRPQPAF |
| 3805. | VSSsPRELL |
| 3806. | VTKsSPRAL; VTKSsPRAL; |
| | VTKssPRAL |
| 3807. | VTQtPPYVKK |
| 3808. | VTtPNRLIY |
| 3809. | VTtPTGYKY |
| 3810. | VTTSTRTYsLG |
| 3811. | VVDsPGQEVL |
| 3812. | VVsEVDIAKAD |
| 3813. | VVSsPKLAPK |
| 3814. | VYIPMsPGAHHF |
| 3815. | VYLPTHTsL |
| 3816. | VYLPTHtSLL; |
| | VYLPTHTSLL; VYLPTHtsLL |
| 3817. | VYLPTHtSLLN; |
| | VYLPTHTsLLN; |
| | VYLPTHtsLLN |
| 3818. | VYLPTHtSLLNL; |
| | VYLPTHTsLLNL; |
| | VYLPTHtsLLNL |
| 3819. | VYLPTHtSLLNLT; |
| | YLDSGIHsGA |
| 3820. | VYTyIQSRF |
| 3821. | WEFGKRDsL |
| 3822. | WIGLNSLsF |
| 3823. | WTHLsSKEVDPS |
| 3824. | WTHLsSKEVDPSTG |
| 3825. | YAFEGTGsL |
| 3826. | YARsVHEEF |
| 3827. | YASSKLLKI; YASsKLLKI; |
| | YAssKLLKI |
| 3828. | YAVPRRGsL |
| 3829. | YAYDGKDyI |
| 3830. | YCIsPSTAAQF |
| 3831. | YEFsPVKML |
| 3832. | YEGsPIKV |
| 3833. | YEGsPIKVT |
| 3834. | YEGsPIKVTL |
| 3835. | YEGsPIKVTL |
| 3836. | YEKLsAEQSPPP |
| 3837. | YEsPGKIFL |
| 3838. | YFsPFRPY |
| 3839. | YGDRTStF |
| 3840. | YGITsPISL |
| 3841. | YHLsPRAFLHY |
| 3842. | YIKtELISV |
| 3843. | yIQSRF |
| 3844. | YLAsLEKKL |
| 3845. | YLDsGIHSG |
| 3846. | YLDsGIHSGA; |
| | YLDSGIHsGA; |
| | YLDsGIHsGA; |
| | YLDsGIHSGV; |
| 3847. | YLDsGIHsGV |
| 3848. | yLGLDVPV |
| 3849. | YLGsISTLVTL |
| 3850. | YLIHsPMSL |
| 3851. | YLLSPLNTL |
| 3852. | YLLsPTKLPSI |
| 3853. | YLLsPTKLPSV |
| 3854. | YLPsFFTKL |
| 3855. | YLPTHTsLL |
| 3856. | yLQSRYYRA |
| 3857. | YLQsRYYRA |
| 3858. | yLQsRYYRA |
| 3859. | YLRsVGDGETV |
| 3860. | YLSDsDTEAKL |
| 3861. | YLVsPITGEKI |
| 3862. | YMDsGIHSGA |
| 3863. | YMDsGIHSGV |
| 3864. | YPDPHsPFAV |
| 3865. | YPGGRRsSL |
| 3866. | YPHsPGSQY |
| 3867. | YPLQIsPVSSY |
| 3868. | YPLsPAKVNQY |
| 3869. | YPLsPTKISEY |
| 3870. | YPLsPTKISQY |
| 3871. | YPRLSIPNL |
| 3872. | YPRsFDEVEGF |
| 3873. | YPRsFDEVEGM |
| 3874. | YPRsFDEVEGV |
| 3875. | YPRsFDEVEGVF |
| 3876. | YPRsFDEVEGVL |
| 3877. | YPRsFDEVEGVM |
| 3878. | YPRsFDEVEGVV |
| 3879. | YPSFRRsSL; YPSFRRSsL; |
| | YPSFRRssL |
| 3880. | YPSsPRKAL |
| 3881. | YPSsPRKF |
| 3882. | YPSsPRKL |
| 3883. | YPSsPRKM |
| 3884. | YPSsPRKV |
| 3885. | YPVsPKQKY |
| 3886. | YPYEFsPVKM |
| 3887. | YQLsPTKLPSI |
| 3888. | YQLsPTKLPSV |
| 3889. | YQRPFsPSAY |
| 3890. | YQRsFDEVEGF |
| 3891. | YQRsFDEVEGL |
| 3892. | YQRsFDEVEGM |
| 3893. | YQRsFDEVEGV |
| 3894. | YQRsFDEVEGVF |
| 3895. | YQRsFDEVEGVL |
| 3896. | YQRsFDEVEGVM |
| 3897. | YQRsFDEVEGVV |
| 3898. | YRNDSSSsL |
| 3899. | YRRsVPTWL |
| 3900. | YRYsPQSFL |
| 3901. | YSDRsSGGSY |
| 3902. | YSEsRSSLDY |
| 3903. | YsFcGTVEY |
| 3904. | YSFsPSKSY |
| 3905. | YSFSSSsIGH |
| 3906. | YSLDsPGPEK |
| 3907. | YSLDsPGPEKM |
| 3908. | YSLDsPGPEKMAL |
| 3909. | YSLsPRPSY |
| 3910. | YSLsPSKSY |
| 3911. | YSLsPSKSYKY |
| 3912. | YSsLVRVL |
| 3913. | YSTtPGGTLY |
| 3914. | YTAGtPYKV |
| 3915. | YTDSESSAsL |
| 3916. | YTsSRDAFGY; |
| | YTSsRDAFGY; |
| | YTssRDAFGY |
| 3917. | YVDAETsL |
| 3918. | YVKLTPVsL |
| 3919. | YVPDsPALL |
| 3920. | YVSsPDPQL |
| 3921. | YYTAGSSSPTHAKSAHV |
| 3975. | RRLsFSTRL |
| 3976. | RRRsRVFDL |
| 3977. | RSFsPKSPLEL |
| 3978. | RSHsLHYLF |
| 3979. | RSKsSImYF |
| 3980. | RSRsDNALHL |
| 3981. | RSVsPTTEM |
| 3982. | RSYsRLETL |
| 3983. | RTLHsPPLQL |
| 3984. | RVAsPKLVm |
| 3985. | SISVQVNSIKFDsE |
| 3986. | SPFQSSPLsL |
| 3987. | SPGsPLHSL |
| 3988. | SPGsPLVSm |
| 3989. | SPHtPSTHF |
| 3990. | SPPNLtPKPL |
| 3991. | SPRDsPAVSL |
| 3992. | sPRsPGRSL |
| 3993. | SPRsPQLSDF |
| 3994. | STsSGRLLY |
| 3995. | SVKsPEVQLL |
| 3996. | TKSsPLKI |
| 3997. | VLVVDTPsI |
| 3998. | VPRPStPSRL |
| 3999. | yAQPQTTTPLPAVSG |
| 4000. | yYPDPHsPFAV |

In the listing above, the number preceding each sequence or group of sequences corresponds to the SEQ ID NO: in the Sequence Listing submitted herewith. Also, lowercase “s” refers to a modified (e.g., phosphorylated) serine, lowercase “t” refers to a modified (e.g., phosphorylated) threonine, lowercase “y” refers to a modified (e.g., phosphorylated) tyrosine, lowercase “n” refers to a modified (e.g., glycosylated, in some embodiments with hexose-GlcNAc) asparagine, lowercase “k” refers to an N-terminal modified lysine, and lowercase “c” refers to a modified (e.g., cysteinylated or methyl esterified (e.g., homocysteine) cysteine. Lowercase “w” refers to a modification of a tryptophan to kynurenine. In some embodiments, the sequences APPsTSAAAL (SEQ ID NO: 116), IPVsKPLSL (SEQ ID NO: 705), IPVsSHNSL (SEQ ID NO: 708), KPPTsQSSVL (SEQ ID NO: 1033), KPPVsFFSL (SEQ ID NO:1034), KPTLYnVSL (SEQ ID NO: 1079), PPStSAAAL (SEQ ID NO: 1487), PPSTsAAAL (SEQ ID NO: 1487), and RPPQsSSVSL (SEQ ID NO: 2126) can be modified with 2-hexose-GlcNAc, hexose-di-GlcNAc, and/or hexose-GlcNAc. (AcS) refers to an acylated serine.

With respect to the modifications of the sequences shown above, the particular phosphorylation sites noted in lowercase are exemplary only, and it is understood that any or all serines, threonines, and/or tyrosines that are identified in upper case letters can also be modified (e.g., phosphorylated).

In some embodiments, a peptide of the presently disclosed subject matter is one that is set forth in Table 7:

1. A composition comprising, consisting essentially of, or consisting of at least or about 1, 2, 3, 4, 5, 6, 7, 8, 9, 10, or more synthetic peptides, wherein each synthetic peptide:
(i) is between 8 and 50 amino acids long; and
(ii) comprises, consists essentially of, or consists of an amino acid sequence selected from the group consisting of SEQ ID NOs: 1-3921 and 3975-4000,
and further wherein said composition optionally has the ability to stimulate a T cell-mediated immune response to at least one of the synthetic peptides and/or is capable of eliciting a memory T cell response to at least one of the synthetic peptides.

2. The composition of claim 1, wherein at least one of the synthetic peptides comprises a substitution of a serine residue with a homo-serine residue.

3. The composition of claim 1, wherein at least one of the synthetic peptides is a phosphopeptide comprising phosphoserine, phosphothreonine, or phosphotyrosine.

4. The composition of claim 1, wherein at least one of the synthetic peptides comprises, consists essentially of, or consists of a phosphopeptide set forth in Table 6.

5. The composition of claim 1, wherein at least one of the synthetic peptides comprises a phosphopeptide mimetic comprising a mimetic of phosphoserine, phosphothreonine, or phosphotyrosine.

6. The composition of claim 5, wherein at least one of the synthetic peptides comprises a phosphopeptide mimetic of a phosphopeptide set forth in Table 6

7. The composition of claim 6, wherein the phosphopeptide mimetic is resistant to dephosphorylation by a phosphatase enzyme.

8. The composition of claim 6, wherein the phosphopeptide mimetic is a synthetic molecule in which a phosphorous atom is linked to a serine, threonine, or tyrosine amino acid residue through a carbon.

9. The composition of claim 1, wherein the composition is immunologically suitable for use in a subject who has or is at risk of developing a cancer and/or a tumor, wherein the cancer and/or the tumor is optionally a breast cancer and/or a tumor, a colorectal cancer and/or a tumor, an esophageal cancer and/or a tumor, an intrahepatic cholangiocarcinoma (bile duct) cancer and/or a tumor, a leukemia, a lymphoma, a melanoma, a head and neck cancer and/or a tumor, ovarian cancer and/or a tumor, pancreatic cancer and/or a tumor, a cancer and/or a tumor of a tonsil, a lung cancer and/or a tumor, a cervical cancer and/or a tumor, a cancer and/or a tumor of partially transformed T-cells, a placental cancer and/or a tumor, a liver cancer and/or a tumor, optionally hepatocellular carcinoma (HCC), and/or a kidney cancer and/or a tumor.

10. The composition of claim 1, wherein the composition comprises, consists essentially of, or consists of at least 2, 3, 4, or 5 different peptides.

11. The composition of claim 1, wherein the composition comprises, consists essentially of, or consists of at least 10 different peptides.

12. The composition of claim 1, wherein the composition comprises, consists essentially of, or consists of at least 15 different peptides.

13. The composition of claim 1, wherein at least one of the synthetic peptides is capable of binding to an MHC class I molecule selected from the group consisting of an HLA-A*0201 molecule, an HLA A*0101 molecule, an HLA A*0301 molecule, an HLA B*4402 molecule, an HLA B*0702 molecule, an HLA B*2705 molecule, an HLA *A1101 molecule, an HLA *A2301 molecule, an HLA *A2402 molecule, an HLA *B0801 molecule, an HLA *B1401 molecule, an HLA *B1402 molecule, an HLA *B1501 molecule, an HLA *B1503 molecule, an HLA *B1510 molecule, an HLA *B1511 molecule, an HLA *B1518 molecule, an HLA *B4001 molecule, an HLA *B4901 molecule, an HLA *C0303 molecule, an HLA *C0304 molecule, an HLA *C0501 molecule, an HLA *0602 molecule, an HLA *0701 molecule, an HLA *0702 molecule, and an HLA *0704 molecule.

14. The composition of claim 1, wherein the composition is capable of increasing the 5-year survival rate of a subject treated with the composition by at least 20 percent relative to average 5-year survival rates that could have been expected without treatment with the composition.

15-17. (canceled)

18. The composition of claim 1, further comprising at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

19. The composition of claim 1, wherein the composition further comprises an adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof.

20-29. (canceled)

30. A method for treating and/or preventing cancer comprising administering to a subject in need thereof a therapeutically effective dose of a composition of claim 1 and/or a composition comprising, consisting essentially of, or consisting of at least one peptide comprising an amino acid sequence as set forth in Tables 3-6.

31. The method of claim 30, wherein the cancer is selected from the group consisting of breast cancer, colorectal cancer, esophageal cancer, intrahepatic cholangiocarcinoma (bile duct) cancer, leukemia, lymphoma, melanoma, head and neck cancer, ovarian cancer, pancreatic cancer, a cancer of a tonsil, lung cancer, cervical cancer, cancer of partially transformed T-cells, placental cancer, liver cancer, hepatocellular carcinoma (HCC), and kidney cancer, and the at least one peptide comprises, consists essentially of, or consists of an amino acid sequence as set forth in Tables 3-6.

32. (canceled)

33. A method for treating and/or preventing hepatocellular carcinoma (HCC) and/or esophageal cancer comprising administering to a subject in need thereof a therapeutically effective dose of a composition of claim 1 or a composition comprising, consisting essentially of, or consisting of at least one peptide that peptide comprises, consists essentially of, or consists of an amino acid sequence as set forth in Tables 3-6 in combination with a pharmaceutically acceptable carrier.

34-42. (canceled)

43. A kit comprising at least one peptide composition comprising at least one peptide comprising, consisting essentially of, or consisting of an amino acid sequence as set forth in any of Tables 3-6, and a cytokine and/or an adjuvant.

44-96. (canceled)
